(19) United States
(12) Patent Application Publication
(10) Pub. No.: US 2013/0287790 A1
(43) Pub. Date: Oct. 31, 2013

(54) USE OF JAM-A IN DIAGNOSING AND TREATING LEUKEMIA

(71) Applicant: The Board of Trustees of the Leland Standford Junior University, Palo Alto, CA (US)

(72) Inventors: Faye Yuan-yi Hsu, Palo Alto, CA (US); Garry Nolan, San Francisco, CA (US); Yury Goltsev, Stanford, CA (US)

(21) Appl. No.: 13/829,229

(22) Filed: Mar. 14, 2013

Related U.S. Application Data
(60) Provisional application No. 61/618,554, filed on Mar. 30, 2012.

(51) Int. Cl. C12Q 1/68

(52) U.S. Cl. CPC C12Q 1/6886
USPC 424/153.1; 435/6.11; 435/7.23; 435/29; 506/9; 424/178.1; 424/649

(57) ABSTRACT

Methods, compositions, and kits are provided for the use of JAM-A in diagnosing and treating leukemia. These methods, compositions, and kits find many uses, for example in diagnosing an individual with a leukemia, classifying a leukemia, providing a prognosis to an individual with a leukemia, treating an individual with a leukemia, screening candidate agents for the ability to treat a leukemia, and in basic research to better understand the molecular and cellular basis of leukemia.

### CROSS REFERENCE TO RELATED APPLICATIONS

Pursuant to 35 U.S.C. §119 (e), this application claims priority to the filing date of the U.S. Provisional Patent Application Ser. No. 61/618,554 filed Mar. 30, 2012; the disclosure of which are herein incorporated by reference.

### GOVERNMENT RIGHTS

This invention was made with government support under CA034233 awarded by the National Institutes of Health. The Government has certain rights in the invention.

### FIELD OF THE INVENTION

This invention pertains to diagnosing and treating leukemia.

### BACKGROUND OF THE INVENTION

Risk classification and outcome prediction for patients with leukemia have to date been limited to observations of cytogenetic aberrations and gene-specific mutations. However, the current classification system does not fully reflect the molecular heterogeneity of the disease. Thus, there is a need in the art for more tools for diagnosing leukemia, classifying leukemia, providing a prognosis for patients with leukemia, and determining the most appropriate therapy for patients with leukemia. The development of such tools will also contribute to our understanding of the underlying causes of such malignancies and the development of novel treatments. The present invention addresses these issues.

### SUMMARY OF THE INVENTION

Methods, compositions, and kits are provided for the use of JAM-A and JAM-A based agents in diagnosing and treating leukemia. These methods, compositions, and kits find many uses, for example in diagnosing an individual with a leukemia, classifying a leukemia, providing a prognosis to an individual with a leukemia, treating an individual with a leukemia, screening candidate agents for the ability to treat a leukemia, and basic research to better understand the molecular and cellular basis of leukemia.

In some aspects of the invention, methods are provided for providing a diagnosis or a prognosis for an individual with leukemia, the methods comprising measuring JAM-A in a hematologic sample, e.g. a peripheral blood mononuclear cell (PBMC) sample, from an individual, and providing a diagnosis or prognosis based on the measurement. In some embodiments, the measuring step comprises contacting the sample with a JAM-A probe, and detecting the amount of JAM-A in the sample. In other embodiments, the measuring step comprises contacting the sample with a JAM-A probe, and detecting the number of JAM-A+ cells in the sample. In some embodiments, the JAM-A probe is an antibody. In other embodiments, the JAM-A probe is a nucleic acid. In some embodiments, the method further comprises comparing the measurement to the measurement of JAM-A in a hematologic sample from a control. In some such embodiments, the control is a sample from a healthy individual. In some such embodiments, the control is a sample from an individual with leukemia. In some embodiments, the individual has AML. In certain embodiments, the method further comprises genotyping the patient for a NPM1 AML mutation.

In some aspects of the invention, a method is provided for classifying a leukemia as a Type 1 or Type 2 AML. In some embodiments, the method comprises measuring the number of JAM-A+ cells in a hematologic sample, e.g. a PBMC, from a patient with a leukemia, and classifying the leukemia as a Type 1 AML or a Type 2 AML based on the number of JAM-A+ cells. In some embodiments, the method further comprises comparing the number of JAM-A+ cells to the number of JAM-A+ cells in a control sample, e.g. a hematologic sample from a Type 1 AML patient, a hematologic sample from a Type 2 AML patient. In other embodiments, the method for classifying a leukemia as a Type 1 or Type 2 AML comprises stimulating, with G-CSF, a population of blast cells a hematologic sample from an individual with leukemia; measuring the number of G-CSF-responsive cells (GRCs) and/or the number of G-CSF non-responsive cells (NGRCs) in the stimulated sample; and classifying the leukemia as a Type 1 AML or a Type 2 AML based on the number of GRCs and/or NGRCs in the stimulated sample. In some embodiments, the methods further comprises providing a prognosis based upon the classification, wherein a classification of Type1 AML indicates better overall survival for the AML patient and a classification of Type 2 AML indicates worse overall survival for the AML patient.

In some aspects of the invention, methods are provided for depleting G-CSF-responsive cells (GRCs) in an individual with leukemia, the method comprising contacting the G-CSF-responsive cells (GRCs) with a JAM-A+-binding agent in an amount effective to deplete the GRCs. In some embodiments the depletion of GRCs treats the leukemia. In some embodiments, the leukemia is AML. In some embodiments, the contacting occurs ex vivo, and the method further comprises returning the non-GRCs to the individual. In certain such embodiments, the JAM-A binding agent is conjugated to a therapeutic moiety that promotes cell death in GRCs, e.g. a cytotoxin. In other such embodiments, the JAM-A binding agent is an affinity reagent that is used to remove the contacted GRCs, e.g. by fluorescence activated cell sorting (FACS), magnetic bead cell sorting (MACS), or immunopanning, and returning the enriched population of cells to the individual. In other embodiments, the contacting occurs in vivo, wherein the JAM-A binding agent is conjugated to a therapeutic moiety that promotes cell death in GRCs, e.g. a cytotoxin, a polypeptide that targets the cell for ADCC or CDC-dependent death. In other such embodiments, the therapeutic moiety is a small molecule or polypeptide that alters the activity of the cell.

In some aspects of the invention, a method is provided for modulating the activity of G-CSF responsive cells (GRCs) in an individual, the method comprising administering to the individual an effective amount of an agent, e.g. a polypeptide, a small molecule, or an siRNA, that modulates the activity of the JAM-A signaling pathway. In some embodiments, the method finds use in treating an individual for leukemia. In some embodiments, the leukemia is AML.

In some embodiments, a method for screening a candidate agent for the ability to treat leukemia is provided, the method comprising comparing the viability, proliferation rate, or metastatic potential of JAM-A+ cells contacted with agent with the viability, proliferation rate, or metastatic potential of JAM-A+ cells not contacted with agent, wherein a decrease in viability, proliferation rate, or metastatic potential in the JAM-A+ cells contacted with agent indicates that the agent is able to treat leukemia. In some embodiments, the cells are also contacted with a JAM-A modulator. In some embodiments, the cells are also contacted with one or more cytokines that activate JAM-A+ cells. In some embodiments, the leukemia is AML.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1. Inhibiting RNases after fixation allows preservation of RNA in fixed and permeabilized cells. (A) U937 cell line RNA purified from fresh (Control) or fixed and permeabilized cells treated according to the protocol described in the Materials and Methods section were analyzed on an 1.5% agarose gel (lanes 1-4). (B, C) Live T cells (CD3+) and monocytes (CD33+) were sorted from PBMCs of normal donor 1 (ND1) and ND2. Populations from ND3 PBMCs were sorted from cells stained after fixing and permeabilization. RNA was prepared from all sorted populations and gene expression values were determined by microarray screening. (B) Correspondence between the gene expression in fixed and permeabilized (x-axis) versus live (y-axis) human T cells measured by microarray screening. (C) Heatmaps corresponding to the difference between log 2 gene expression values observed in T cells and monocytes for the top 1500 genes differentially expressed between T cells and monocytes from ND1 and ND2 (RNA from live cells) and from fixed and permeabilized cells of the ND3. Genes were sorted according to Log 2 folds observed in live cells of ND1. Variability between the heatmaps for fresh and fixed cells (e.g. ND1 and ND3) does not exceed the variability produced by difference between ND individuals (ND1 vs ND2).

FIG. 2. Gene expression in G-CSF signaling subsets points to existence of two types of AML patients. (A) Experiment design. Frozen PBMCs were thawed rested in culture medium for 1 h. Following a 15-min stimulation with cytokines, cells were fixed, permeabilized, post-fixed, and stained with a cocktail of antibodies to surface markers and intracellular proteins. Stained cells were sorted according to signaling response. RNA was purified from sorted populations and analyzed by microarray screening. (B) Gene expression profiling of sorted populations was performed by U133 Plus2 microarrays (Affymetrix). Expression data (1500 genes, selected based on highest standard deviation across the dataset) from sorted AML cell subsets (G-CSF-responsive and non-responsive cells) was co-clustered with a collection of gene expression data from T cells, mature CD33+ monocytes, and progenitor cells (CD34+CD38+) from ND1, ND2 and ND3. For Type 1 patients hierarchical clustering revealed co-segregation of cells positive for phosphorylated Stat5 (marked with red +) with progenitors and of cell negative for Stat5 (marked with red −) with mature cells type. In Type 2 patient samples both signaling subsets (marked with blue + and −) co-clustered with primitive cell types. (C-D) PCA and PAM silhouette profile width analysis support existence of two major data types corresponding to cells not responding to G-CSF in Type1 patients and all the rest subsets in AML subset microarray data. (E) Expression of 700 genes differentially expressed in the cell subsets of Type 1 AML patients and levels of expression of the same genes in various blood cell subsets taken from a previously published study (Novershtern, N., et al. (2011). Densely interconnected transcriptional circuits control cell states in human hematopoiesis. Cell 144, 296-309).

FIG. 3 Signaling threshold and distance between the GRC and NGRC separates Type1 and Type2 patients. (A, B) Levels of phosphorylation of Stat5 and Stat3 in response to activation with G-CSF in peripheral blood of 11 AML patients were measured. For microarray screening, cells displaying activated Stat5 (upper two quadrants marked with +, red in Type 1 patients and blue in Type 2 patients) were sorted separately from cells with little phosphorylated Stat5 (lower left quadrant marked with −). (C) Cytometric analysis of live cells of Type 1 and Type 2 patients using CD11a and CD321 staining. Note distinct pattern observed in Type 1 and Type 2 blasts. Red gate marks CD321^{high}CD11^{low} cells prevalent in Type 2 patients. (D) Clustering of patients in two-dimensional space defined by Euclidean distance between the subsets and the percentage of cells responding to G-CSF in the PBMCs of the patient. Euclidean distance between the subsets was calculated using the data from 500 genes selected based on highest differential expression between the mature myeloid cells versus HSC and CMP in (Broad Inst., MIT (Novershtern et al., supra)). Red line indicates 13.5% signaling threshold separating Type1 form Type2 patients.

FIG. 4. Deconvolution analysis reveals differentiation status of AML cell subsets responding to various cytokines. (A, B) Expression values (asinh(natural value)) of top 700 genes selected according to the level of standard deviation in both current and 2004 cohort microarray data predicted by deconvolution analysis performed on unsorted gene expression data from Flt3-positive patients analyzed in 2004 study as compared to gene expression values measured experimentally in cells that (A) did or (B) did not respond to G-CSF in the current study. (C) Top panel—Average gene expression profiles of cells responding or not responding to G-CSF, GMCSF, IL6, IFNγ, II3 were predicted by de-convolution analysis. Computationally derived gene expression data (asinh(natural value)) was hierarchically clustered and top 700 genes differentially expressed between these profiles were visualized on the heatmap. Bottom panel shows how genes differentially expressed between the predicted cytokine response subsets are expressed at various stages of hematopoietic differentiation. Bottom panel—Expression levels of the 700 genes used for the top panel in indicated blood cell subsets (Novershtern et al., supra). Note similarity of G-CSF and IFN responding cells to primitive cells and of IL6 responding cells to mature cells. GM-CSF and IL3 responders occupy intermediate positions between the mature and immature cells. (D) Cancer clone maturation diagram representing relative positions of cells responding to cytokines.

FIG. 5. GRCs of Type1 AML patients are enriched within CD321 positive subset. CD11a, CD36^{high} cells do not respond to G-CSF potentiation. (A) Microarray data showing CD321 expression levels (normalized to minimal expression value) in signaling subsets from Type1 and Type2 patients. Data corresponding to individual patients is shaded in alternation. Blue bars correspond to gene expression in NGRCs, red − in GRCs. Note that CD321 is differentially expressed in the subsets of Type1 patients and equally highly expressed in the subsets of Type2 patients. (B, C, D) 2D flow plots of p-STAT5 versus surface markers (CD321, CD36, CD11a) observed in blasts of NL101 treated with G-CSF. (E) Gates for sorting the CD321^{high}CD36^{low} and CD36^{high}CD321^{low} populations from Type1 patients for colony assays as observed in blasts of NL101. (F, G) Distribution of Stat5 with respect to CD321 and CD34 in normal human bone marrow treated by G-CSF and in PBMC of selected Type1 and Type2 patients. (H) Clustering of Type1 NL102 PBMC cells by “Spade” algorithm (Qiu et al., 2011). The minimal spanning tree was constructed based on CD321 and CD34. The trees are colored according to CD34, CD321 and phospho-Stat5 levels. Note localization of phospho-Stat5 high cells within the cluster with high CD321 and not within the cluster with high CD34.

FIG. 6. Interaction of signaling subsets in Type 1 samples—NGRCs activate GRCs. (A) Live cells from NL101 and NL102 Type1 patients were sorted according to the pattern of CD321 versus CD36 expression (see FIG. 5E). After sorting, cells were incubated for 24 hours in RPMI with 10% FCS. The cytokine expression profile was then examined using a Luminex assay. Cytokines with 10-fold or more difference in expression between CD321^{high} and CD321^{low} populations from both patients are shown in the table. (B) Total blasts from NL101 patients were activated either by conditioned medium from sorted subsets (middle and right column) or by plain medium (left column). There was a pronounced response of CD321^{high} cells to conditioned medium from CD321^{low}CD36^{high} cells − red arrow. (C) Total blasts from NL101 were activated by indicated cytokines. GM-CSF elicited responses in CD321^{high} cells similar to that observed with conditioned medium. Luminex data shows presence of GM-CSF in conditioned medium from CD36+ cells of both NL101 and NL102 patients. Red arrows point to response to GM-CSF and IL-3 observed in CD321^{high} cells.

FIG. 7. Prognostic significance of CD321 gene expression (A), (D), (G) CD321 expression levels in unsorted blasts reveal two subgroups of patients. Histogram and probability density of CD321 expression levels (RMA value for 223000_s_at probeset) measured in a public datasets composed of samples with Normal Karyotype (Metzeler, K. H., et al. (2008). An 86-probe-set gene-expression signature predicts survival in cytogenetically normal acute myeloid leukemia. Blood 112, 4193-4201). A threshold score of 1887 (corresponding to local minimum on a probability density curve marked with dashed red line) was chosen to distinguish two groups of patients. (B) AML patients of (Metzeler et al., supra) dataset with overall high CD321 expression have less chance of survival. Survival analysis (Kaplan-Meier estimator) of CD321^{high} and CD321^{low} patients suggests that with a high probability CD321^{high} patients have low chance of survival. (C) Incorporating NPM1 status increases the robustness of CD321 based survival prediction. Survival analysis (Kaplan-Meier estimator) done only on NPM1-positive patients shows more robust survival stratification. (D), (G) Histogram and probability density of CD321 expression in Normal Karyotype patients from datasets covering a broad variety of known AML subtypes (Bullinger, L., et al. (2004). Use of gene-expression profiling to identify prognostic subclasses in adult acute myeloid leukemia. The New England journal of medicine 350, 1605-1616; Chao, M., Seita, J., and Weissman, I. (2008). Establishment of a Normal Hematopoietic and Leukemia Stem Cell Hierarchy. Cold Spring Harbor Symposia on Quantitative Biology, 1-12; Kharas, 2010). Threshold scores are marked with dashed line. Survival analysis of CD321^{high} and CD321^{low} patients from all patients (E), (H) and only patients with Normal Karyotype (F), (I) suggests that prediction of survival based on CD321 expression in bulk blasts works better in patients with Normal Karyotype.

FIG. 8. Difference in cancer lineage differentiation between the Type 1 and Type 2 AML patients. (A) Diagram showing cancer clone differentiation in Type 1 and Type 2 patients including the existence of cytokine feedback loops suggested by analysis of the culture supernatants from sorted CD321^{high} and CD36^{high} subsets from Type 1 patients. (B, C) Gene set enrichment analysis (Genepattern, Broad Institute) of cells that do and do not respond to G-CSF in Type 1 AML patients.

FIG. 9. (A) CD321 expression levels in unsorted blasts reveal two subgroups of patients. Patient data from a public dataset (Metzeler et al., supra) were scored by the value of projection onto first dimension of PCA (done on natural RMA gene expression values read from four probe sets corresponding to CD321 gene on U133A Affymetrix microarray). A threshold score of 2000 was chosen to distinguish these two groups of patients. (B) AML patients with overall high CD321 expression have less chance of survival. Survival analysis (Kaplan-Meier estimator) of CD321^{high} and CD321^{low} patients suggests that with a high probability CD321^{high} patients have low chance of survival. (C) Incorporating NPM1 status increases the robustness of CD321 based survival prediction. Survival analysis (Kaplan-Meier estimator) done only on NPM1-positive patients shows more robust survival stratification.

FIG. 10. (A) Two types of patients are present in an independent 2004 cohort. Expression of 500 genes differentially expressed in the PBMC of selected AML patients from 2004 cohort and levels of expression of the same genes in various blood cell subsets taken from a previously published study (Novershtern, N., et al. (2011). Densely interconnected transcriptional circuits control cell states in human hematopoiesis. Cell 144, 296-309). (B) Signaling threshold predicts average differentiation status of unsorted blasts in 2004 cohort. Bar graph showing the percentage of cells responding to G-CSF in selected AML patients from 2004 cohort. Red line indicates 13.5 signaling threshold separating Type 1 from Type 2 AML patients analyzed by subset sorting in this study (C) Percentages of cells responding to activation by selected cytokines in 2004 cohort.

FIG. 11. Type 1 and Type 2 AML are similar to Group2 and Group1 of Normal Karyotype AML as defined in Bullinger et al., supra. Gene expression in bulk Type 1 and Type 2 patients was estimated by sum of expression in GRC and NGRC multiplied by the percentage of these cells in the bulk samples. 6200 genes differentially expressed in samples from between different AML patients (listed as provided by Bullinger et al., supra) were matched against the genes differentially expression between Type 1 and Type 2 bulk samples. Heatmap of 1200 genes selected based on highest average log 2 fold between Type 1 and Type 2 samples are shown on the left panel. Right heatmap shows the corresponding genes from Bullinger et al., supra. Note the coincident high expression in Type 1 AML and Group 2 samples and similarly the Type 2 AML and Group 1 samples.

### DETAILED DESCRIPTION OF THE INVENTION

Methods, compositions, and kits are provided for the use of JAM-A in diagnosing and treating leukemia. These methods, compositions, and kits find many uses, for example in diagnosing an individual with a leukemia, classifying a leukemia, providing a prognosis to an individual with a leukemia, treating an individual with a leukemia, screening candidate agents for the ability to treat a leukemia, and basic research to better understand the molecular and cellular basis of leukemia. These and other objects, advantages, and features of the invention will become apparent to those persons skilled in the art upon reading the details of the compositions and methods as more fully described below.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

The terms “cancer”, “neoplasm”, “tumor”, and “carcinoma”, are used interchangeably herein to refer to cells which exhibit relatively autonomous growth, so that they exhibit an aberrant growth phenotype characterized by a significant loss of control of cell proliferation. In general, cells of interest for detection or treatment in the present application include precancerous (e.g., benign), malignant, pre-metastatic, metastatic, and non-metastatic cells. Detection of cancerous cells is of particular interest. The term “normal” as used in the context of “normal cell,” is meant to refer to a cell of an untransformed phenotype or exhibiting a morphology of a non-transformed cell of the tissue type being examined. “Cancerous phenotype” generally refers to any of a variety of biological phenomena that are characteristic of a cancerous cell, which phenomena can vary with the type of cancer. The cancerous phenotype is generally identified by abnormalities in, for example, cell growth or proliferation (e.g., uncontrolled growth or proliferation), regulation of the cell cycle, cell mobility, cell-cell interaction, or metastasis, etc.

The terms “hematological malignancy”, “hematological tumor”, and “hematological cancer” are used interchangeably and in the broadest sense herein and refer to all stages and all forms of cancer arising from cells of the hematopoietic system.

By “leukemia” it is meant a class of hematological malignancy, i.e., a type of cancer of the blood or bone marrow. Leukemias are characterized by an abnormal increase of immature white blood cells, or “blasts”, particularly in the peripheral blood. By “blast cells” and “blasts” it is meant the immature precursors of lymphocytes (lymphoblasts), granulocytes (myeloblasts), monocytes (monoblasts), thrombocytes (megakaryoblast) or erythrocytes (proerythroblasts). Blast cells do not normally appear in peripheral blood, but will do so in leukemia. For example, myeloblasts and lymphoblasts are normally found in the bone marrow, but in acute myelogenous leukemia (AML) and acute lymphoblastic leukemia (ALL), myeloblasts and lymphoblasts, respectively, proliferate uncontrollably and are found in large numbers in the peripheral blood. Blast cells can be recognized by their large size and primitive nuclei (i.e. the nuclei contain nucleoli). In addition, they may be identified and isolated from a blood sample, e.g. peripheral blood, by flow cytometry based on their mid-level expression of the cell surface marker CD45 and their high Side Scatter (SSC) activity. In contrast, mature hematopoietic cells, e.g. lymphocytes, monocytes, macrophages, neutrophils, NK cells, dendritic cells, etc., may be identified and isolated from a blood sample by flow cytometry based on their high-level expression of the cell surface marker CD45. By ‘blast crisis’, it is meant a patient with too many immature precursors, e.g. abnormal myeloid cells, in the peripheral blood. By “blast counts” it is meant the number of immature precursors, e.g. abnormal myeloid cells, in the peripheral blood. Blast counts are useful for defining disease status or therapy effect, where an efficient treatment will yield a <5% blast count.

“Diagnosis” as used herein generally includes a prediction of a subject's susceptibility to a disease or disorder, determination as to whether a subject is presently affected by a disease or disorder, prognosis of a subject affected by a disease or disorder (e.g., identification of cancerous states, stages of cancer, likelihood that a patient will die from the cancer), classification of the subject's disease or disorder into a subtype of the disease or disorder, prediction of a subject's responsiveness to treatment for the disease or disorder (e.g., positive response, a negative response, no response at all to, e.g., allogeneic hematopoietic stem cell transplantation, chemotherapy, radiation therapy, antibody therapy, small molecule compound therapy) and use of therametrics (e.g., monitoring a subject's condition to provide information as to the effect or efficacy of therapy).

“Prognosis” as used herein generally includes a prediction of the course of disease progression and/or disease outcome, and may include the expected duration, the function, and a description of the course of the disease Examples of prognostic predictions include prognoses of long-term survival, overall survival (OS), relapse-free survival (RFS) and/or event-free survival (EFS).

By “long-term” survival it is meant survival for a particular time period, e.g., for at least 3 years, more preferably for at least 5 years, taking into consideration the median age at which patients are diagnosed with leukemia and the median survival of all patients with leukemia.

By “Overall Survival” or “OS” it is meant the time (in years) is measured from diagnosis, study entry, or early randomization (depending on the study design) to death from any cause. OS is defined for all patients of a trial; patients not known to have died at last follow-up are censored on the date at which they were last known to be alive. Overall survival is a term that denotes the chances of staying alive for a group of individuals suffering from a cancer. It denotes the percentage of individuals in the group who are likely to be alive after a particular duration of time.

By “Relapse-Free Survival” or “RFS” it is meant the time (in years) measured from diagnosis, study entry, or early randomization (depending on the study design) to first hematological malignancy recurrence. RFS is defined only for patients achieving complete remission, whether with complete blood count recovery (“CR”, e.g. a blood count comprising less than 5% bone marrow blasts, the absence of blasts with Auer rods, the absence of extramedullary disease, an absolute neutrophil count of greater than 1.0×10⁹/L (1000 μL); a platelet count of greater than 100×10⁹/L (100 000/μL), and an independence from red cell transfusions) or without complete blood count recovery (“CRi”, e.g. complete remission except for residual neutropenia (<1.0×10⁹/L [1000/μL]) or thrombocytopenia (<100×10⁹/L [100 000/μL])). RFS is measured from the date of achievement of a remission until the date of relapse or death from any cause; patients not known to have relapsed or died at last follow-up are censored on the date at which they were last examined.

By “Event-Free Survival” or “EFS” it is meant the time (in years) measured from diagnosis, study entry, or early randomization (depending on the study design) to the first subsequent event associated with the disease, e.g. complications from the disease, first malignancy recurrence, or death. EFS is defined for all patients of a trial, and is measured from the date of entry into a study to the date of induction treatment failure, or relapse from CR or CRi, or death from any cause; patients not known to have any of these events are censored on the date they were last examined.

The term “risk classification” means a level of risk (or likelihood) that a subject will experience a particular clinical outcome. A subject may be classified into a risk group or classified at a level of risk based on the methods of the present disclosure, e.g. high, medium, or low risk. A “risk group” is a group of subjects or individuals with a similar level of risk for a particular clinical outcome.

By “expression level” it is meant the level of JAM-A gene product, e.g. the normalized value determined for the RNA expression level of JAM-A or for the expression level of a polypeptide encoded by the JAM-A gene.

The term “gene product” or “expression product” are used herein to refer to the RNA transcription products (RNA transcripts, e.g. mRNA, an unspliced RNA, a splice variant mRNA, a microRNA, and a fragmented RNA) of the gene, including mRNA, and the polypeptide translation products of such RNA transcripts. A gene product can be, for example, an unspliced RNA, an mRNA, a splice variant mRNA, a microRNA, a fragmented RNA, a polypeptide, a post-translationally modified polypeptide, a splice variant polypeptide, etc.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues.

The subject therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired, particularly humans.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

As summarized above, methods, compositions, and kits are provided for the use of JAM-A in diagnosing and treating leukemia. In further describing the invention, the subject methods of diagnosis are described first, followed by a description of the subject methods of treatment.

### Methods of Diagnosis

Aspects of the invention are directed to diagnosing leukemia in an individual. By “leukemia” it is meant a class of hematological malignancy characterized by an abnormal increase of immature white blood cells, or “blasts”, particularly in the peripheral blood. Blast cells, or “blasts” are the immature precursors of lymphocytes (lymphoblasts), granulocytes (myeloblasts), monocytes (monoblasts), thrombocytes (megakaryoblast) or erythrocytes (proerythroblasts). Blast cells do not normally appear in peripheral blood, but will do so in leukemia. For example, myeloblasts and lymphoblasts are normally found in the bone marrow, but in acute myelogenous leukemia (AML) and acute lymphoblastic leukemia (ALL), myeloblasts and lymphoblasts, respectively, proliferate uncontrollably and are found in large numbers in the peripheral blood. Examples of leukemias to which the subject methods may be applied include, without limitation, Acute myelogenous leukemia (AML), Acute lymphocytic leukemia (ALL), Chronic lymphocytic leukemia (CLL), Chronic myelogenous leukemia (CML), T-cell prolymphocytic leukemia (T-PLL), B-cell prolymphocytic leukemia (B-PLL), Chronic neutrophilic leukemia (CNL), Hairy cell leukemia (HCL), T-cell large granular lymphocyte leukemia (T-LGL), and Aggressive NK-cell leukemia.

As demonstrated in the examples below, the inventors of the present application have observed that a population of hematopoietic cells that, when found in elevated levels in an individual with leukemia, is associated with poor prognosis, express the cell surface protein JAM-A. Accordingly, in practicing methods of the invention, a patient sample is assayed to measure the expression of the JAM-A gene product. In other words, the amount of JAM-A protein or RNA in a blood sample, or the number of cells that express JAM-A protein or RNA in a blood sample, is detected to determine whether an individual has a leukemia, to classify a leukemia, to provide a prognosis for a leukemia, or to predict responsiveness of a leukemia to a treatment. JAM-A (Junctional Adhesion Molecule-A, also known as JAM-1, F11 receptor (F11R) or CD321) is a multifunctional cell surface protein that has multiple evolutionarily conserved structural features. JAM-A protein is expressed at tight junctions of endothelial and epithelial cells and on the surface of platelets and leukocytes. JAM-A signaling regulates a number of cellular activities, including, for example, migration, polarity, paracellular permeability, and proliferation. A discussion of JAM-A structure and its involvement in these and other cellular activities may be found in Severson et al. Structural determinants of Junctional Adhesion Molecule A (JAM-A) function and mechanisms of intracellular signaling. Curr Opin Cell Biol. 2009 October; 21(5):701-7; Mandell K J et al. The JAM family of proteins Adv Drug Deliv Rev. 2005 Apr. 25; 57(6):857-67; Naik T U, et al. Junctional adhesion molecules in angiogenesis. Front Biosci. 2008 Jan. 1; 13:258-62; and Weber C, et al. The role of junctional adhesion molecules in vascular inflammation. Nat Rev Immunol. 2007 June; 7(6):467-77; the full disclosures of which are incorporated herein by reference. The polypeptide sequence for JAM-A and the nucleic acid that encodes it may be found at Genbank Accession No. NM_{—}016946.4.

In some embodiments, the patient sample is a hematologic sample. Examples of hematologic samples include, without limitation, a peripheral blood sample, a bone marrow sample, a spleen biopsy, a lymph node biopsy, and the like, or a fraction thereof. A hematologic sample may be collected by any convenient method, e.g. blood draw, biopsy, etc. The sample may be freshly assayed or it may be stored and assayed at a later time. If the latter, the sample may be stored by any means known in the art to be appropriate in view of the method chosen for assaying protein expression, discussed further below. For example the sample may freshly cryopreserved, that is, cryopreserved without impregnation with fixative, e.g. at 4° C., at −20° C., at −60° C., at −80° C., or under liquid nitrogen. Alternatively, the sample may be fixed and preserved, e.g. at room temperature, at 4° C., at −20° C., at −60° C., at −80° C., or under liquid nitrogen, using any of a number of fixatives known in the art, e.g. alcohol, methanol, acetone, formalin, paraformaldehyde, etc.

The sample may be assayed as a whole sample, e.g. in crude form. Alternatively, the sample may be fractionated prior to analysis, e.g. by density gradient centrifugation, panning, magnetic bead sorting, fluorescence activated cell sorting (FACS), etc. to purify leukocytes or one or more fractions thereof, e.g. blast cells, thereby arriving at an enriched population of cells for analysis. The sample, i.e. the whole sample or the fraction(s) thereof, is then assayed to measure the expression levels of JAM-A. In some instances, as when the sample is a tissue biopsy that will be sectioned for analysis, the sample may be embedded in sectioning medium, e.g. OCT or paraffin. The sample is then sectioned, and one or more sections are then assayed to measure which cells express JAM-A and/or at what level.

In some instances, the number of JAM-A+ cells in the sample is measured, e.g. by detecting the staining intensity of cells stained with a probe that is specific for JAM-A. In other words, the number of cells that are positive for JAM-A are measured with a JAM-A specific probe. By “specific” and “specific binding” it is meant binding which occurs between such paired species as enzyme/substrate, receptor/ligand, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or ligand/receptor interaction. Antibodies to JAM-A (CD321) are commercially available; see, for example, the examples below.

It will be understood by those of skill in the art that the stated expression levels reflect detectable amounts of the marker protein on the cell surface. A cell that is negative for staining (the level of binding of a marker specific reagent is not detectably different from an isotype matched control) may still express minor amounts of the marker. And while it is commonplace in the art to refer to cells as “positive” or “negative” for a particular marker, actual expression levels are quantitative traits. The number of molecules on the cell surface can vary by several logs, yet still be characterized as “positive”.

The staining intensity of cells can be monitored by flow cytometry, where lasers detect the quantitative levels of fluorochrome (which is proportional to the amount of cell surface marker bound by specific reagents, e.g. antibodies). Flow cytometry, or FACS, can also be used to separate cell populations based on the intensity of binding to a specific reagent, as well as other parameters such as cell size and light scatter. Although the absolute level of staining may differ with a particular fluorochrome and reagent preparation, the data can be normalized to a control.

In order to normalize the distribution to a control, each cell is recorded as a data point having a particular intensity of staining. These data points may be displayed according to a log scale, where the unit of measure is arbitrary staining intensity. In one example, the brightest stained cells in a sample can be as much as 4 logs more intense than unstained cells. When displayed in this manner, it is clear that the cells falling in the highest log of staining intensity are bright, while those in the lowest intensity are negative. The “low” positively stained cells have a level of staining brighter than that of an isotype matched control, but is not as intense as the most brightly staining cells normally found in the population. An alternative control may utilize a substrate having a defined density of marker on its surface, for example a fabricated bead or cell line, which provides the positive control for intensity.

In some instances, the level of JAM-A expression in the sample is measured. By “expression level” it is meant the level of JAM-A gene product, e.g. the normalized value determined for the RNA expression level of JAM-A or for the expression level of a polypeptide encoded by the JAM-A gene. The term “gene product” or “expression product” are used herein to refer to the RNA transcription products (RNA transcripts, e.g. mRNA, an unspliced RNA, a splice variant mRNA, a microRNA, and a fragmented RNA) of the gene, including mRNA, and the polypeptide translation products of such RNA transcripts. A gene product can be, for example, an unspliced RNA, an mRNA, a splice variant mRNA, a microRNA, a fragmented RNA, a polypeptide, a post-translationally modified polypeptide, a splice variant polypeptide, etc.

For measuring protein levels, the amount or level of JAM-A polypeptide in the sample is determined, e.g., the protein/polypeptide encoded by the JAM-A. In some instances, the expression level of one or more additional proteins may also be measured, and the level of JAM-A expression compared to the level of the one or more additional proteins to provide a normalized value for JAM-A expression. Any convenient protocol for evaluating protein levels may be employed wherein the level of one or more proteins in the assayed sample is determined.

While a variety of different manners of assaying for protein levels are known in the art, one representative and convenient type of protocol for assaying protein levels is ELISA. In ELISA and ELISA-based assays, one or more antibodies specific for the proteins of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Following incubation, the antisera-contacted surface is washed so as to remove non-immunocomplexed material. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g. urease, peroxidase, or alkaline phosphatase, which will generate a color precipitate upon incubating with an appropriate chromogenic substrate. After such incubation with the second antibody and washing to remove unbound material, the amount of label is quantified, for example by incubation with a chromogenic substrate such as urea and bromocresol purple in the case of a urease label or 2,2′-azino-di-(3-ethyl-benzthiazoline)-6-sulfonic acid (ABTS) and H2O2, in the case of a peroxidase label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody. The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected in a manner most appropriate for the substrate being used, for example, by removing a bead or dipstick from a reservoir, emptying or diluting a reservoir such as a microtiter plate well, or rinsing a bead, particle, chromatograpic column or filter with a wash solution or solvent.

Alternatively, non-ELISA based-methods for measuring the levels of one or more proteins in a sample may be employed. Representative examples include but are not limited to western blotting, mass spectrometry, proteomic arrays, xMAP™ microsphere technology, immunohistochemistry, and flow cytometry.

A number of exemplary methods are also known in the art for measuring mRNA expression levels in a sample, include, without limitation, hybridization-based methods, e.g. northern blotting and in situ hybridization (Parker & Barnes, Methods in Molecular Biology 106:247-283 (1999)), RNAse protection assays (Hod, Biotechniques 13:852-854 (1992)), and PCR-based methods (e.g. reverse transcription PCR(RT-PCR) (Weis et al., Trends in Genetics 8:263-264 (1992)).

For measuring mRNA levels, the starting material is typically total RNA or poly A+ RNA isolated from a suspension of cells, e.g. a peripheral blood sample a bone marrow sample, etc., or from a homogenized tissue, e.g. a homogenized biopsy sample, a homogenized paraffin- or OCT-embedded sample, etc. General methods for mRNA extraction are well known in the art and are disclosed in standard textbooks of molecular biology, including Ausubel et al., Current Protocols of Molecular Biology, John Wiley and Sons (1997). RNA isolation can also be performed using a purification kit, buffer set and protease from commercial manufacturers, according to the manufacturer's instructions. For example, RNA from cell suspensions can be isolated using Qiagen RNeasy mini-columns, and RNA from cell suspensions or homogenized tissue samples can be isolated using the TRIzol reagent-based kits (Invitrogen), MasterPure™ Complete DNA and RNA Purification Kit (EPICENTRE™, Madison, Wis.), Paraffin Block RNA Isolation Kit (Ambion, Inc.) or RNA Stat-60 kit (Tel-Test).

A variety of different manners of measuring mRNA levels are known in the art, e.g. as employed in the field of differential gene expression analysis. One representative and convenient type of protocol for measuring mRNA levels is array-based gene expression profiling. Such protocols are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively.

Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions, and unbound nucleic acid is then removed. The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

The resultant pattern of hybridized nucleic acid provides information regarding expression for each of the genes that have been probed, where the expression information is in terms of whether or not the gene is expressed and, typically, at what level, where the expression data, i.e., expression profile (e.g., in the form of a transcriptosome), may be both qualitative and quantitative.

Alternatively, non-array based methods for quantitating the level of one or more nucleic acids in a sample may be employed. These include those based on amplification protocols, e.g., Polymerase Chain Reaction (PCR)-based assays, including quantitative PCR, reverse-transcription PCR (RT-PCR), real-time PCR, and the like, e.g. TaqMan® RT-PCR, MassARRAY® System, BeadArray® technology, and Luminex technology; and those that rely upon hybridization of probes to filters, e.g. Northern blotting and in situ hybridization.

The resultant data provides information regarding expression of JAM-A, wherein the expression information is in terms of whether or not the gene is expressed and, typically, at what level and/or on what percentage of the cells of the population, and wherein the expression data may be both qualitative and quantitative. These data may be used to make a diagnosis of leukemia in an individual, to classify a leukemia as a Type 1 or Type 2 AML, to provide a prognosis for an individual with leukemia, or to predict the responsiveness of an individual to a leukemia treatment.

For example, as alluded to above and demonstrated in the examples below, the present inventors have observed that high expression levels of JAM-A in a hematologic sample is diagnostic of leukemia and predictive of a poor prognosis. By “high expression levels”, it is meant expression levels that are 2 to 3 fold greater or more than JAM-A levels from a normal patient sample, for example 4-fold greater, 5-fold greater, 6-fold greater, 8-fold greater, 10-fold greater or more than JAMA-A levels in a healthy patient sample. In some instances, the sample is a PBMC sample, e.g. an unstimulated PBMC sample. In some instances, the sample is a blast sample, e.g. a sample of blast cells from bone marrow, e.g. a sample of blast cells that have been stimulated with G-CSF. By a “poor prognosis”, it is meant that the patient has about a 40% chance of overall survival at least 10 months after diagnosis, or about a 25% chance of overall survival at least 20 months after diagnosis. In contrast, low or negligible expression of JAM-A in a peripheral blood sample suggests that the disease may be a hematological disorder other than leukemia, or that if the disease is leukemia, that the patient has a better prognosis, e.g. about a 65% chance of overall survival at least 10 months after diagnosis, or about a 55% chance of overall survival at least 20 months after diagnosis, or about a 50% chance of overall survival at least 30 months after diagnosis. Any convenient metric may be used to measure and convey a prognosis or a prediction of responsiveness to a therapy. For example, predictions may be made in terms of progression free survival (PFS), overall survival (OS), relapse-free survival (RFS) and/or event-free survival (EFS).

As another example, as also alluded to above and demonstrated in the examples below, the present inventors have identified two subtypes of acute myelogenous leukemia, Type 1 AML and Type 2 AML, which may be distinguished by measuring JAM-A expression levels in a sample or the number of cells expressing high levels of JAM-A. In Type 1 AML, the peripheral blood of the leukemia patient comprises a population of blast cells that comprise two subpopulations, a small population of peripheral blasts that are responsive to G-CSF (GRCs, approximately 0.5%-15% of the blast cells), and a large population of peripheral blasts that are non-responsive to G-CSF (NGRCs, approximately 85-99.5% of the blast cells). Furthermore GRCs and the NGRCs in this type of AML have different gene expression profiles, the GRCs having a gene expression profile resembling hematopoietic progenitor cells (a “primitive” profile, including high expression of JAM-A) and the NGRCs having a gene expression profile resembling differentiated cells of the myeloid lineage (a “myeloid” profile, including low/no expression of JAM-A, and high expression of LFA1 (CD11a/CD18), CD36, and CD1). In Type 2 AML, the peripheral blood of the leukemia patient again comprises a population of blast cells that comprise two subpopulations, but the representation of these subpopulations in the blast population are skewed relative to Type 1 AML: there are more peripheral blasts that are responsive to G-CSF (GRCs, about 15-50% of the blast cells) and less peripheral blasts that are non-responsive to G-CSF. Moreover, in Type 2 AML, both GRCs and the NGRCs have the same type of gene expression profile, this profile resembling that of hematopoietic progenitor cells (a “primitive” profile, including high expression of JAM-A). It is known in the art that patients with a relatively high number of peripheral blasts that respond to G-CSF (GRCs) have a poor prognosis. As such, patients classified by the subject methods as having Type 2 AML have a relatively poor prognosis as compared to patients classified as having Type 1 AML.

Accordingly, in some embodiments of the subject methods, a leukemia may classified as Type 1 or Type 2 AML by determining the level of expression in a hematologic sample, wherein a high level of expression is indicative of Type 2 AML. By “high expression levels”, it is meant expression levels that are 2 fold greater or more than JAM-A levels in a Type 1 AML sample, for example 3-fold greater, 4-fold greater, or 5-fold greater or more than JAMA-A levels in a healthy patient sample. In some embodiments, a leukemia may classified as Type 1 or Type 2 AML by determining the percentage of cells expressing JAM-A in the peripheral blood sample or cell population or fraction thereof, wherein a low percentage of JAM-A+ cells, e.g. 50% or less of CD45^{mid} blast cells, e.g. 45% or less, 40% or less, 35% or less, 30% or less, or 25% or less, is indicative of Type 1 AML, and a high percentage of cells, e.g. 60% or more of CD45^{mid} blast cells, e.g. 65% or more, 70% or more, 75% or more, usually 80% or more, 85% or more, sometimes 90% or more, for example 95% or more, is indicative of Type 2 AML. In some embodiments, a leukemia is classified as Type 1 or Type 2 AML by determining the percentage of cells expressing LFA in the peripheral blood sample, wherein a high percentage of LFA+ cells, e.g. 50% or more of CD45^{mid} blast cells, e.g. 60% or more of CD45^{mid} blast cells, e.g. 65% or more, 70% or more, 75% or more, usually 80% or more, 85% or more, sometimes 90% or more, for example 95% or more, is indicative of Type 1 AML, and a low percentage of LFA+ cells, e.g. 15% or less of CD45^{mid} blast cells, e.g. 12% or less, 8% or less, 5% or less is indicative of Type 2 AML. In some embodiments, the sample is a peripheral blood sample. In some embodiments, the sample is a peripheral blood mononuclear cell sample. In some embodiments, the sample is a population of blast cells.

In some embodiments, a leukemia is classified as Type 1 or Type 2 AML by measuring the number of GRCs in a peripheral blood sample or cell population or fraction thereof, e.g. a PBMC sample, or a blast sample. Additionally or alternatively, a leukemia may be classified as Type 1 or Type 2 AML by measuring the number of NGRCs in a peripheral blood sample or a fraction thereof, e.g. a blast sample. In some embodiments, the sample is a peripheral blood sample. In some embodiments, the sample is a peripheral blood mononuclear cell sample. In some embodiments, the sample is a population of blast cells. In some embodiments, the cell population is stimulated with G-CSF, e.g. as described in the examples section below, and the number of GRCs and/or NGRCs is determined in the stimulated population to arrive at the classification of Type 1 or Type 2 AML. As discussed above and as demonstrated in the examples below, a percentage of GRCs in a blast sample of about 0.5-15%, and a percentage of NGRCs in a blast sample of about 85-100%, is indicative of Type 1 AML, whereas a percentage of GRCs in a blast sample of about 20%-50%, and a percentage of NGRCs in a blast sample of about 50-80% is indicative of Type 2 AML. Any convenient method may be used to measure the number of GRCs and/or NGRCs in a peripheral blood sample or fraction thereof. For example, and as demonstrated below, GRCs are readily identifiable from NGRCs by the activation of intracellular proteins in GRCs (and lack thereof in NGRCs) following stimulation with G-CSF, e.g. Stat-3 and Stat-5 phosphorylation. GRCs also typically have a higher colony formation capacity than NGRCs. NGRCs express a number of cytokines, e.g. G-CSF, GM-CSF, IL-1α, IL-1β, IL-4, IL-5, IL-6, and SCF, at levels that are at least 10-fold greater than GRCs.

In some instances, the resultant data are used directly to diagnose leukemia, to classify a type of leukemia, or to provide a prognosis to an individual that has a leukemia. In other instances, the measurements are compared to measurements from one or more control samples, e.g. a hematologic sample from a human subject who does not have a leukemia (i.e., a negative control), or a hematological sample from a human subject that has a leukemia, or that has a particular subtype of leukemia (i.e., a positive control, e.g. Type 1 AML or Type 2 AML), and a diagnosis, classification of a type of leukemia, or a prognosis is made based upon the comparison.

The subject diagnostic methods may be used alone or in combination with other clinical methods for patient stratification known in the art, e.g. age, cytogenetics, the presence of certain molecular mutations, the altered expression levels of particular genes, e.g. IL2RA and MSI2, and the like, to provide a diagnosis, a prognosis, or a prediction of responsiveness to therapy. For example, for AML, known clinical prognostic factors associated with favorable outcome include cytogenetic mutations such as t(15;17)PML/RARα, t(8;21)AML1/ETO, 11q23, and inv(16)CBFβ/MYH11, or molecular mutations in FLT3 (e.g., FLT3-ITD, FLT3-D835), NPM1, EVI1, or cEBPα; clinical prognostic factors that have been associated with an intermediate outcome include Normal karyotype, and the cytogenetic mutations +8, +21, +22, del(7q), and del(9q); and clinical prognostic factors that have been associated with an adverse outcome include the cytogenetic mutations del(5q), 11q23, t(6; 9), t(9; 22), abnormal 3q, complex cytogenetics, and elevated expression levels of IL2Ra and/or MSI2. For example, and as demonstrated below, detecting the presence of an NPM1 AML mutation, e.g. by genotyping the patient for an NPM1 AML mutation, improves the statistical significance of prognoses made using the subject methods.

In some embodiments, providing an evaluation of a subject for a leukemia, i.e., a diagnosis, a prognosis, or a prediction of responsiveness to therapy, includes generating a written report that includes the artisan's assessment of the subject's current state of health i.e. a “diagnosis assessment”, of the subject's prognosis, i.e. a “prognosis assessment”, and/or of possible treatment regimens, i.e. a “treatment assessment”. Thus, a subject method may further include a step of generating or outputting a report providing the results of a diagnosis assessment, a prognosis assessment, or treatment assessment, which report can be provided in the form of an electronic medium (e.g., an electronic display on a computer monitor), or in the form of a tangible medium (e.g., a report printed on paper or other tangible medium).

A “report,” as described herein, is an electronic or tangible document which includes report elements that provide information of interest relating to a diagnosis assessment, a prognosis assessment, and/or a treatment assessment and its results. A subject report can be completely or partially electronically generated. A subject report includes at least a diagnosis assessment, i.e. a diagnosis as to whether a subject has a leukemia; or a prognosis assessment, i.e. a prediction of the likelihood that a patient with a leukemia will have a leukemia-attributable death or progression, including recurrence, metastatic spread, and drug resistance; or a treatment assessment, i.e. a prediction as to the likelihood that a leukemia patient will have a particular clinical response to treatment, and/or a suggested course of treatment to be followed. A subject report can further include one or more of: 1) information regarding the testing facility; 2) service provider information; 3) subject data; 4) sample data; 5) an assessment report, which can include various information including: a) test data, where test data can include i) the expression level of the JAM-A gene, ii) the percentage of cells expressing the JAM-A gene, b) reference values employed, if any; 6) other features.

The report may include information about the testing facility, which information is relevant to the hospital, clinic, or laboratory in which sample gathering and/or data generation was conducted. This information can include one or more details relating to, for example, the name and location of the testing facility, the identity of the lab technician who conducted the assay and/or who entered the input data, the date and time the assay was conducted and/or analyzed, the location where the sample and/or result data is stored, the lot number of the reagents (e.g., kit, etc.) used in the assay, and the like. Report fields with this information can generally be populated using information provided by the user.

The report may include information about the service provider, which may be located outside the healthcare facility at which the user is located, or within the healthcare facility. Examples of such information can include the name and location of the service provider, the name of the reviewer, and where necessary or desired the name of the individual who conducted sample gathering and/or data generation. Report fields with this information can generally be populated using data entered by the user, which can be selected from among pre-scripted selections (e.g., using a drop-down menu). Other service provider information in the report can include contact information for technical information about the result and/or about the interpretive report.

The report may include a subject data section, including subject medical history as well as administrative subject data (that is, data that are not essential to the diagnosis, prognosis, or treatment assessment) such as information to identify the subject (e.g., name, subject date of birth (DOB), gender, mailing and/or residence address, medical record number (MRN), room and/or bed number in a healthcare facility), insurance information, and the like), the name of the subject's physician or other health professional who ordered the susceptibility prediction and, if different from the ordering physician, the name of a staff physician who is responsible for the subject's care (e.g., primary care physician).

The report may include a sample data section, which may provide information about the biological sample analyzed, such as the source of biological sample obtained from the subject (e.g. blood, type of tissue, etc.), how the sample was handled (e.g. storage temperature, preparatory protocols) and the date and time collected. Report fields with this information can generally be populated using data entered by the user, some of which may be provided as pre-scripted selections (e.g., using a drop-down menu).

The report may include an assessment report section, which may include information generated after processing of the data as described herein. The interpretive report can include a prognosis of the likelihood that the patient will have a cancer-attributable death or progression. The interpretive report can include, for example, results of the gene expression analysis, methods used to calculate the expression levels, and interpretation, i.e. prognosis. The assessment portion of the report can optionally also include a Recommendation(s). For example, where the results indicate that the subject will be responsive to induction chemotherapy, the recommendation can include a recommendation that a bone marrow transplant be performed with induction chemotherapy to follow.

It will also be readily appreciated that the reports can include additional elements or modified elements. For example, where electronic, the report can contain hyperlinks which point to internal or external databases which provide more detailed information about selected elements of the report. For example, the patient data element of the report can include a hyperlink to an electronic patient record, or a site for accessing such a patient record, which patient record is maintained in a confidential database. This latter embodiment may be of interest in an in-hospital system or in-clinic setting. When in electronic format, the report is recorded on a suitable physical medium, such as a computer readable medium, e.g., in a computer memory, zip drive, CD, DVD, etc.

It will be readily appreciated that the report can include all or some of the elements above, with the proviso that the report generally includes at least the elements sufficient to provide the analysis requested by the user (e.g., a diagnosis, a prognosis, or a prediction of responsiveness to a therapy).

### Methods of Treatment

Aspects of the invention are directed to treating leukemia in an individual. The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. Aspects of the treatment methods described herein typically comprise the use of a JAM-A-based therapeutic, e.g. an agent that binds to JAM-A and targets a JAM-A+ cell for for depletion (e.g. by separation, by death), and agent that binds to JAM-A and modulates JAM-A signaling, etc. In some embodiments, the treatment comprises removing JAM-A+ cells from the individual's blood, i.e. depleting the blood of JAM-A+ cells. In some embodiments, the treatment comprises administering to the individual an effective amount of a JAM-A-specific binding agent conjugated to a therapeutic moiety, e.g. to deplete target JAM-A+ cells, or alter their activity. In some embodiments, the treatment comprises administering to the individual an effective amount of an agent that modulates the activity of the JAM-A signaling pathway. In some embodiments, a combination of these treatments may be applied, that is, two or more of the methods described herein may be employed to treat an individual for leukemia. In some embodiments, the treatment methods may be performed concurrent with or after treatment regimens known in the art.

As discussed above, the inventors of the present application have observed that G-CSF responsive hematopoietic cells (GRCs), which are associated with poor prognosis when found in elevated levels in an individual with leukemia, express the cell surface protein JAM-A. As such, in one embodiment, the method of treating leukemia comprises removing, or depleting, GRCs from the individual's blood.

In some embodiments, GRCs are removed, or depleted, from the blood ex vivo, i.e. outside the body of the individual, The population that remains after removing the GRCs is then returned to the individual, e.g. to reconstitute the individual's hematopoietic system/blood. In such embodiments, a hematologic sample, i.e. a population of leukocytes, referred to hereafter as “the subject initial population” is obtained from the individual. The individual may be any mammalian species, e.g. human, primate, equine, bovine, porcine, canine, feline, etc. More usually, it is from human. The subject initial population may include fresh or frozen cells, which may be from a neonate, a juvenile or an adult. For example, cells, e.g. blood cells, e.g. leukocytes, may be harvested by apheresis, leukocytapheresis, density gradient separation, etc. The cells may be used immediately, or they may be stored, frozen, for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cells.

In some embodiments, the GRCs are depleted from the subject initial population by contacting the subject initial population with an agent that binds specifically to JAM-A, and isolating, or separating, the agent-bound JAM-A+ cells from the rest of the population. JAM-A+ cells may be separated from a population by any convenient separation technique. For example, the JAM-A+ cells may be separated from the subject initial population by affinity separation techniques. Techniques for affinity separation may include magnetic separation (MACS) using magnetic beads coated with an affinity reagent, affinity chromatography, “panning” with an affinity reagent attached to a solid matrix, e.g. plate, cytotoxic agents joined to an affinity reagent or used in conjunction with an affinity reagent, e.g. complement and cytotoxins, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide). Any technique may be employed which is not unduly detrimental to the viability of the JAM-A-negative cells.

To separate the JAM-A+ cells from JAM-A− cells by affinity separation techniques, JAM-A-negative cells may be selected for by contacting the population with affinity reagents that specifically recognize and selectively bind markers that are not expressed on JAM-A+ cells, for example, myeloid markers (e.g. LFA1 (CD11a), CD36, or CD1), lymphocyte markers (B220, CD3), NK markers (e.g. NK1.1), and markers of other non-blast cell types. By “selectively bind” is meant that the molecule binds preferentially to the target of interest or binds with greater affinity to the target than to other molecules. For example, an antibody will bind to a molecule comprising an epitope for which it is specific and not to unrelated epitopes. In some embodiments, the affinity reagent may be an antibody, i.e. an antibody that is specific for JAM-A. In some embodiments, the affinity reagent may be a specific receptor or ligand for JAM-A, e.g. a soluble LFA1 polypeptide or peptide thereof, a T cell receptor specific for JAM-A, and the like. In some embodiments, multiple affinity reagents specific for JAM-A may be used.

Antibodies and T cell receptors that find use as affinity reagents may be monoclonal or polyclonal, and may be produced by transgenic animals, immunized animals, immortalized human or animal B-cells, cells transfected with DNA vectors encoding the antibody or T cell receptor, etc. The details of the preparation of antibodies and their suitability for use as specific binding members are well-known to those skilled in the art. Of particular interest is the use of labeled antibodies as affinity reagents. Conveniently, these antibodies are conjugated with a label for use in separation. Labels include magnetic beads, which allow for direct separation; biotin, which can be removed with avidin or streptavidin bound to a support; fluorochromes, which can be used with a fluorescence activated cell sorter; or the like, to allow for ease of separation of the particular cell type. Fluorochromes that find use include phycobiliproteins, e.g. phycoerythrin and allophycocyanins, fluorescein and Texas red. Frequently each antibody is labeled with a different fluorochrome, to permit independent sorting for each marker.

The subject initial population of leukocytes are contacted with the affinity reagent(s) and incubated for a period of time sufficient to bind the available cell surface antigens. The incubation will usually be at least about 5 minutes and usually less than about 60 minutes. It is desirable to have a sufficient concentration of antibodies in the reaction mixture, such that the efficiency of the separation is not limited by lack of antibody. The appropriate concentration is determined by titration, but will typically be a dilution of antibody into the volume of the cell suspension that is about 1:50 (i.e., 1 part antibody to 50 parts reaction volume), about 1:100, about 1:150, about 1:200, about 1:250, about 1:500, about 1:1000, about 1:2000, or about 1:5000. The medium in which the cells are suspended will be any medium that maintains the viability of the cells. A preferred medium is phosphate buffered saline containing from 0.1 to 0.5% BSA or 1-4% goat serum. Various media are commercially available and may be used according to the nature of the cells, including Dulbecco's Modified Eagle Medium (dMEM), Hank's Basic Salt Solution (HBSS), Dulbecco's phosphate buffered saline (dPBS), RPMI, Iscove's medium, PBS with 5 mM EDTA, etc., frequently supplemented with fetal calf serum, BSA, HSA, goat serum etc.

The cells in the contacted population that become labeled by the affinity reagent, i.e. the JAM-A⁺ cells, are removed from the population by any convenient affinity separation technique, e.g. as described above or as known in the art. Following separation, the separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum.

Compositions that are highly enriched for JAM-A-negative cells are achieved in this manner. As such, a substantially pure population of JAM-A-negative cells, and hence nonresponsive to G-CSF, will be prepared, where by “substantially pure” it is meant having at least about 80%, about 85%, or about 90% of the cells of the population be nonresponsive to G-CSF, more usually at least 95% or more of the population being nonresponsive to G-CSF, e.g. 95%, 98%, and up to 100% of the population. Substantially pure cell compositions that are prepared in this manner may be returned to the individual's blood stream, i.e. to reconstitute the individual's blood.

In some embodiments, the GRCs are depleted from the subject initial population by contacting the subject initial population ex vivo with an agent that binds specifically to JAM-A and is conjugated to a therapeutic moiety that promotes cell death. Such agents may also be used to deplete GRCs in vivo, i.e. by administering to the individual the JAM-A binding agent conjugated to a therapeutic moiety that promotes the death of the cell or alters the activity of the cell. By “therapeutic moiety”, or “TM”, it is meant a polypeptide, small molecule or nucleic acid composition that confers a therapeutic activity upon a composition. Examples of therapeutic moieties include cytotoxins, e.g. small molecule compounds, protein toxins, and radiosensitizing moieties, i.e. radionuclides etc. that are intrinsically detrimental to a cell; agents that alter the activity of a cell, e.g. small molecules, peptide mimetics, cytokines, chemokines; and moieties that target a cell for antibody-dependent cell-mediated cytotoxicity (ADCC) or complement dependent cytotoxicity (CDC)-dependent death, e.g. the Fc component of immunoglobulin. In some embodiments, an agent that specifically binds to JAM-A, that is, a “JAM-A-specific binding agent,” e.g. an LFA peptide, a JAM-A-specific antibody, etc. is conjugated to one or more therapeutic moieties such as polypeptides, drugs, radionucleotides, or toxins. In other words, the subject composition comprises a functional moiety conjugated to the JAM-A-specific binding agent. See, e.g., PCT publications WO 92/08495; WO 91/14438; WO 89/12624; U.S. Pat. No. 5,314,995; and EP 396,387. Therapeutic moieties include, without limitation, moieties that promote cell death and moieties that alter cellular activity.

Examples of moieties that promote cell death include cytotoxic agents, i.e. a cytotoxin, e.g., a cytostatic or cytocidal small molecule, a polypeptide agent or a radioactive metal ion. A cytotoxin or cytotoxic agent includes any agent that is detrimental to cells. Examples include paclitaxol, cytochalasin B, gramicidin D, ethidium bromide, emetine, mitomycin, etoposide, tenoposide, vincristine, vinblastine, colchicine, doxorubicin, daunorubicin, dihydroxy anthracenedione, mitoxantrone, mithramycin, actinomycin D, 1-dehydrotestosterone, glucocorticoids, procaine, tetracaine, lidocaine, propranolol, and puromycin and analogs or homologues thereof. Cytotoxic agents also include proteins, peptides, or polypeptides possessing a cytotoxic biological activity, e.g., toxins such as abrin, ricin A, pseudomonas exotoxin, cholera toxin, and diphtheria toxin. Cytotoxic agents also include radioactive metal ions, i.e. radionuclides, such as alpha-emitters, e.g. Bismuth-213, Radium-226, Lead-212, Actinium-225, and Astatine-211, and β-emitters, e.g. Iodide-131, Yttrium-90, Rhenium-188, Lutetium-177, Copper-67 and Copper-64, and macrocyclic chelators useful for conjugating radiometal ions, e.g. 131In, 131L, 131Y, 131Ho, 131Sm, to polypeptides or any of those listed supra. Macrocyclic chelators can be attached to the antibody via a linker molecule, e.g. as described in Denardo et al., 1998, Clin Cancer Res. 4(10):2483-90; Peterson et al., 1999, Bioconjug. Chem. 10(4):553-7; and Zimmerman et al., 1999, Nucl. Med. Biol. 26(8):943-50, each incorporated by reference in their entireties.

Moieties that promote cell death also include moieties that target a cell for antibody-dependent cell-mediated cytotoxicity (ADCC), antibody dependent cell-mediated phagocytosis (ADCP), or complement dependent cytotoxicity (CDC, also known as complement-mediated cytolysis, or CMC), e.g. the Fc component of immunoglobulin. See, for example, Raghavan et al., 1996, Annu Rev Cell Dev Biol 12:181-220; Ghetie et al., 2000, Annu Rev Immunol 18:739-766; Ravetch et al., 2001, Annu Rev Immunol 19:275-290). To assess ADCC activity of a molecule of interest, an in vitro ADCC assay may be performed. Useful effector cells for such assays include peripheral blood mononuclear cells (PBMC) and Natural Killer (NK) cells. Alternatively or additionally, ADCC activity of the molecule of interest may be assessed in vivo, e.g., in an animal model such as that disclosed in Clynes et al. PNAS (USA) 95:652-656 (1998). All FcγRs bind the same region on Fc, at the N-terminal end of the Cγ2 domain and the preceding hinge, which region may be utilized as a functional moiety for the purposes of the invention. An overlapping but separate site on Fc serves as the interface for the complement protein C1q. In the same way that Fc/FcγR binding mediates ADCC and ADCP, Fc/C1q binding mediates complement dependent cytotoxicity (CDC). A site on Fc between the Cγ2 and Cγ3 domains mediates interaction with the neonatal receptor FcRn, the binding of which recycles endocytosed antibody from the endosome back to the bloodstream

As used herein, an Fc fusion is synonymous with the terms “immunoadhesin”, “Ig fusion”, “Ig chimera”, and “receptor globulin” as used in the art (Chamow et al., 1996, Trends Biotechnol 14:52-60; Ashkenazi et al., 1997, Curr Opin Immunol 9:195-200). An Fc fusion combines the Fc region of an immunoglobulin with the JAM-A-specific binding agent, for example. See for example U.S. Pat. Nos. 5,766,883 and 5,876,969, both of which are expressly incorporated by reference.

Therapeutic moieties other than those that promote cell death would include agents that alter the activity of a cell. Such therapeutic agents include, but are not limited to, cytokines, chemokines, antimetabolites (e.g., methotrexate, 6-mercaptopurine, 6-thioguanine, cytarabine, 5-fluorouracil decarbazine), alkylating agents (e.g., mechlorethamine, thiotepa chlorambucil, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Other functional moieties suitable for conjugation to JAM-A-specific binding agents of the present application include imaging moieties. As discussed above, an imaging moiety is a non-cytotoxic agent that can be used to locate and, optionally, visualize cells, e.g. cells that have been targeted by compositions of the subject application. For example, fluorescent dyes may be used as an imaging moiety. In another example, radioactive agents that are non-cytotoxic may also be an imaging moiety. An imaging moiety may require the addition of a substrate for detection, e.g. horseradish peroxidase (HRP), β-galactosidase, luciferase, and the like. Alternatively, an imaging moiety may provide a detectable signal that does not require the addition of a substrate for detection, e.g. a fluorophore or chromophore dye, e.g. Alexa Fluor 488® or Alexa Fluor 647®, or a protein that comprises a fluorophore or chromophore, e.g. GFP, RFP, dsRED, phiYFP, etc. and mutants thereof.

Techniques for conjugating functional moieties to polypeptides, e.g. JAM-A-specific binding agents, are well known in the art, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982).

Functional moieties are typically bound to JAM-A-specific binding agents of the subject compositions by covalent interactions. In some embodiments, a linker may be used, where the linker may be any moiety that can be used to link the JAM-A-specific binding agent polypeptide to the functional moiety. In some embodiments, the linker is a cleavable linker. The use of a cleavable linker enables the moiety linked to the JAM-A-specific binding agent to be released from the JAM-A-specific binding agent once absorbed by the cell, and transported to the cell body. The cleavable linker may be cleavable by a chemical agent, by an enzyme, due to a pH change, or by being exposed to energy. Examples of forms of energy that may be used include light, microwave, ultrasound, and radiofrequency.

In certain applications, it may be desirable to release the functional moiety, particularly where the moiety is a therapeutic moiety, once the compound has entered the cell, resulting in a release of the moiety. Accordingly, in one variation, the linker L is a cleavable linker. This enables the moiety M to be released from the compound once in a cell. This may be desirable when, for example, the functional moiety is a therapeutic moiety which has a greater therapeutic effect when separated from the JAM-A-specific binding agent. For example, the therapeutic moiety may have a better ability to be absorbed by an intracellular component of the cell when separated from the JAM-A-specific binding agent. Accordingly, it may be necessary or desirable to separate the therapeutic moiety from the JAM-A-specific binding agent so that the therapeutic moiety can enter the intracellular compartment.

In some embodiments, an effective amount of the JAM-A-specific binding agent is provided to deliver a functional moiety to a G-CSF responsive cell (GRC), e.g. a therapeutic moiety and/or an imaging moiety, e.g. in an individual with a leukemia and comprising GRC peripheral blood mononuclear cells (PBMCs). In such embodiments, an effective amount will be the amount required to achieve therapeutic or imaging efficacy by the functional moiety.

For example, in some embodiments, the functional moiety is a therapeutic moiety that is cytotoxic. An effective amount of a composition comprising a cytotoxic moiety will be the amount sufficient to promote cell death selectively in the cells targeted by the JAM-A-specific binding agent to which the cytotoxic moiety is fused, including GRCs. In some instances, the effect amount of functional moiety is well known; e.g. radionuclides are typically delivered in the range of 10-30 cGy/h, the regimen depending on the half-life of the radioisotope. In other instances, the effective amount can be readily determined by one of ordinary skill in the art using any convenient method known in the art for assaying for cell death, e.g. TUNEL staining, Annexin staining, propidium iodide uptake, etc. It will be understood by those of skill in the art that an initial dose may be administered for such periods of time, followed by maintenance doses, which, in some cases, will be at a reduced dosage.

As another example, in some embodiments, the functional moiety is a therapeutic moiety that targets a cell for ADCC or CDC. An effective amount of a composition comprising a moiety that targets a cell for ADCC or CDC will be the amount sufficient to promote ADCC or CDC selectively in the cells targeted by the JAM-A-specific binding agent to which the Fc moiety is fused. The effective amount can be readily determined by one of ordinary skill in the art using any convenient method known in the art for assaying ADCC and CDC.

As mentioned above, methods for depleting GRCs may be used to treat an individual for leukemia. These methods also find other uses, for example, in research, e.g. to better understand the molecular and cellular basis of leukemia. The GRC-depleting agent may be delivered ex vivo, e.g. by isolating leukocytes as described above, and contacting the cells ex vivo with the agent, and then returning the leukocytes to the blood stream of the individual; or it may be delivered in vivo, by administering the agent to the individual. Formulations, dosages and methods for administering agents to an individual are described in greater detail below.

In some embodiments, leukemia may be treated by contacting JAM-A+ cells ex vivo or in vivo with an agent that modulates JAM-A pathway signaling. By modulating, it is meant the activity of the JAM-A pathway is inhibited, i.e. reduced, suppressed, decreased, attenuated or antagonized, or promoted, i.e. activated, stimulated, elevated, or enhanced. Any agent that modulates JAM-A pathway signaling, i.e. a “JAM-A antagonist” or “JAM-A agonist”, may be used in the subject method. For example, JAM-A+ leukocytes may be contacted with an agent that antagonizes JAM-A activity directly, i.e. by reducing JAM-A expression levels or by physically interacting with JAM-A protein, etc. As another example, JAM-A+ leukocytes may be contacted with an agent that antagonizes JAM-A signaling indirectly, i.e. by modulating the activity of proteins that modulate JAM-A activity, e.g. LFA1 (CD11a). As another example, JAM-A+ leukocytes may be contacted with an agent that promotes JAM-A activity, i.e. by increasing JAM-A expression levels or by physically interacting with JAM-A protein. As another example, JAM-A+ leukocytes may be contacted with an agent that promotes JAM-A signaling indirectly, i.e. by modulating the activity of proteins that modulate JAM-A activity, e.g. LFA1 (CD11a). Agents that modulate the activity of any of these proteins would modulate the activity of JAM-A, and as such would find use as JAM-A modulators in the subject methods.

In some embodiments, the modulator of JAM-A signaling is a polypeptide or peptide. For example, the modulator may be a soluble native LFA1 polypeptide, peptide or variant thereof that binds to JAM-A and blocks binding of LFA1 to JAM-A, thereby antagonizing JAM-A signaling, or that binds to JAM-A and activates JAM-A. By “native polypeptide” it is meant a polypeptide found in nature. By “variant” it is meant a mutant of the native polypeptide having less than 100% sequence identity with the native sequence, e.g. 65%, 70%, 75%, or 80% or more identity, such as 85%, 90%, or 95% or more identity, for example, 98% or 99% identity with the full length native polypeptide. As another example, the modulator may be a polypeptide or peptide inhibitor or activator of an endogenous activator of JAM-A, for example, a polypeptide or peptide antagonist or agonist of LFA1, e.g. a native JAM-A polypeptide, peptide or variant thereof that binds to LFA1. In some embodiments, the modulator is an antibody, for example, in the case of an antagonist, a non-activating JAM-A-specific antibody or an antibody that is specific for an endogenous activator of JAM-A such as LFA1, or in the case of an agonist, an activating JAM-A specific antibody. In some embodiments, the JAM-A modulator is a nucleic acid, for example, a nucleic acid that encodes a polypeptide or peptide antagonist or agonist, a microRNA that upregulates or downregulates JAM-A (e.g. miR-145 (Götte M, et al. miR-145-dependent targeting of junctional adhesion molecule A and modulation of fascin expression are associated with reduced breast cancer cell motility and invasiveness. Oncogene. 2010, 29(50):6569-80)) or an siRNA that is specific for JAM-A or a JAM-A inhibitor. In some embodiments, the JAM-A modulator is a small molecule that binds directly to JAM-A and inhibits JAM-A activity, or that binds directly to and inhibits the activity of an endogenous activator of JAM-A, e.g. a small molecule inhibitor of LFA1.

In some embodiments of the subject methods, the JAM-A modulator is administered to the individual in an effective amount. Biochemically speaking, an effective amount or effective dose of JAM-A modulator is an amount of modulator that will alter JAM-A pathway signaling in a cell by 30% or more, 40% or more, 50% or more, sometimes 60% or more, 70% or more, or 80% or more, or in some instances 90% or more, e.g. 95% or 100%. In other words JAM-A pathway signaling will be altered about 0.5-fold or more, 1-fold or more, 2-fold or more, 5-fold or more, 8-fold or more, 10-fold or more, 20-fold or more, 50-fold or more, or 100-fold or more. The amount of modulation of a cell's activity by a JAM-A modulator can be determined by a number of ways known to one of ordinary skill in the art of molecular biology.

In a clinical sense, an effective dose of a JAM-A modulator is the dose that, when administered for a suitable period of time, usually at least about one week, and maybe about two weeks, or more, up to a period of about 4 weeks, 8 weeks, or longer will evidence an alteration the symptoms associated with undesired activity of the JAM-A signaling pathway. For example, an effective dose of a JAM-A modulator may be the dose that when administered for a suitable period of time, usually at least about one week, and may be about two weeks, or more, up to a period of about 4 weeks, 8 weeks, or longer will slow, halt or reverse proliferation of JAM-A+ blast cells in a patient suffering from leukemia. It will be understood by those of skill in the art that an initial dose may be administered for such periods of time, followed by maintenance doses, which, in some cases, will be at a reduced dosage.

As discussed above, some of the subject methods of the invention rely on the use of JAM-A-based agents, i.e. JAM-A therapeutic agents, for example, agents that modulate JAM-A signaling, JAM-A-specific binding agents conjugated to a therapeutic moiety, etc. In some instances, these agents are polypeptides or peptides. Such polypeptides/peptides may optionally be fused to a peptide domain that increases solubility of the product. The domain may be linked to the polypeptide through a defined protease cleavage site, e.g. a TEV sequence, which is cleaved by TEV protease. The linker may also include one or more flexible sequences, e.g. from 1 to 10 glycine residues. In some embodiments, the cleavage of the fusion protein is performed in a buffer that maintains solubility of the product, e.g. in the presence of from 0.5 to 2 M urea, in the presence of polypeptides and/or polynucleotides that increase solubility, and the like. Domains of interest include endosomolytic domains, e.g. influenza HA domain; and other polypeptides that aid in production, e.g. IF2 domain, GST domain, GRPE domain, and the like. The polypeptide may be formulated for improved stability. For example, the peptides may be PEGylated, where the polyethyleneoxy group provides for enhanced lifetime in the blood stream.

Additionally or alternatively, the JAM-A-based therapeutic agent may be myristoylated or fused to a polypeptide permeant domain to promote uptake by the cell. A number of permeant domains are known in the art and may be used in the non-integrating polypeptides of the present invention, including peptides, peptidomimetics, and non-peptide carriers. For example, a permeant peptide may be derived from the third alpha helix of Drosophila melanogaster transcription factor Antennapaedia, referred to as penetratin. As another example, the permeant peptide comprises the HIV-1 tat basic region amino acid sequence, which may include, for example, amino acids 49-57 of naturally-occurring tat protein. Other permeant domains include poly-arginine motifs, for example, the region of amino acids 34-56 of HIV-1 rev protein, nona-arginine, octa-arginine, and the like. (See, for example, Futaki et al. (2003) Curr Protein Pept Sci. 2003 April; 4(2): 87-96; and Wender et al. (2000) Proc. Natl. Acad. Sci. U.S.A 2000 Nov. 21; 97(24):13003-8; published U.S. Patent applications 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002). The site at which the fusion is made may be selected in order to optimize the biological activity, secretion or binding characteristics of the polypeptide. The optimal site will be determined by routine experimentation.

The JAM-A-based therapeutic agent may be produced by eukaryotic cells or by prokaryotic cells, it may be further processed by unfolding, e.g. heat denaturation, DTT reduction, etc. and may be further refolded, using methods known in the art. Alternatively, the JAM-A-based therapeutic agent may be prepared by in vitro synthesis, using conventional methods as known in the art. Various commercial synthetic apparatuses are available, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like.

Modifications of interest that do not alter primary sequence include chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Also included in the subject invention are JAM-A-based therapeutic agents that have been modified using ordinary molecular biological techniques and synthetic chemistry so as to improve their resistance to proteolytic degradation or to optimize solubility properties or to render them more suitable as a therapeutic agent. Analogs of such polypeptides include those containing residues other than naturally occurring L-amino acids, e.g. D-amino acids or non-naturally occurring synthetic amino acids. D-amino acids may be substituted for some or all of the amino acid residues.

If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The JAM-A-based therapeutic agent may be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

As mention above, the JAM-A-based therapeutic agent may be a nucleic acid. mRNA encoding JAM-A-based therapeutic agents may be provided to cells using well-developed transfection techniques; see, e.g. Angel and Yanik (2010) PLoS ONE 5(7): e11756, and the commercially available TransMessenger® reagents from Qiagen, Stemfect™ RNA Transfection Kit from Stemgent, and TransIT®-mRNA Transfection Kit from Mirus Bio LLC. See also Beumer et al. (2008) Efficient gene targeting in Drosophila by direct embryo injection with zinc-finger nucleases. PNAS 105(50):19821-19826. Alternatively, nucleic acids encoding JAM-A-based therapeutic agents may be provided on DNA vectors. Many vectors, e.g. plasmids, cosmids, minicircles, phage, viruses, etc., useful for transferring nucleic acids into target cells are available. The vectors comprising the nucleic acid(s) may be maintained episomally, e.g. as plasmids, minicircle DNAs, viruses such cytomegalovirus, adenovirus, etc., or they may be integrated into the target cell genome, through homologous recombination or random integration, e.g. retrovirus-derived vectors such as MMLV, HIV-1, ALV, etc.

Vectors may be provided directly to JAM-A⁺ cells. In other words, the cells are contacted with vector comprising the nucleic acid encoding the JAM-A-based therapeutic agent such that the vector is taken up by the cells. Methods for contacting cells with nucleic acid vectors that are plasmids, such as electroporation, calcium chloride transfection, and lipofection, are well known in the art. For viral vector delivery, the cells are contacted with viral particles comprising the nucleic acid encoding the JAM-A-based therapeutic agent. Retroviruses, for example, lentiviruses, are particularly suitable to the method of the invention. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line, the appropriate packaging cell line being selected to ensure that the cells are targeted by the packaged viral particles. Methods of introducing the retroviral vectors comprising the nucleic acid encoding the JAM-A-based therapeutic agent into packaging cell lines and of collecting the viral particles that are generated by the packaging lines are well known in the art.

Vectors used for providing the nucleic acids encoding a JAM-A-based therapeutic agent to the subject cells will typically comprise suitable promoters for driving the expression, that is, transcriptional activation, of the nucleic acid of interest. In other words, the nucleic acid of interest will be operably linked to a promoter. This may include ubiquitously acting promoters, for example, the CMV-β-actin promoter, or inducible promoters, such as promoters that are active in particular cell populations or that respond to the presence of drugs such as tetracycline. By transcriptional activation, it is intended that transcription will be increased above basal levels in the target cell by at least about 10 fold, by at least about 100 fold, more usually by at least about 1000 fold. In addition, vectors used for providing the JAM-A-based therapeutic agent to the subject cells may include nucleic acid sequences that encode for selectable markers in the target cells, so as to identify cells that have taken up the JAM-A-based therapeutic agent.

As also mentioned above, the JAM-A-based therapeutic agent may be provided to cells as a small molecule. Small molecule compounds may be dissolved in water or alcohols or solvents such as DMSO or DMF, and diluted into water or an appropriate buffer prior to being provided to cells.

In in vivo methods, an effective amount of a JAM-A-based therapeutic agent is administered to a subject in need thereof. For example, JAM-A-based therapeutic agents of specific interest are those that inhibit proliferation and/or metastasis of leukemia cells or induce the death of leukemia cells in a host when the JAM-A-based therapeutic agent is administered in an effective amount. The amount administered varies depending upon the goal of the administration, the health and physical condition of the individual to be treated, age, the taxonomic group of individual to be treated (e.g., human, non-human primate, primate, etc.), the degree of resolution desired, the formulation of the JAM-A-based therapeutic composition, the treating clinician's assessment of the medical situation, and other relevant factors. It is expected that the amount will fall in a relatively broad range that can be determined through routine trials. For example, the amount of JAM-A-based therapeutic agent employed to inhibit cancer metastasis is not more than about the amount that could otherwise be irreversibly toxic to the subject (i.e., maximum tolerated dose). In other cases the amount is around or even well below the toxic threshold, but still in an immunoeffective concentration range, or even as low as threshold dose.

Individual doses are typically not less than an amount required to produce a measurable effect on the subject, and may be determined based on the pharmacokinetics and pharmacology for absorption, distribution, metabolism, and excretion (“ADME”) of the JAM-A-based therapeutic agent's by-products, and thus based on the disposition of the composition within the subject. This includes consideration of the route of administration as well as dosage amount, which can be adjusted for topical (applied directly where action is desired for mainly a local effect), enteral (applied via digestive tract for systemic or local effects when retained in part of the digestive tract), or parenteral (applied by routes other than the digestive tract for systemic or local effects) applications. For instance, administration of the JAM-A-based therapeutic agent may be topical or via injection, e.g. intravenous, intramuscular, or intratumoral injection or a combination thereof.

The JAM-A-based therapeutic agent may be administered by infusion or by local injection, e.g. by infusion at a rate of about 50 mg/h to about 400 mg/h, including about 75 mg/h to about 375 mg/h, about 100 mg/h to about 350 mg/h, about 150 mg/h to about 350 mg/h, about 200 mg/h to about 300 mg/h, about 225 mg/h to about 275 mg/h. Exemplary rates of infusion can achieve a desired therapeutic dose of, for example, about 0.5 mg/m2/day to about 10 mg/m2/day, including about 1 mg/m2/day to about 9 mg/m2/day, about 2 mg/m2/day to about 8 mg/m2/day, about 3 mg/m2/day to about 7 mg/m2/day, about 4 mg/m2/day to about 6 mg/m2/day, about 4.5 mg/m2/day to about 5.5 mg/m2/day. Administration (e.g, by infusion) can be repeated over a desired period, e.g., repeated over a period of about 1 day to about 5 days or once every several days, for example, about five days, over about 1 month, about 2 months, etc. It also can be administered prior, at the time of, or after other therapeutic interventions, such as surgical intervention to remove cancerous cells. The JAM-A-based therapeutic agent can also be administered as part of a combination therapy, in which at least one of an immunotherapy, a cancer chemotherapy or a radiation therapy is administered to the subject (as described in greater detail below).

Disposition of the JAM-A-based therapeutic agent and its corresponding biological activity within a subject is typically gauged against the fraction of JAM-A-based therapeutic agent present at a target of interest. For example, once administered, a JAM-A-based therapeutic agent can accumulate with a glycoconjugate or other biological target that concentrates the material in cancer cells and cancerous tissue. Thus dosing regimens in which the JAM-A-based therapeutic agent is administered so as to accumulate in a target of interest over time can be part of a strategy to allow for lower individual doses. This can also mean that, for example, the dose of JAM-A-based therapeutic agent that are cleared more slowly in vivo can be lowered relative to the effective concentration calculated from in vitro assays (e.g., effective amount in vitro approximates mM concentration, versus less than mM concentrations in vivo).

As an example, the effective amount of a dose or dosing regimen can be gauged from the IC50 of a given JAM-A-based therapeutic agent for inhibiting cell migration. By “IC50” is intended the concentration of a drug required for 50% inhibition in vitro. Alternatively, the effective amount can be gauged from the EC50 of a given JAM-A-based therapeutic agent concentration. By “EC50” is intended the plasma concentration required for obtaining 50% of a maximum effect in vivo. In related embodiments, dosage may also be determined based on ED50 (effective dosage).

In general, with respect to the JAM-A-based therapeutic agent of the present disclosure, an effective amount is usually not more than 200× the calculated IC50. Typically, the amount of an JAM-A-based therapeutic agent that is administered is less than about 200×, less than about 150×, less than about 100× and many embodiments less than about 75×, less than about 60×, 50×, 45×, 40×, 35×, 30×, 25×, 20×, 15×, 10× and even less than about 8× or 2× than the calculated IC50. In one embodiment, the effective amount is about 1× to 50× of the calculated IC50, and sometimes about 2× to 40×, about 3× to 30× or about 4× to 20× of the calculated IC50. In other embodiments, the effective amount is the same as the calculated IC50, and in certain embodiments the effective amount is an amount that is more than the calculated IC50.

An effect amount may not be more than 100× the calculated EC50. For instance, the amount of a JAM-A-based therapeutic agent that is administered is less than about 100×, less than about 50×, less than about 40×, 35×, 30×, or 25× and many embodiments less than about 20×, less than about 15× and even less than about 10×, 9×, 9×, 7×, 6×, 5×, 4×, 3×, 2× or 1× than the calculated EC50. The effective amount may be about 1× to 30× of the calculated EC50, and sometimes about 1× to 20×, or about 1× to 10× of the calculated EC50. The effective amount may also be the same as the calculated EC50 or more than the calculated EC50. The IC50 can be calculated by inhibiting cell proliferation and/or cell migration/invasion in vitro. The procedure can be carry out by methods known in the art or as described in the examples below.

Effective amounts of dose and/or dose regimen can readily be determined empirically from assays, from safety and escalation and dose range trials, individual clinician-patient relationships, as well as in vitro and in vivo assays such as those described herein and illustrated in the Experimental section, below. For example, a concentration used for carrying out the subject method in mice ranges from about 1 mg/kg to about 25 mg/kg based on the body weight of the mice. Based on this data, an example of a concentration of the JAM-A-based therapeutic agent that can be employed in human may range about 0.083 mg/kg to about 2.08 mg/kg. Other dosage may be determined from experiments with animal models using methods known in the art (Reagan-Shaw et al. (2007) The FASEB Journal 22:659-661).

The JAM-A-based therapeutic agent can be incorporated into a variety of formulations. More particularly, the JAM-A-based therapeutic agent may be formulated into pharmaceutical compositions by combination with appropriate pharmaceutically acceptable carriers or diluents.

Pharmaceutical preparations are compositions that include one or more JAM-A-based therapeutic agent present in a pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be lipids, e.g. liposomes, e.g. liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semi-solid, liquid or gaseous forms, such as tablets, capsules, powders, granules, ointments, solutions, suppositories, injections, inhalants, gels, microspheres, and aerosols. As such, administration of the JAM-A-based therapeutic agent can be achieved in various ways, including transdermal, intradermal, oral, buccal, rectal, parenteral, intraperitoneal, intradermal, intracheal, etc., administration. The active agent may be systemic after administration or may be localized by the use of regional administration, intramural administration, or use of an implant that acts to retain the active dose at the site of implantation. The active agent may be formulated for immediate activity or it may be formulated for sustained release.

For some conditions, particularly central nervous system conditions, it may be necessary to formulate agents to cross the blood-brain barrier (BBB). One strategy for drug delivery through the blood-brain barrier (BBB) entails disruption of the BBB, either by osmotic means such as mannitol or leukotrienes, or biochemically by the use of vasoactive substances such as bradykinin. The potential for using BBB opening to target specific agents to brain tumors is also an option. A BBB disrupting agent can be co-administered with the therapeutic compositions of the invention when the compositions are administered by intravascular injection. Other strategies to go through the BBB may entail the use of endogenous transport systems, including Caveolin-1 mediated transcytosis, carrier-mediated transporters such as glucose and amino acid carriers, receptor-mediated transcytosis for insulin or transferrin, and active efflux transporters such as p-glycoprotein. Active transport moieties may also be conjugated to the therapeutic compounds for use in the invention to facilitate transport across the endothelial wall of the blood vessel. Alternatively, drug delivery of therapeutics agents behind the BBB may be by local delivery, for example by intrathecal delivery, e.g. through an Ommaya reservoir (see e.g. U.S. Pat. Nos. 5,222,982 and 5,385,582, incorporated herein by reference); by bolus injection, e.g. by a syringe, e.g. intravitreally or intracranially; by continuous infusion, e.g. by cannulation, e.g. with convection (see e.g. US Application No. 20070254842, incorporated here by reference); or by implanting a device upon which the agent has been reversably affixed (see e.g. US Application Nos. 20080081064 and 20090196903, incorporated herein by reference).

For inclusion in a medicament, the JAM-A-based therapeutic agent may be obtained from a suitable commercial source. As a general proposition, the total pharmaceutically effective amount of the JAM-A-based therapeutic agent administered parenterally per dose will be in a range that can be measured by a dose response curve.

JAM-A-based therapies, i.e. preparations of JAM-A-based therapeutic agent(s) to be used for therapeutic administration, may be sterile. Sterility is readily accomplished by filtration through sterile filtration membranes (e.g., 0.2 μm membranes). Therapeutic compositions generally are placed into a container having a sterile access port, for example, an intravenous solution bag or vial having a stopper pierceable by a hypodermic injection needle. The JAM-A-based therapies may be stored in unit or multi-dose containers, for example, sealed ampules or vials, as an aqueous solution or as a lyophilized formulation for reconstitution. As an example of a lyophilized formulation, 10-mL vials are filled with 5 ml of sterile-filtered 1% (w/v) aqueous solution of compound, and the resulting mixture is lyophilized. The infusion solution is prepared by reconstituting the lyophilized compound using bacteriostatic Water-for-Injection. Alternatively, the JAM-A-based therapeutic agent may be formulated into lotions for topical administration.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The nucleic acids or polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapies that exhibit large therapeutic indices are preferred.

The data obtained from cell culture and/or animal studies can be used in formulating a range of dosages for humans. The dosage of the active ingredient typically lines within a range of circulating concentrations that include the ED50 with low toxicity. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

Administration of a JAM-A based therapeutic agent can include administration as a part of a therapeutic regimen that may or may not be in conjunction with additional standard anti-cancer therapeutics, including but not limited to immunotherapy, chemotherapeutic agents and surgery (e.g., as those described further below).

In addition, therapeutic administration of the JAM-A based therapeutic agent can also be post-therapeutic treatment of the subject with an anti-cancer therapy, where the anti-cancer therapy can be, for example, surgery, radiation therapy, administration of chemotherapeutic agents, and the like. Cancer therapy using JAM-A based therapeutic agents of the present disclosure can also be used in combination with immunotherapy. In other examples, the JAM-A based therapeutic agent can be administered in combination with one or more chemotherapeutic agents (e.g., cyclophosphamide, doxorubicin, vincristine and prednisone (CHOP)), and/or in combination with radiation treatment and/or in combination with surgical intervention (e.g., pre- or post-surgery to remove a tumor). Where the JAM-A based therapeutic agent is used in connection with surgical intervention, the JAM-A based therapeutic agent can be administered prior to, at the time of, or after surgery to remove cancerous cells, and may be administered systemically or locally at the surgical site. The JAM-A based therapeutic agent alone or in combinations described above can be administered systemically (e.g., by parenteral administration, e.g., by an intravenous route) or locally (e.g., at a local tumor site, e.g., by intratumoral administration (e.g., into a solid tumor, into an involved lymph node in a lymphoma or leukemia), administration into a blood vessel supplying a solid tumor, etc.).

Any of a wide variety of cancer therapies can be used in combination with the JAM-A based therapies described herein. Such cancer therapies include surgery (e.g., surgical removal of cancerous tissue), radiation therapy, bone marrow transplantation, chemotherapeutic treatment, biological response modifier treatment, and certain combinations of the foregoing. Of particular interest are therapies for the treatment of leukemias.

Radiation therapy includes, but is not limited to, X-rays or gamma rays that are delivered from either an externally applied source such as a beam, or by implantation of small radioactive sources.

Chemotherapeutic agents are non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones. Agents that act to reduce cellular proliferation are known in the art and widely used.

Such agents include alkylating agents, such as nitrogen mustards, nitrosoureas, ethylenimine derivatives, alkyl sulfonates, and triazenes, including, but not limited to, mechlorethamine, cyclophosphamide (CYTOXAN™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (TAXOL®), docetaxel (TAXOTERE®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (TAXOL®), TAXOL® derivatives, docetaxel (TAXOTERE®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and ZOLADEX®. Estrogens stimulate proliferation and differentiation, therefore compounds that bind to the estrogen receptor are used to block this activity. Corticosteroids may inhibit T cell proliferation.

Other chemotherapeutic agents include metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc. Other anti-proliferative agents of interest include immunosuppressants, e.g. mycophenolic acid, thalidomide, desoxyspergualin, azasporine, leflunomide, mizoribine, azaspirane (SKF 105685); IRESSA® (ZD 1839, 4-(3-chloro-4-fluorophenylamino)-7-methoxy-6-(3-(4-morpholinyl)propoxy)quinazoline); etc.

“Taxanes” include paclitaxel, as well as any active taxane derivative or pro-drug. “Paclitaxel” (which should be understood herein to include analogues, formulations, and derivatives such as, for example, docetaxel, TAXOL, TAXOTERE (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art.

Paclitaxel should be understood to refer to not only the common chemically available form of paclitaxel, but analogs and derivatives (e.g., TAXOTERE™ docetaxel, as noted above) and paclitaxel conjugates (e.g., paclitaxel-PEG, paclitaxel-dextran, or paclitaxel-xylose).

In the treatment of some individuals in accordance with the method of the present disclosure, it may be desirable to use a high dose regimen in conjunction with a rescue agent for non-malignant cells. In such treatment, any agent capable of rescue of non-malignant cells can be employed, such as citrovorum factor, folate derivatives, or leucovorin. Such rescue agents are well known to those of ordinary skill in the art. Rescue agents include those which do not interfere with the ability of the present inventive compounds to modulate cellular function.

### Screening Methods

The discoveries described herein provide a useful system for screening candidate agents for the ability to treat leukemia, e.g. AML. To that end, it has been shown that a subpopulation of hematopoietic cells in a leukemia patient that is associated with the worst prognosis (G-CSF-responsive cells, or “GRCs”) expresses high levels of JAM-A. Accordingly, screening candidate agents for those that inhibit proliferation, inhibit metastasis, or promote apoptosis in cells expressing JAM-A should identify agents that will be useful in treating leukemia, e.g. AML.

For example, in screening assays for biologically active agents, cells expressing JAM-A are contacted with a candidate agent of interest and the effect of the candidate agent on the viability, proliferation rate, and/or metastatic potential of the JAM-A+ cells is assessed by monitoring one or more output parameters. Parameters are quantifiable components of cells, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values. Thus, for example, one such method may comprise contacting a cell that expresses JAM-A with the candidate agent; and comparing the parameter to the parameter in a cell that expresses JAM-A but was not contacted with the candidate agent, wherein a difference in the parameter in the cell contacted with the candidate agent indicates that the candidate agent will modulate JAM-A+ cell viability, proliferation, and/or metastasis.

One example of an output parameter that may be quantified when screening for agents that modulate cell viability, proliferation, and/or metastasis would be an output parameter that is reflective of an apoptotic state, such as the amount of DNA fragmentation, the amount of cell blebbing, the amount of phosphatidylserine on the cell surface as visualized by Annexin V staining, and the like; and/or an output parameter that is reflective of the viability of the culture, e.g. the number of cells in the culture, the uptake of BrdU in the culture, rate of expansion of the culture. Other output parameters could include those that are reflective of the function of the cells in the culture, e.g. the cytokines and chemokines produced by the cells, the rate of chemotaxis of the cells, the phagocytic activity of the cells, etc. In some instances, one parameter is measured. In some instances, multiple parameters are measured.

Cells useful for screening include any cell that expresses JAM-A and becomes activated in the presence of a JAM-A modulator, e.g. LFA1 polypeptide, LFA1 peptide, or an activating fragment thereof, a JAM-A antibody, e.g. JAM-A activating or inactivating antibody, etc. For example, the cell may be a cell that endogenously expresses JAM-A, e.g. a leukemic blast cell, e.g. as described herein. As another example, the cell may be a cell line that expresses JAM-A, i.e. either expresses JAM-A in its native state, or is genetically modified to ectopically express JAM-A. As another example, the cell may be acutely cultured from an individual that has a leukemia, e.g. AML.

In some instances, the method further comprises contacting the cells with a JAM-A modulator in an amount effective to modulate JAM-A signaling such that the cell begins to acquire a cancer phenotype, e.g. increased proliferation rates, increased colony formation, increased metastatic potential, etc. In some instances, this may be a JAM-A agonist. JAM-A agonists include, for example, soluble activating LFA1 polypeptide, cells that express LFA1, a JAM-A-specific antibody that activates JAM-A (“a JAM-A activating antibody”, e.g. the commercially available M.Ab.F11 antibody (BD Pharmingen)), etc. In some instances, this may be a JAM-A antagonist. JAM-A antagonists include, for example, LFA1 polypeptides that block activation of JAM-A, JAM-A-specific antibodies that block activation of JAM-A etc. In some instances, the method further comprises contacting the cells with cytokines that active JAM-A+ specific cells, e.g. as described in the examples section below. Cells may be contacted with these agents prior to contacting with the candidate agent, and/or concurrent with the candidate agent, and/or after contacting with the candidate agent.

Candidate agents of interest for screening include known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like.

Candidate agents include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof. Included are pharmacologically active drugs, genetically active molecules, etc. Compounds of interest include chemotherapeutic agents, hormones or hormone antagonists, etc. Exemplary of pharmaceutical agents suitable for this invention are those described in, “The Pharmacological Basis of Therapeutics,” Goodman and Gilman, McGraw-Hill, New York, N.Y., (1996), Ninth edition. Also included are toxins, and biological and chemical warfare agents, for example see Somani, S. M. (Ed.), “Chemical Warfare Agents,” Academic Press, New York, 1992).

Candidate agents of interest for screening also include nucleic acids, for example, nucleic acids that encode siRNA, shRNA, antisense molecules, or miRNA, or nucleic acids that encode polypeptides. Many vectors useful for transferring nucleic acids into target cells are available. The vectors may be maintained episomally, e.g. as plasmids, minicircle DNAs, virus-derived vectors such cytomegalovirus, adenovirus, etc., or they may be integrated into the target cell genome, through homologous recombination or random integration, e.g. retrovirus derived vectors such as MMLV, HIV-1, ALV, etc. Vectors may be provided directly to the subject cells. In other words, the pluripotent cells are contacted with vectors comprising the nucleic acid of interest such that the vectors are taken up by the cells.

Methods for contacting cells with nucleic acid vectors, such as electroporation, calcium chloride transfection, and lipofection, are well known in the art. Alternatively, the nucleic acid of interest may be provided to the subject cells via a virus. In other words, the pluripotent cells are contacted with viral particles comprising the nucleic acid of interest. Retroviruses, for example, lentiviruses, are particularly suitable to the method of the invention. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein to be incorporated into the capsid, this envelope protein determining the specificity of the viral particle for the cells. Envelope proteins are of at least three types, ecotropic, amphotropic and xenotropic. Retroviruses packaged with ecotropic envelope protein, e.g. MMLV, are capable of infecting most murine and rat cell types, and are generated by using ecotropic packaging cell lines such as BOSC23 (Pear et al. (1993) P.N.A.S. 90:8392-8396). Retroviruses bearing amphotropic envelope protein, e.g. 4070A (Danos et al, supra.), are capable of infecting most mammalian cell types, including human, dog and mouse, and are generated by using amphotropic packaging cell lines such as PA12 (Miller et al. (1985) Mol. Cell. Biol. 5:431-437); PA317 (Miller et al. (1986) Mol. Cell. Biol. 6:2895-2902); GRIP (Danos et al. (1988) PNAS 85:6460-6464). Retroviruses packaged with xenotropic envelope protein, e.g. AKR env, are capable of infecting most mammalian cell types, except murine cells. The appropriate packaging cell line may be used to ensure that the subject CD33+ differentiated somatic cells are targeted by the packaged viral particles. Methods of introducing the retroviral vectors comprising the nucleic acid encoding the reprogramming factors into packaging cell lines and of collecting the viral particles that are generated by the packaging lines are well known in the art.

Vectors used for providing nucleic acid of interest to the subject cells will typically comprise suitable promoters for driving the expression, that is, transcriptional activation, of the nucleic acid of interest. This may include ubiquitously acting promoters, for example, the CMV-b-actin promoter, or inducible promoters, such as promoters that are active in particular cell populations or that respond to the presence of drugs such as tetracycline. By transcriptional activation, it is intended that transcription will be increased above basal levels in the target cell by at least about 10 fold, by at least about 100 fold, more usually by at least about 1000 fold. In addition, vectors may include genes that must later be removed, e.g. using a recombinase system such as Cre/Lox, or the cells that express them destroyed, e.g. by including genes that allow selective toxicity such as herpesvirus TK, bcl-xs, etc

Candidate agents of interest for screening also include polypeptides. Such polypeptides may optionally be fused to a polypeptide domain that increases solubility of the product. The domain may be linked to the polypeptide through a defined protease cleavage site, e.g. a TEV sequence, which is cleaved by TEV protease. The linker may also include one or more flexible sequences, e.g. from 1 to 10 glycine residues. In some embodiments, the cleavage of the fusion protein is performed in a buffer that maintains solubility of the product, e.g. in the presence of from 0.5 to 2 M urea, in the presence of polypeptides and/or polynucleotides that increase solubility, and the like. Domains of interest include endosomolytic domains, e.g. influenza HA domain; and other polypeptides that aid in production, e.g. IF2 domain, GST domain, GRPE domain, and the like.

If the candidate polypeptide agent is being assayed for its ability to inhibit aggregation signaling intracellularly, the polypeptide may comprise the polypeptide sequences of interest fused to a polypeptide permeant domain. A number of permeant domains are known in the art and may be used in the non-integrating polypeptides of the present invention, including peptides, peptidomimetics, and non-peptide carriers. For example, a permeant peptide may be derived from the third alpha helix of Drosophila melanogaster transcription factor Antennapaedia, referred to as penetratin. As another example, the permeant peptide comprises the HIV-1 tat basic region amino acid sequence, which may include, for example, amino acids 49-57 of naturally-occurring tat protein. Other permeant domains include poly-arginine motifs, for example, the region of amino acids 34-56 of HIV-1 rev protein, nona-arginine, octa-arginine, and the like. (See, for example, Futaki et al. (2003) Curr Protein Pept Sci. 2003 April; 4(2): 87-96; and Wender et al. (2000) Proc. Natl. Acad. Sci. U.S.A 2000 Nov. 21; 97(24):13003-8; published U.S. Patent applications 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002).

If the candidate polypeptide agent is being assayed for its ability to inhibit aggregation signaling extracellularly, the polypeptide may be formulated for improved stability. For example, the peptides may be PEGylated, where the polyethyleneoxy group provides for enhanced lifetime in the blood stream. The polypeptide may be fused to another polypeptide to provide for added functionality, e.g. to increase the in vivo stability. Generally such fusion partners are a stable plasma protein, which may, for example, extend the in vivo plasma half-life of the polypeptide when present as a fusion, in particular wherein such a stable plasma protein is an immunoglobulin constant domain. In most cases where the stable plasma protein is normally found in a multimeric form, e.g., immunoglobulins or lipoproteins, in which the same or different polypeptide chains are normally disulfide and/or noncovalently bound to form an assembled multichain polypeptide, the fusions herein containing the polypeptide also will be produced and employed as a multimer having substantially the same structure as the stable plasma protein precursor. These multimers will be homogeneous with respect to the polypeptide agent they comprise, or they may contain more than one polypeptide agent.

The candidate polypeptide agent may be produced from eukaryotic produced by prokaryotic cells, it may be further processed by unfolding, e.g. heat denaturation, DTT reduction, etc. and may be further refolded, using methods known in the art. Modifications of interest that do not alter primary sequence include chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine. The polypeptides may have been modified using ordinary molecular biological techniques and synthetic chemistry so as to improve their resistance to proteolytic degradation or to optimize solubility properties or to render them more suitable as a therapeutic agent. Analogs of such polypeptides include those containing residues other than naturally occurring L-amino acids, e.g. D-amino acids or non-naturally occurring synthetic amino acids. D-amino acids may be substituted for some or all of the amino acid residues.

The candidate polypeptide agent may be prepared by in vitro synthesis, using conventional methods as known in the art. Various commercial synthetic apparatuses are available, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like. Alternatively, the candidate polypeptide agent may be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

In some cases, the candidate polypeptide agents to be screened are antibodies. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. The specific or selective fit of a given structure and its specific epitope is sometimes referred to as a “lock and key” fit. The archetypal antibody molecule is the immunoglobulin, and all types of immunoglobulins, IgG, IgM, IgA, IgE, IgD, etc., from all sources, e.g. human, rodent, rabbit, cow, sheep, pig, dog, other mammal, chicken, other avians, etc., are considered to be “antibodies.” Antibodies utilized in the present invention may be either polyclonal antibodies or monoclonal antibodies. Antibodies are typically provided in the media in which the cells are cultured.

Candidate agents may be obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Candidate agents are screened for biological activity by adding the agent to at least one and usually a plurality of cell samples, usually in conjunction with cells not contacted with the agent. The change in parameters in response to the agent is measured, and the result evaluated by comparison to reference cultures, e.g. in the presence and absence of the agent, obtained with other agents, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

Significant progress in molecular techniques, morphological classification and karyotyping have revealed a variety of molecular lesions that underlie the diversity of phenotypes and therapy responses manifested by patients with acute myeloid leukemia (AML) (Bennett, J. M., et al. (1985). Proposed revised criteria for the classification of acute myeloid leukemia. A report of the French-American-British Cooperative Group. Annals of internal medicine 103, 620-625; Dohner, H., et al. (2010). Diagnosis and management of acute myeloid leukemia in adults: recommendations from an international expert panel, on behalf of the European LeukemiaNet. In Blood, pp. 453-474; Lowenberg, B., et al. (1999). Acute myeloid leukemia. The New England Journal of Medicine 341, 1051-1062). In some cases, such as of former M3 of AML recognized now as a separate Acute Promyelocytic Leukemia (Lowenberg, B., et al. (2003). Acute myeloid leukemia and acute promyelocytic leukemia. Hematology/the Education Program of the American Society of Hematology American Society of Hematology Education Program, 82-101), a specific retinoid acid-based therapy has been shown to produce clinical remission in approximately 90% of patients; in contrast, for no other type of AML has a treatment of similar efficiency been developed. Therefore improvement of leukemia diagnostics is an important on-going pursuit of clinical hematology (Lowenberg, B. (2008). Acute myeloid leukemia: the challenge of capturing disease variety. Hematology/the Education Program of the American Society of Hematology American Society of Hematology Education Program, 1-11). A diagnostically significant variability between individual cancer cells both at genetic level and in functional capacity can be observed in AML patients (Anderson, K., et al. (2010). Genetic variegation of clonal architecture and propagating cells in leukaemia. Nature 469, 356-361; Ding, L., et al. (2012). Clonal evolution in relapsed acute myeloid leukaemia revealed by whole-genome sequencing. Nature 481, 506-510; Duhrsen, U., et al. (1995). Role of mature leukemic cells in the amplification of leukemic stem cells in a murine model. International Journal of Cancer 60, 652-659; Guan, Y., et al. (2002). Polyclonal normal hematopoietic progenitors in patients with acute myeloid leukemia. Experimental hematology 30, 721-728; Notta et al., 2011). Studies on differences in repopulation activity of individual cancer cells has shown that AML is a hierarchically organized cancer (Bonnet, D., and Dick, J. E. (1997). Human acute myeloid leukemia is organized as a hierarchy that originates from a primitive hematopoietic cell. Nature Medicine 3, 730-737), and contributed to the development of the stem cell model of cancer, now a predominant paradigm in oncology (Clarke, M. F., and Fuller, M. (2006). Stem cells and cancer: two faces of eve. Cell 124, 1111-1115). According to the cancer stem hypothesis only a minority of cancer cells, which in case of leukemias are called leukemic stem cells (LSC), have a capacity to divide indefinitely and maintain the cancer lineage (Dick, J. E. (2005). Acute Myeloid Leukemia Stem Cells. Annals of the New York Academy of Sciences 1044, 1-5; Wang, J. C. Y., and Dick, J. E. (2005). Cancer stem cells: lessons from leukemia. Trends in Cell Biology 15, 494-501). Like normal hematopoietic stem cells, LSCs express high levels of drug transporters and are relatively quiescent, which renders them drug resistant (Misaghian, N., et al. (2009). Targeting the leukemic stem cell: the Holy Grail of leukemia therapy. Leukemia 23, 25-42). In the case of CD34 positive AML, the LSCs reside within the CD34+CD38− compartment (Chao et al., 2008). Although it has been shown that most common chemotherapy regimens remove the majority of leukemic blasts, patients with AML often relapse, suggesting that LSCs resist treatment. It was demonstrated that the abundance of stem and progenitor cells in peripheral blood correlates with prognosis (Berer, A., et al. (2003). Circulating myeloid colony-forming cells predict survival in myelodysplastic syndromes. Annals of hematology 82, 271-277; Sutherland, H., et al. (2001). Detection and clinical significance of human acute myeloid leukaemia progenitors capable of long-term proliferation in vitro. British Journal of Haematology 114, 296-306).

Recently microarray profiling has been successfully used as a diagnostic tool as well as a way to study the regulatory mechanisms underpinning the biology of leukemic cells (Valk, P. J. M., et al. (2005). Gene expression profiling in acute myeloid leukemia. Current Opinion in Hematology 12, 76-81). It was shown to precisely distinguish major classes of leukemia (Armstrong, S. A., et al. (2001). MLL translocations specify a distinct gene expression profile that distinguishes a unique leukemia. Nature Genetics 30, 41-47; Golub, T. R., et al. (1999). Molecular classification of cancer: class discovery and class prediction by gene expression monitoring. Science (New York, N.Y.) 286, 531-537; Moos, P. J., et al. (2002). Identification of gene expression profiles that segregate patients with childhood leukemia. Clinical Cancer Research 8, 3118-3130) and in several cases to link the unique genetic abnormalities with specific expression profiles (Schoch, C., et al. (2002). Acute myeloid leukemias with reciprocal rearrangements can be distinguished by specific gene expression profiles. Proceedings Of The National Academy Of Sciences Of The United States Of America 99, 10008-10013; Verhaak, R. G. W., et al. (2009). Prediction of molecular subtypes in acute myeloid leukemia based on gene expression profiling. Haematologica 94, 131-134). Data from some studies indicate that gene expression can be used to stratify patients according to survival prognosis (Bullinger et al., supra; Stratowa, C., et al. (2001). CDNA microarray gene expression analysis of B-cell chronic lymphocytic leukemia proposes potential new prognostic markers involved in lymphocyte trafficking. International Journal of Cancer 91, 474-480; Valk, P. J. M., et al. (2004). Prognostically useful gene-expression profiles in acute myeloid leukemia. The New England journal of medicine 350, 1617-1628), and there has been some success in prediction of the response of patients to chemotherapy (Raponi, M., et al. (2007). Identification of Molecular Predictors of Response in a Study of Tipifarnib Treatment in Relapsed and Refractory Acute Myelogenous Leukemia. Clinical Cancer Research 13, 2254-2260; Willenbrock, H., et al. (2004). Prediction of immunophenotype, treatment response, and relapse in childhood acute lymphoblastic leukemia using DNA microarrays. Leukemia 18, 1270-1277). Despite significant evidence for the variability of cell types present in the population of cancer cells most of the AML microarray studies have been based on unsorted cells. However stem cell-based gene expression signatures even as measured in unsorted bulk sample were shown to be an effective tool for outcome prediction (Gentles, A. J., et al. (2010). Association of a leukemic stem cell gene expression signature with clinical outcomes in acute myeloid leukemia. JAMA 304, 2706-2715; Heuser, M., et al. (2005). Gene-expression profiles and their association with drug resistance in adult acute myeloid leukemia. Haematologica 90, 1484-1492). Expressional signature analysis resulted in identification of CD47 (Majeti, R., Chao, M., Alizadeh, A., and Pang, W. (2009). CD47 Is an Adverse Prognostic Factor and Therapeutic Antibody Target on Human Acute myeloid leukemia stem cells. Cell 138(2):286-99; Jaiswal S et al. (2009). CD47 is upregulated on circulating hematopoietic stem cells and leukemia cells to avoid phagocytosis. Cell 138(2):271-85) and TIM3 (Jan, M., et al. (2011). Prospective separation of normal and leukemic stem cells based on differential expression of TIM3, a human acute myeloid leukemia stem cell marker. Proceedings of The National Academy of Sciences USA 108, 5009-5014) as important prognostication markers and players in leukemia.

In addition to variability imposed by clonal origin and lineage hierarchy a significant difference in response of AML blasts to extracellular stimuli could be observed. Abnormal signaling response is a major driving force of cancer and is thought to be associated with mutations that produce hyper-phosphorylated and constitutively activated intermediates of cytokine signaling pathways (Kelly, L. M., and Gilliland, D. G. (2002). Genetics of myeloid leukemias. Annual review of genomics and human genetics 3, 179-198). Screening the AML cells by recently established intracellular flow cytometry (Schulz, K. R., et al. (2012). Single-cell phospho-protein analysis by flow cytometry. Current protocols in immunology/edited by John E Coligan [et al] Chapter 8, Unit8.17) showed discernable differences between the signaling patterns observed in normal and leukemic cells (Han, L., et al. (2009). Single-cell STAT5 signal transduction profiling in normal and leukemic stem and progenitor cell populations reveals highly distinct cytokine responses. PLoS ONE 4, e7989; Irish, J. M., et al. (2004). Single cell profiling of potentiated phospho-protein networks in cancer cells. Cell 118, 217-228; Kotecha, N., et al. (2008). Single-cell profiling identifies aberrant STAT5 activation in myeloid malignancies with specific clinical and biologic correlates. Cancer Cell 14, 335-343) as well as significant heterogeneity in response of leukemic cells to cytokines even within individual patients. Studies from two groups have come up with a strategy to predict the response to chemotherapy based on the pattern of signaling response induced by a panel of diagnostic cytokines (Irish et al., supra; Kornblau, S. M., et al. (2010). Dynamic single-cell network profiles in acute myelogenous leukemia are associated with patient response to standard induction therapy. Clinical Cancer Research 16, 3721-3733; Rosen, D. B., et al. (2010). Distinct patterns of DNA damage response and apoptosis correlate with Jak/Stat and PI3kinase response profiles in human acute myelogenous leukemia. PLoS ONE 5). Among the stimuli used in these studies, IFN and G-CSF were found to be most prognostically significant. In particular, patients with a relatively high number of peripheral blasts that respond to G-CSF were shown to have poor prognosis.

We aimed to understand the nature of the subsets of AML blasts that respond differently to G-CSF and therefore link the map of the signaling response to the map of cancer lineage as proposed by stem cell paradigm. For that matter a unique microarray screening procedure has been established to examine the gene expression in sorted cells stained against intracellular antigens. Samples from a group of AML patients whose blast cells manifested a heterogeneous response to G-CSF were analyzed; about half of AML patients have this type of response to G-CSF. Based on gene expression and signaling data, this group was divided into two subgroups. The leukemic cells from the Type 1 subgroup could partially differentiate, whereas those from the Type 2 subgroup had a strong differentiation block. The signaling threshold separating these two groups was determined, and new prognostic markers such as JAM-A/F11R/CD321 and CD11a applicable for categorization of cell types and patients even in the absence of signaling data were identified. The Type 1 CD321^{high} clonogenic population included primitive cells that responded to G-CSF. This suggests that CD321 could be employed as a tool for their elimination.

### Materials and Methods

Patient Samples and Preparation.

The study was approved by the Stanford Institutional Review Board and samples were collected after informed consent. Samples were collected from a group of patients with AML and high peripheral blood blast counts. These patients were admitted to the Stanford Hospital from April 2000 to August 2010, the median age was 60 with ages ranging from 29 to 84. Enriched AML cell populations containing cancer cells were prepared using a simple density gradient separation of peripheral blood samples (Ficoll-Hypaque, NyCoMed; specific density 1.077) before standard cryopreservation.

Patient samples were thawed by gently adding cells into DMSO medium that contains 2 mM EDTA and 10% FBS, then gently mixed the cells followed by centrifugation at 500 g for minutes. Resuspend the cell pellet in RPMI with 10% FBS and let rest for one hour at 37° C. incubator before applying the cells for live cell sorting, phospho-flow with RNA extraction.

Isolation of Intact RNA from Stimulated, Fixed and Permeabilized Stained and Sorted Cells.

Following treatment with G-CSF (20 ng/ml for 15 min at 37 C) cells were fixed for 10 min at room temperature (RT) by adding 16% formaldehyde (100 μL/mL) directly into tubes with media (RPMI, 10% FCS). A stock of fixed cells was pelleted by centrifugation at 500 g for 5 min and was re-suspended in a residual volume by vortexing. Ice-cold methanol was added, and cells were either kept on ice for 10 min prior to analysis or stored at −80 C. Cells in methanol were spun and resuspended in 1:1 mixture of methanol with 10% formaldehyde in PBS, 0.1% Tween 20 (PBST), kept for 5 min on ice, then spun and resuspended in 10% formaldehyde in PBST and kept for 20 min at RT. Following this post-fixation, cells were washed twice with PBST/RNasin (40 units/ml) and stained with an antibody cocktail in the presence of RNasin (800 units/ml). Following staining, cells were washed twice with PBST/RNasin (40 units/ml) and stored until sorting in PBST/RNasin (800 units/ml). Cells were sorted into PBST/RNasin (80 units/ml) and RNA was isolated using the RecoverAll kit (Ambion). It was crucial to add 11 of RNasin (40 units/up to the 601 DNase digestion mix

Probe Synthesis and Microarray Hybridization.

Affymetrix microarrays were used to determine levels of gene expression in sorted cell subsets from AML samples. Microarray hybridization probes were synthesized from total RNA using an Ovation Pico WTA kit (NuGEN) and were biotin labeled using the Encore Biotin Module (NuGEN). The probes were hybridized to Human Genome U133 Plus arrays. The Agilent Human Whole Genome Oligo Microarray was used to analyze unsorted Flt3-positive samples from the 2004 cohort (Irish et al., supra) RNA was isolated from AML blasts and purification of poly(A) RNA was performed. Aminoallyl-U (aa-UTP; Ambion) was incorporated into cRNA followed by cross-coupling of Cy5 and Cy3 by means of reactive Cy-NHS compounds (Amersham Biosciences) in order to generate fluorochrome-labeled targets for DNA microarray analysis. Stratagene Universal RNA was used for reference for the Cy3-aa-cRNA preparation (Stratagene). The hybridization procedure was performed according to the Agilent protocols, except that a more stringent wash step was incorporated (0.1×SSC at 35° C. for 10 min).

Antibodies Used for Flow Cytometry and Cell Sorting.

The following antibodies were purchased from BD Biosciences: purified antibodies for phospho-STAT3 (pY705), phospho-STAT5 (pY694), CD11a, CD36, QDot605 labeled anti-CD45, Pacific Blue labeled anti-CD3, FITC labeled anti-CD34, PerCP-Cy5.5 labeled anti-CD33. Purified CD321 were purchased from eBiosciences. Conjugation of NHS-Ax488 and NHS-Ax647 dyes (Invitrogen) to purified antibodies was done according to protocols supplied by manufacturer. For sorting the blast cells the total population of fixed, permeabilized, stained PBMC was gated as SSCmedium-high, CD45-medium, CD3-negative. Signaling subsets were sorted from gated blast cells according to phospho-Stat5 phospho-Stat3 staining. CD3-positive T cells were sorted for RNA quality control.

Colony Formation Assay.

Colony formation assays were done in MethoCult H4434, (StemCell Technologies). Following manufacturer's instruction, cells were plated into the culture medium immediately after live cell sorting. Colonies were counted under a reverse phase microscope Nikon TMS at the fourteenth day after culture in a chamber with water in 37 C incubator.

Secreted Cytokine Analysis.

Sorted AML blast cells with differential surface protein expression were cultured in IMDM with 30% FBS, with 100 units of penicillin and 100 ug of streptomycin per ml for 24 hours. After that, the cultured blast cells were pelleted by centrifugation at 500 g for 10 minutes. Supernatants were collected, stored in −20 C before being analyzed by Luminex human 51-plex cytokine panel. Luminex assay were performed by Human Immune Monitoring Laboratory in Stanford.

Data Analysis.

The raw data from hybridizations performed on Affymetrix microarrays (both for the datasets created in this study as well as for publicly available data) was subjected to quantile normalization using RMA-express software based on remapped custom ENSENG .cdf files (hgu133plus2hsensgcdf version 13.0.0; http://brainarray.mbni.med.umich.edu/Brainarray). The two-channel Agilent oligonucleotide microarrays were scanned (Agilent Scanner G2505B) and features automatically extracted using Agilent Feature Extraction v.7.5.

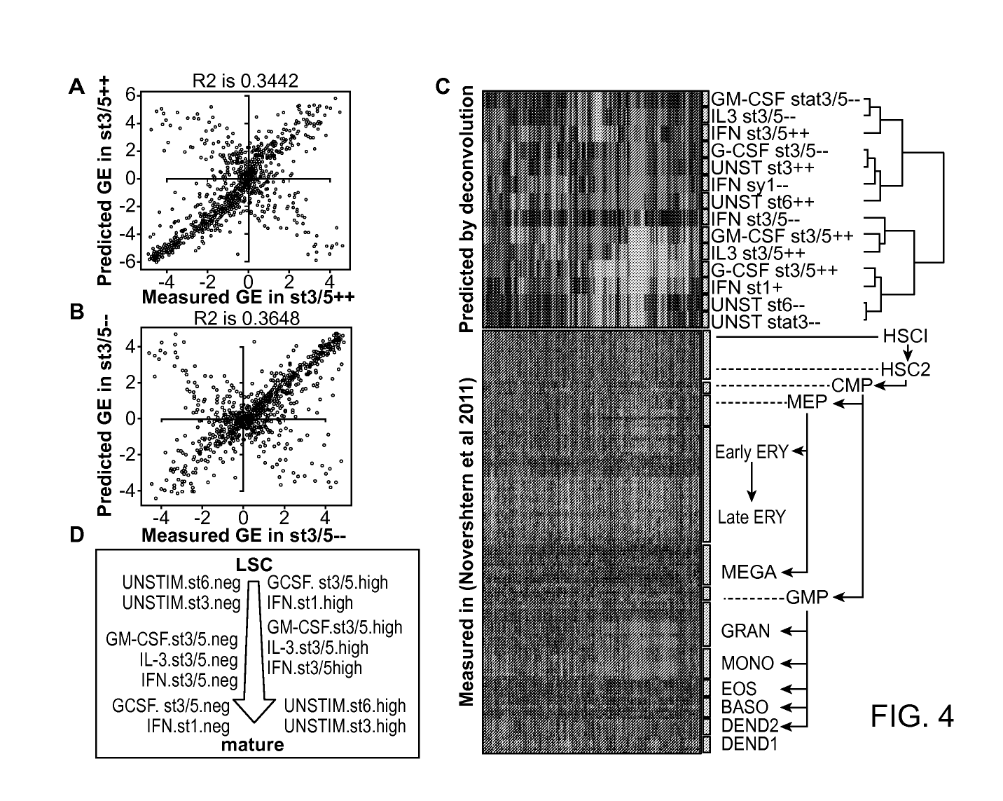

Deconvolution by linear modeling was performed in R. Briefly the two-channel Lowess-normalized Agilent LogRatio data from Flt3-positive patients of the 2004 cohort was filtered by removing the genes with more than five NAs (rlsWellAboveBG=0, glsWellAboveBG=0) out of 14 measurements, was centered by subtracting the means, and was transformed into natural numerical space. Linear model assuming a mixed composition of the unsorted data using percentages of specific subsets calculated in 2004 study was fit using the function(x){lm(x˜0+statNEGATIVE+statPOSITIVE)$coefficients}). The derived $coefficients represented the average gene expression in statNEGATIVE or statPOSITIVE subsets. For cross dataset comparisons of predicted versus experimentally derived data (FIG. 4A), expression values derived from Type 1 cells were transformed into natural numerical space, z-normalized by subtracting the mean and dividing by standard deviation, and then multiplied by the standard deviation of centered data from 2004 study. Due to the wide range, asinh transformed values were used for cross data comparisons on biaxial plots in as well as for hierarchical clustering.

For Spade clustering, all stimulated bone marrow and peripheral blood mononuclear cell (PBMC) flow cytometry datasets were singlet-gated, live cell gated (by removing cells that were positive for cleaved PARP), compensated in FlowJo, exported as FCS files, and imported into SPADE for analysis as recently described (Qiu, P., et al. (2011). Extracting a cellular hierarchy from high-dimensional cytometry data with SPADE. Nature Biotechnology 29, 886-891). SPADE analysis was performed using an arcsinh cofactor of 150 and 120 target clusters. All conditions from a single experiment were processed simultaneously so that the resulting tree structure would capture all cell surface subpopulations present in the entire dataset.

### Results

Isolation of Intact RNA from Cells Post Fixation and Permeabilization.

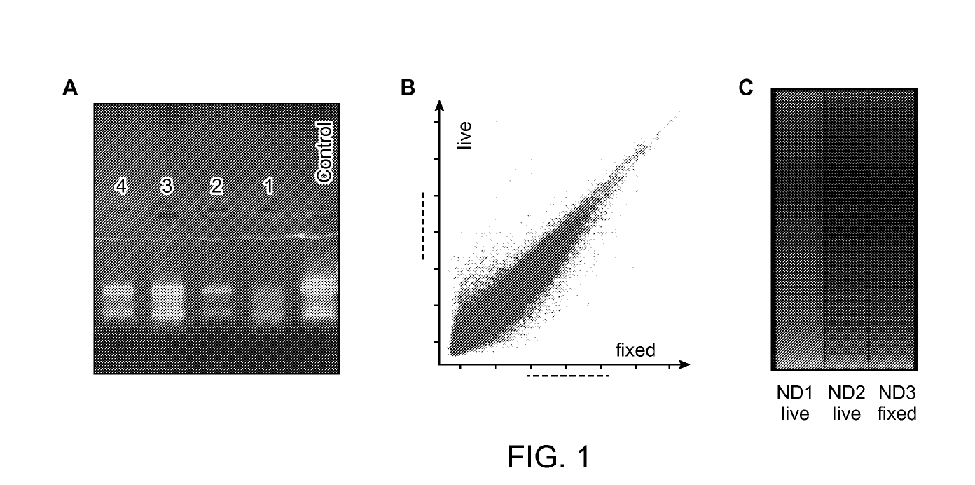

In order to measure gene expression in cell subsets defined by their intracellular signaling responsiveness, a methodology for isolating intact RNA from fixed and permeabilized cells was established. The most critical step to maintain RNA integrity was a second paraformaldehyde fixation post, the routinely performed, fixation and methanol permeabilization steps applied to sample processing for multiparameter cytometry (data not shown). This additional fixation step was found to be essential for complete inactivation of RNases released during sample processing. Gel electrophoretic analysis of RNA isolated from untreated (Control) U931 cells or from U931 cells treated with the fix-permeabilize-fix protocol indicated that RNA integrity was well preserved (FIG. 1A). To verify that these conditions left RNA intact in primary samples, live monocytes (CD33+) and T cells (CD3+) were sorted from PBMCs from three healthy donors (ND1, ND2, ND3). RNA was directly prepared from the sorted monocytes and T cells from ND1 and ND2. The sorted monocytes and T cells from ND3 were subjected to the fix-permeabilize-fix protocol, followed by incubation with antibodies that were to be used later in screening of the leukemic blasts (anti p-Stat3 and p-Stat5) and re-sorted to mimic the “sorting after staining” scenario. Whole genome expression profiles were compared from live and fixed cells using the Affymetrix U133 Plus 2 microarray and were highly correlated (FIG. 1B). Furthermore, the heatmaps show good accord between live versus fixed cells for the logRatio of gene expression in monocytes versus T cells (FIG. 1C). The data therefore support using this new methodology for isolating high quality RNA from fixed cells.

mRNA Expression Signatures Reveal Two Different Stages of Hematopoietic Maturity Between AML Blast Subsets.

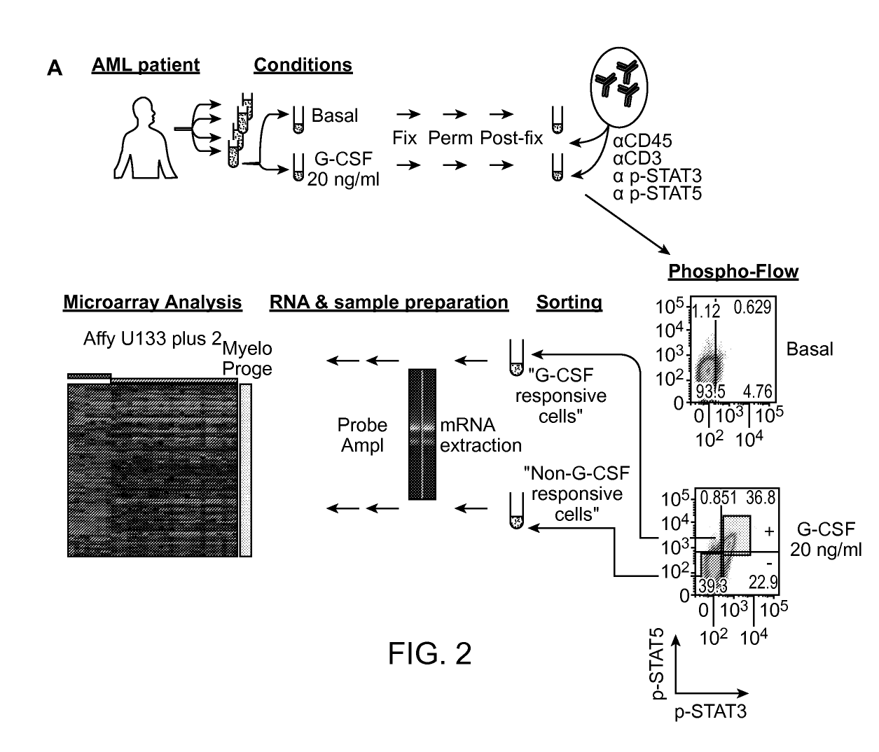

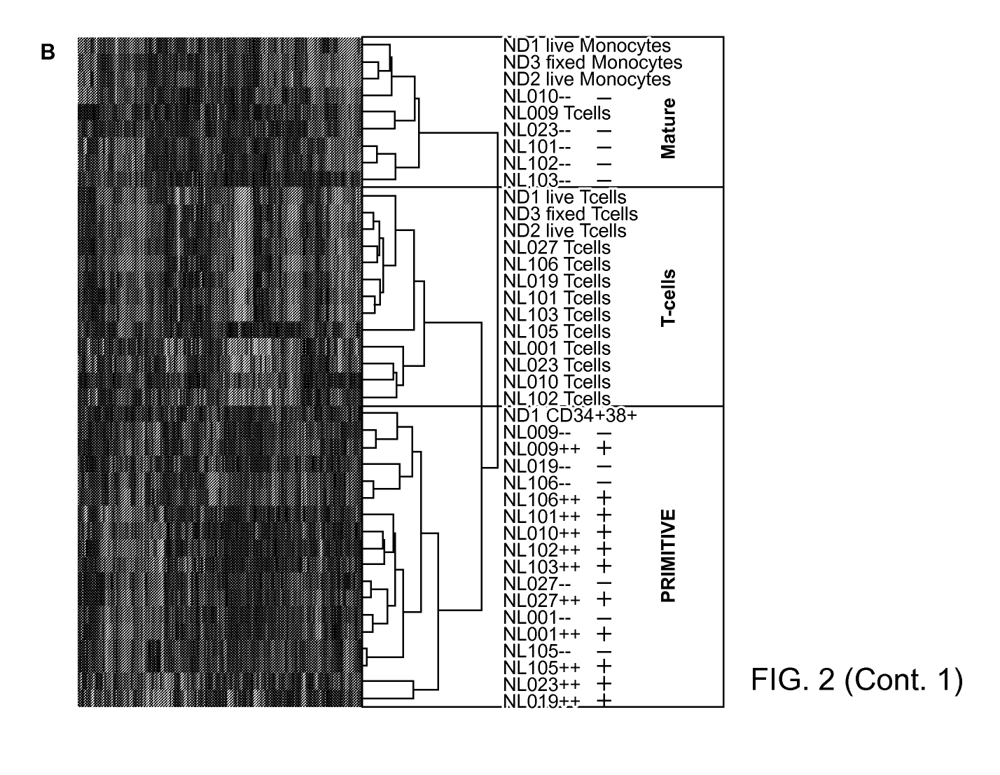

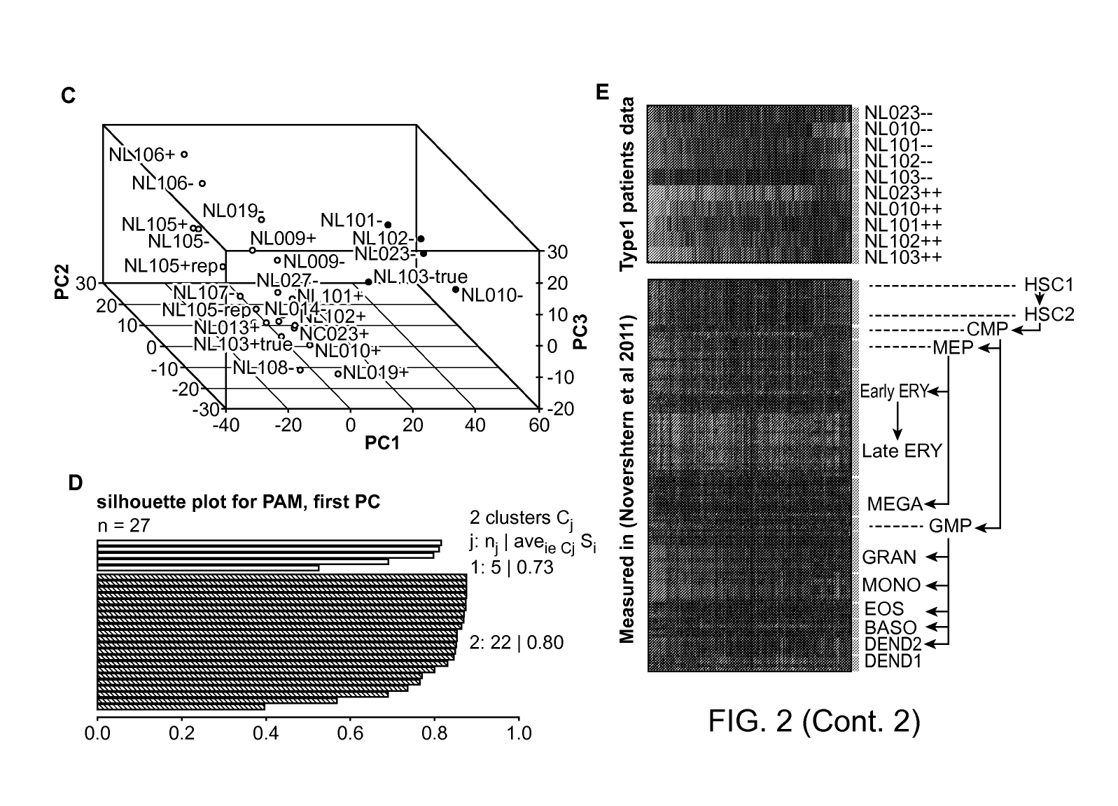

To analyze the gene expression in AML blast subsets with distinct signaling responses, cryopreserved PBMC samples from AML patients were pre-screened using p-STAT3 and p-STAT5 as intracellular readouts to identify those samples where a pronounced G-CSF responsive cell subset (as shown on the workflow diagram on FIG. 2A) could be observed. Eleven AML samples in which greater than 0.5% of cells were responsive to G-CSF treatment (Table 1) were selected for further detailed molecular analysis.

TABLE 1
The clinical information for 11 AML patients
whose cells were evaluated in this study^{a}

| | | | Blast % | | FAB categories, |
|---|---|---|---|---|---|
| Patient | | | (CD34% in | AML diagnosis | cytogenetic or |
| ID | Sex | Age | blasts)^{b} | status | genetic mutation^{c} |
| NL001 | M | 58 | 60% (28%) | de novo AML | M2 |
| NL009 | F | 51 | 31% (94%) | Transformed | NK |
| | | | | from myeloid | |
| | | | | fibrosis | |
| NL010 | F | 45 | 99% (≦1%) | de novo AML | M4, NPM⁺ AML |
| | | | | | Flt3⁺, |
| NL019 | F | 50 | 29% (n.d.) | Transformed | NK |
| | | | | from MDS | |
| NL023 | M | 76 | 29% (86%) | Transformed | NK |
| | | | | from MDS | |
| NL027 | F | 18 | 71% (90%) | de novo AML | M5a Trisomy 8, |
| | | | | | Flit3⁺, CEBPα⁺ |
| NL101 | F | 51 | 95% (≦1%) | de novo AML | M4 |
| NL102 | M | 49 | 96% (≦1%) | de novo AML | M4, |
| NL103 | M | 47 | 86% (27%) | de novo AML | M5b |
| NL105 | M | 59 | 82% (14%) | Relapse | M5b |
| NL106 | F | 66 | 74% (n.d.) | Relapse | N/A |

^{a}This cohort contains a mixture of patients with different blast population sizes, CD34 representations, French-American-British classifications, cytogenetic and genetic mutations and primary vs. secondary AML.
^{b}n.d. indicates not determined for this sample.
^{c}NK indicates normal karyotype without identified mutations.

Larger aliquots of these samples were treated with G-CSF, subjected to the new fixation/permeabilization protocol, incubated with antibodies against CD45, CD3, p-STAT3 and p-STAT5 and sorted to separate the AML blast (CD45^{mid}) cells with increased G-CSF-mediated p-STAT3 and p-STAT5 levels (referred to as G-CSF responsive cells, or “GRCs”) and those non-responsive to G-CSF (“NGRCs”). To monitor the per-patient quality of the RNA isolation, T cells (as defined by CD3+ staining) were sorted simultaneously with signaling subsets. Fresh and fixed T cells and monocyte from ND1-3 (FIG. 1B,C) were used as a reference for high quality RNA. Additionally, CD34+CD38+ progenitor cells were sorted from healthy ND1 PBMCs. Following RNA isolation and probe preparation by isothermal amplification for each cell subset, their gene expression signature was determined by microarray hybridization and the data was visualized using heatmaps (FIG. 2A).

Hierarchical co-clustering of the microarray data from the AML cell subsets (GRCs and NGRCs) with microarray data from progenitor, myeloid and T cells from healthy PBMCs (described above) revealed three distinct clusters (FIG. 2B). One cluster segregated genes from T cells in AML samples with T cells from healthy donors (“T cells” FIG. 2B). For one sample, (NL009), the T cell mRNA genes co-clustered with healthy monocytes despite high levels of expression of T cell markers such as CD3g. The remaining two clustered were represented by myeloid cells from normal donors and sorted signaling subsets of the AML blasts. In particular, the second major cluster was comprised of expression profiles from NGRCs from 5 AML samples and expression profiles of monocytes from healthy PBMCs. These data suggest a more differentiated phenotype for these NGRCs. In the third cluster mRNA expression profiles from NGRCs and GRCs from AML samples segregated with CD34+CD38+ progenitor cells from healthy PBMCs, suggesting that these AML cell subsets bore resemblance to a hematopoietic progenitor phenotype. GRCs from all AML samples tested were of this phenotype, whereas the NGRCs from only a subset of AML samples were in this cluster. That there were two classes of gene expression from AML patients was further supported by principal component analysis (FIG. 2C) and partitioning around medioids (PAM) silhouette width profiling (FIG. 2D).

Hematopoietic Maturity and Ratio of GRCs and NGRCs within a Sample Determines AML Type.

By comparing mRNA expression signatures from AML GRCs and

NGRCs with signatures of different hematopoietic maturity from healthy PBMCs, the AML samples from this eleven-sample cohort could be segregated into two types. In Type 1, (NL101, -102, -103, -010 and -023) NGRCs and GRCs resemble more differentiated and progenitor-like signatures respectively. In Type 2 (the remainder of the cohort, i.e. NL009, -19, -106, -027, -001 and -005) both GRCs and NGRCs resemble a progenitor-like signature. Importantly, expression of the top 700 differentially expressed genes from Type 1 samples was compared with the publically available data on gene expression in hematopoietic lineages (Novershtern et al., supra). Genes expressed at higher levels in p-STAT5-positive cells of Type 1 samples were generally expressed in stem cells and progenitors, whereas genes expressed at higher levels in cells of Type 1 samples that did not respond to induction by G-CSF were primarily expressed in monocytes (FIG. 2E) and other mature cells of myeloid lineage.

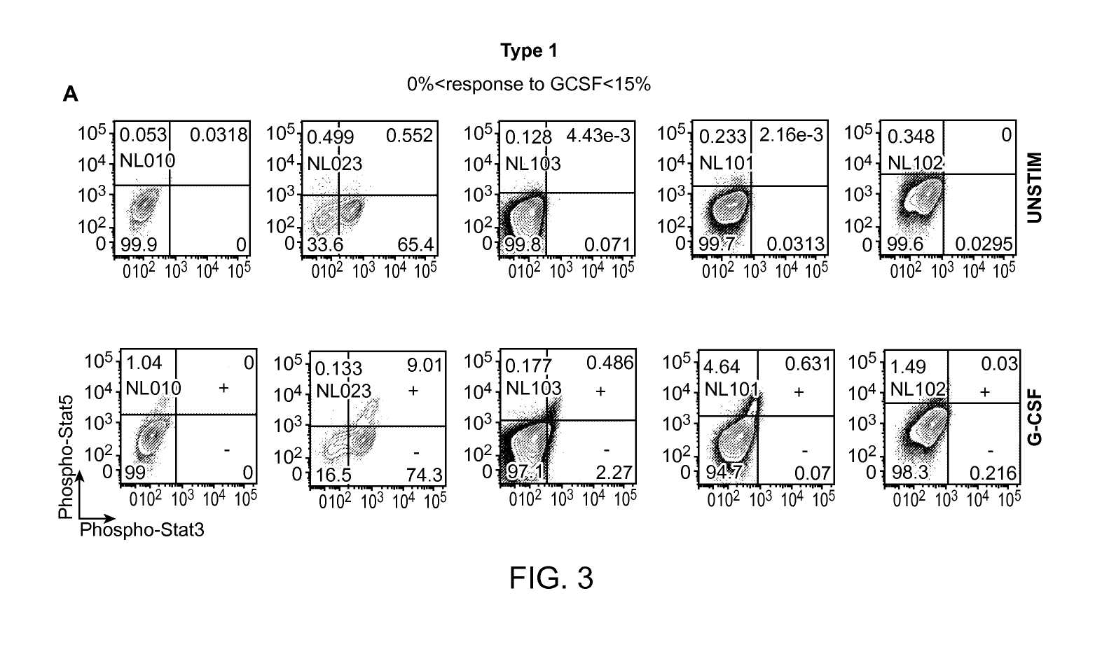

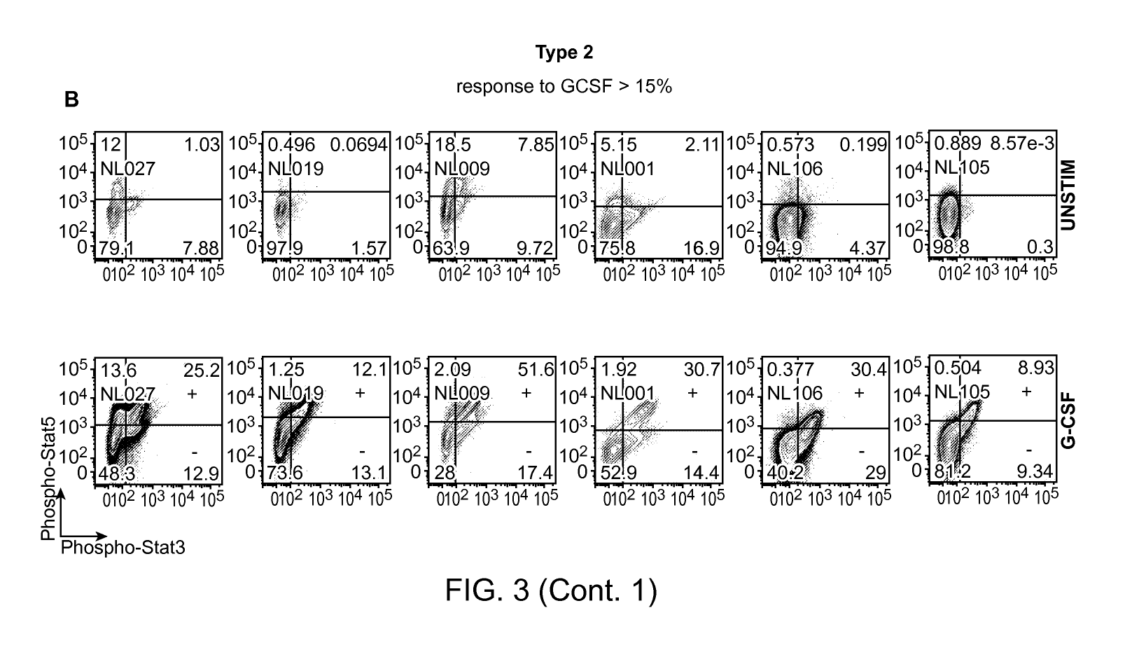

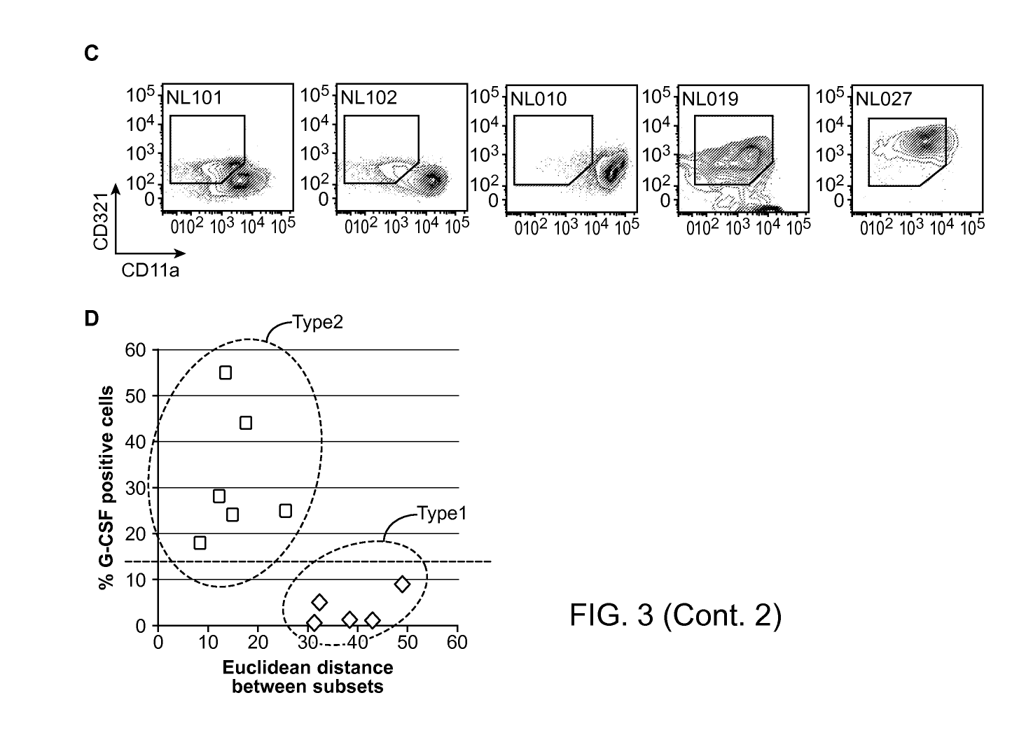

To see how signaling response is linked to gene expression, the correspondence between the response to G-CSF in AML blasts and the difference between the signaling subsets as defined by gene expression was examined. The percentage of GRC was measured by flow cytometry in Type 1 and Type 2 samples (FIG. 3A, B), and plotted against the Euclidean distance between the GRC and NGRC in each individual patient, calculated based on 700 genes differentially expressed between healthy mature monocytes and myeloid progenitors (FIG. 3 D). Two distinct clusters of patients could be observed. In average, Type 1 patients shared high distance between the subsets and low ratio of GRC whereas Type 2 patients had high numbers of GRC and low distance between the subsets. Since both signaling subsets of Type 2 patients were undifferentiated and the majority of cells (as represented by NGRC) in Type 1 patients were relatively mature it would be reasonable to assume that when analyzed in bulk, cell of Type 1 patients manifest overall mature and Type 2 immature gene expression patterns.

The separation of patients into two groups as defined by gene expression and signaling points to existence of a dedicated biological mechanism underpinning the early differentiation block in Type 2 patients. Signaling threshold was calculated as an average between the amount of G-CSF responding cells in Type 1 patient with the highest (NL023-9%) and the Type 2 patient with the lowest G-CSF responding cells (NL105-18%). It was found that threshold of 13.5% (FIG. 3D, red line) separated AML patients with predominantly mature blasts (Type 1, fewer than 13.5% of cells respond to G-CSF with Stat phosphorylation) from Type 2 patients where mature blasts could not be observed.

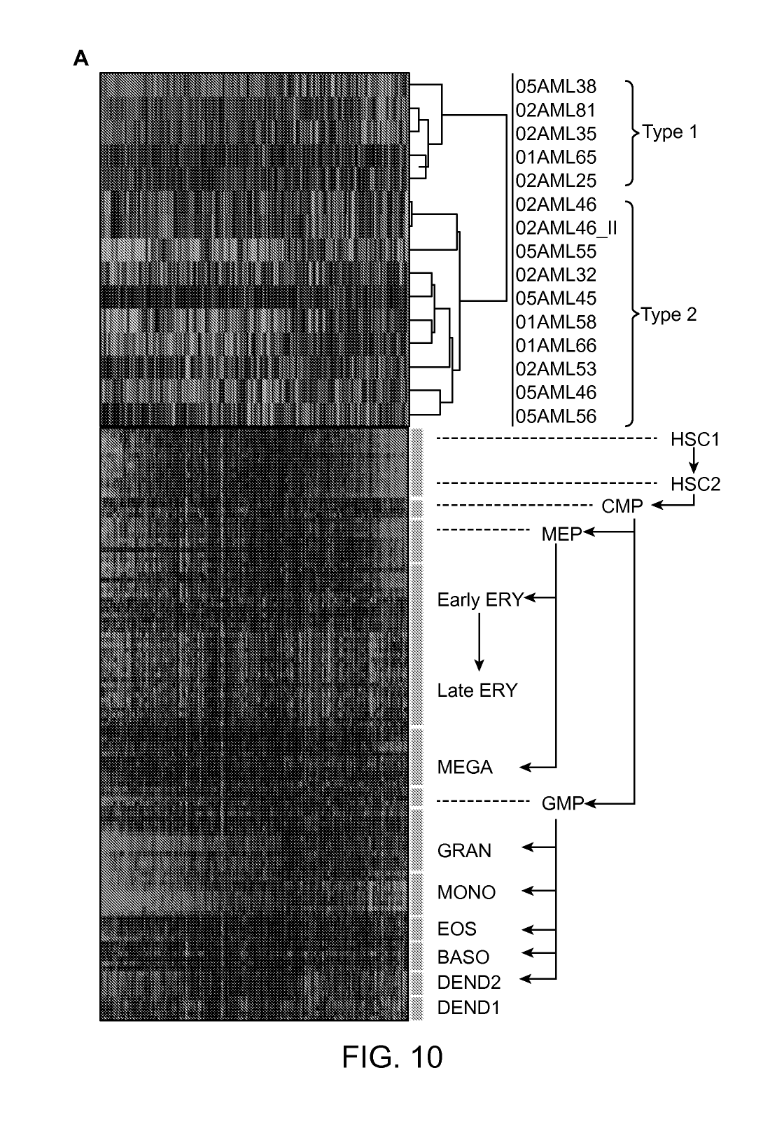

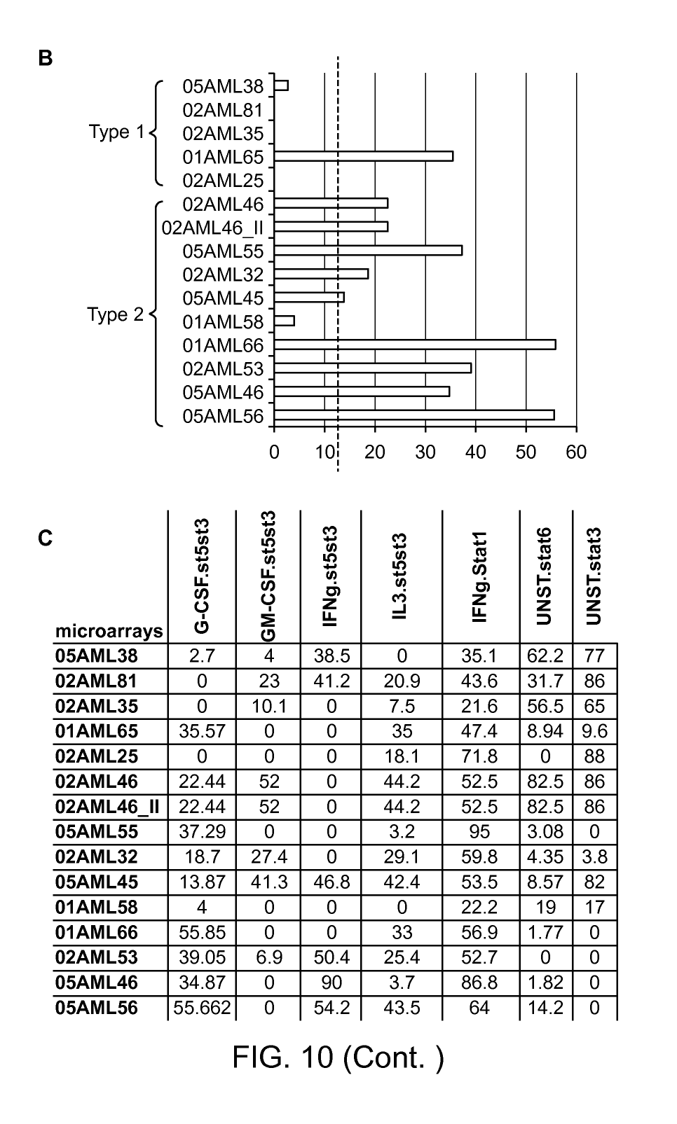

In order to corroborate the 13.5% threshold value, an unpublished microarray data from 15 unsorted AML samples representing a Flt3 mutation positive subset of a cohort analyzed in Irish et al., supra was further examined. These samples are further referred as “the 2004 cohort”. 800 genes were selected based on the highest standard deviation across this dataset and were further filtered to select 500 genes showing a high difference in gene expression between the primitive (HSC and CMP) versus the mature (monocyte and dendritic) cells of the “Broad Institute” dataset (Novershtern et al., supra). These 500 genes were used to hierarchically cluster the 2004 Cohort. Samples segregated into two clusters. The first cluster, containing 5/15 samples, had elevated expression of genes specific to the mature myeloid lineage and therefore resembled Type 1 AML, where the majority of blasts showed relatively mature gene expression phenotype. In the second cluster, 10/15 samples expressed genes specific to progenitor cells and hence could be classified as Type 2 (FIG. 10A). Out of five samples of the Type 1-like cluster, four samples had less then 13.5% cells responding to G-CSF, and eight out of nine Type 2-like samples had more than 13.5% GRC as measured in Irish et al., supra. Thus, the 13.5% signaling threshold robustly separates patients with Type 1 versus Type 2 AML.

Deconvolution Analysis Correctly Predicts Average Gene Expression Profile of GRCs.

The importance of hematopoietic cytokines in sustaining or in some cases inhibiting the cancer lineage has been underscored by prior studies. To pinpoint the origin of cells responding to cytokines, the deconvolution analysis (Shen-Orr, S. S., et al. (2010). Cell type-specific gene expression differences in complex tissues. Nature Methods 7, 287-289) based on percentages of responding cells as measured in Irish et al., supra was applied to the microarray data from unsorted cells of the 2004 cohort. Although the present and the 2004 AML cohorts were analyzed by two different microarray platforms (Affymetrix U133 Plus 2 one-color assay for sorted signaling subsets versus Agilent Human Genome two-color for the 2004 cohort), a good linear correlation was found between the measured levels of gene expression in GRCs and NGRCs in Type 1 AML with the corresponding computational predictions (FIG. 4 A, B). In particular, matching the genes predicted to be differentially expressed between GRCs and NGRCs against publically available data (Novershtern et al., supra) revealed that genes specific to mature blood cells had higher levels in predicted NGRC gene expression data (FIG. 4C). The distinguishing attribute of Type 2 AML as defined in this study was lack of pronounced gene expression difference and a common progenitor signature observed both in GRC and NGRC. Therefore the de-convolution based prediction was inaccurate for Type 2 patients. Also judged by the percentage of G-CSF responding cells (FIG. 10C) in 2004 cohort less then half of the AML patients belonged to Type 1 and thus the bias of prediction towards Type 1 patient could not be explained by the absence of Type 2 patients from the 2004 cohort. Therefore if applied to an unbiased cohort where Type 1 and Type 2 patients are equally represented, rather then delivering the prediction that would be correct in each individual patient, the deconvolution analysis seems to have properly captured the trend—whereby cells responding to G-CSF are less mature and the cells not responding to G-CSF are in more mature stages of differentiation.

This computational strategy was then applied to derive the average gene expression profiles within cellular subsets responding to other cytokines as described in the 2004 Cohort study (Irish et al., supra). On average, cells with G-CSF-mediated phosphorylation of Stat3 and Stat5 or IFNγ-mediated Stat1 phosphorylation or low levels of basal Stat6 and Stat3 phosphorylation had a “progenitor”-type gene expression profile. Cells non-responsive to G-CSF or IFNγ had mRNA expression profiles that resembled mature myeloid cells. Cells with GM-CSF, IL3, and IFNγ-mediated Stat3 and Stat5 phosphorylation occupied intermediate positions on the hematopoietic lineage tree and were more primitive than cells that did not responding to these stimuli, although the last are more primitive then cells not responding to G-CSF (FIG. 4C, D).

GRC of Type1 AML Samples are Enriched within CD321 Positive Clonigenic Compartment of Type 1 Samples.

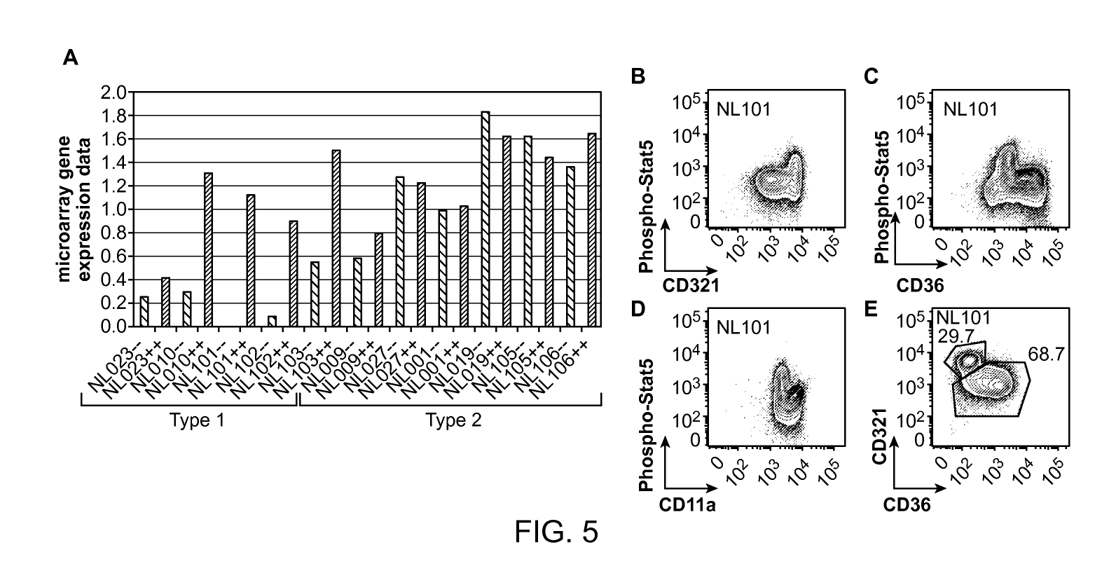

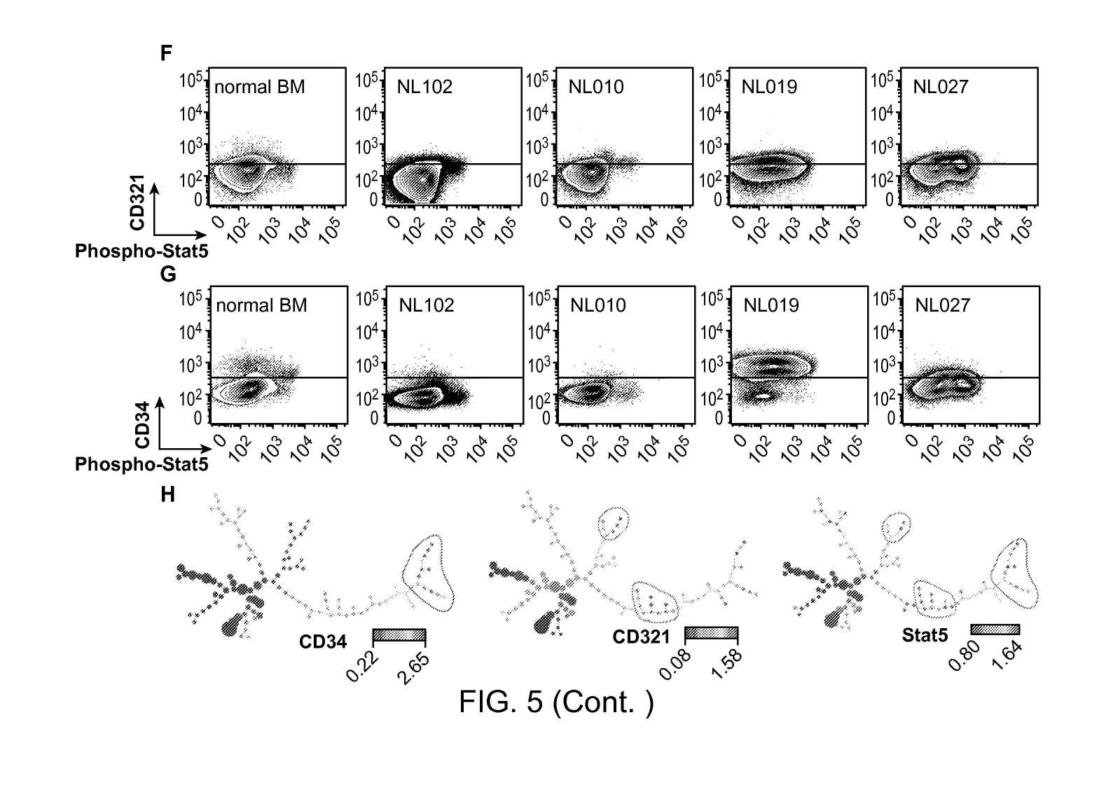

One purpose of the mRNA studies was to identify surface markers that would be expressed with mutual exclusivity in GRCs and NGRCs in order to enable purification of viable cell subsets for further characterization. To that end, microarray data revealed that Type 1 AML samples GRCs expressed elevated levels of the CD321 gene while NGRCs expressed elevated levels of CD36, CD1, and CD11a, which are known myeloid markers (FIG. 5A). Flow cytometry mirrored the gene expression at the protein level. In Type 1 AML, GRCs were enriched within the CD321^{high} fraction, whereas NGRCs were enriched within the CD36 and CD11a positive subset (FIG. 5B-E). In Type 2 AML, CD321 was much less effective in separation of signaling subsets mainly due to high levels of expression both in GRCs and NGRCs (FIG. 5A, F). Unlike CD321^{high} expression, CD34 expression did not correlate with phosphorylation of Stat5 induced by G-CSF (FIG. 5A, F-H). Interestingly even without G-CSF stimulation, the pattern of CD321/CD11a distribution on live cells reliably discriminated between the Type 1 and Type 2 AML patients (FIG. 3C).

From the results above, CD321 and CD36 were identified as surface markers against which antibodies could be used to prospectively sort enriched GRCs and NGRCs gated as shown in FIG. 5E for functional assays. For two exemplary Type 1 AML samples (NL101 and NL010), subsets that were CD321^{hi} produced more colonies in methyl-cellulose than the CD36^{hi}/CD32^{lo} cells (Table 2A). Since all cells in Type 2 AML were CD321^{hi} regardless of their response to G-CSF, the same gating strategy isolated most of the cells in these samples. These cells produced on average more colonies then CD321^{high} cells from Type 1 patients (Table 2A, 2B). In conclusion, the higher clonogenic activity of CD321 positive cells concurs with the undifferentiated status of GRC in Type 1 and both GRC and NGRC in Type 2 AML as revealed by gene expression analysis.

Table 2. (A) CD321^{high} positive subsets from of Type 1 patient samples contain clonogenic cells. Colonies were detected in the AML cells from both patient NL101 and patient NL010 that expressed CD321. AML patient samples were sorted into two populations based on the surface level of CD321 and CD36 expression. The sorted cells were plated at a concentration of 25,000 cells/ml for NL010 and 100,000 cells for NL101. No colonies were formed from CD321^{low} cells. (B) Cells of Type 2 patients are more clonogenic then cells of Type 1 patients. Cells of type 2 patients were uniformly CD321^{high}. Yet for comparison with Type 1 patients cells from Type 2 patients were sorted same as for Type 1 patients and population from CD321^{high} plated at a concentration of 25,000 cells/ml. Colonies were detected in the AML cells from Type 2 patients.

TABLE 2A

| Patient ID | CD321^{high} | CD321^{low} |
|---|---|---|
| NL101* | 12 | 0 |
| NL010 | 60 | 0 |

TABLE 2B

| Patient ID | CD321^{high} |
|---|---|
| NL009 | 12 |
| NL010 | 60 |
| NL019 | 84 |
| NL027 | 128 |

Cross-Communication Between the GRC and NGRC of Type 1 AML.

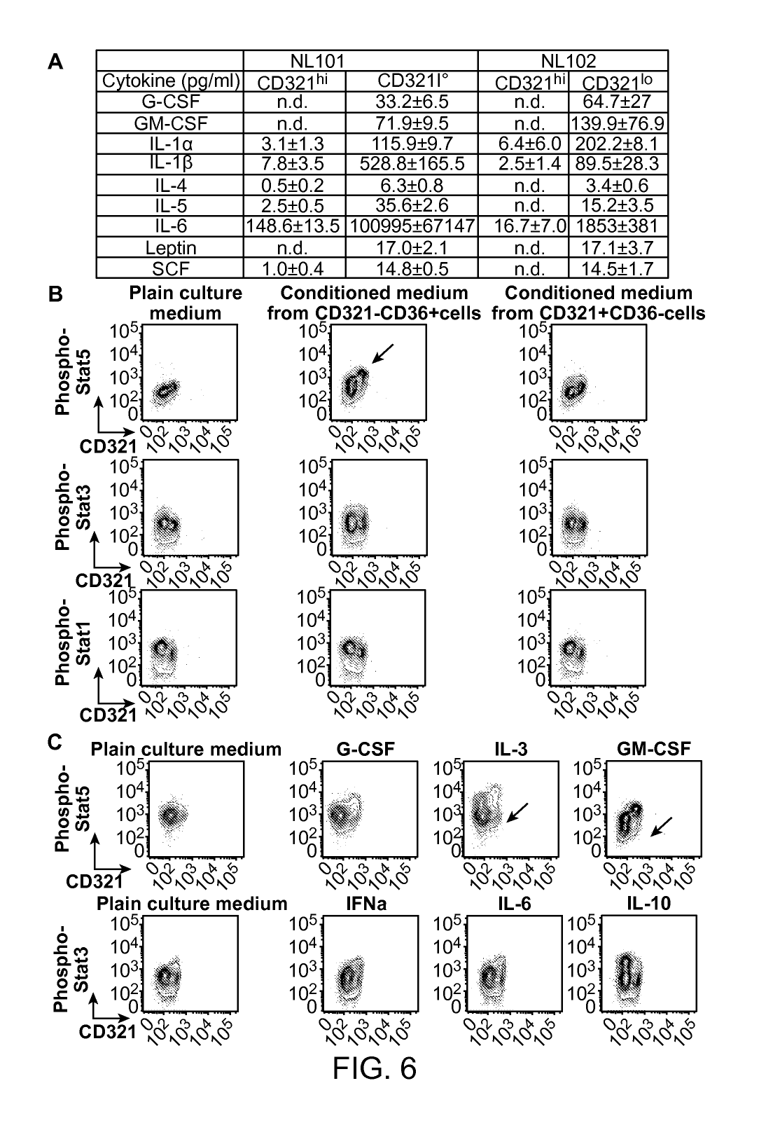

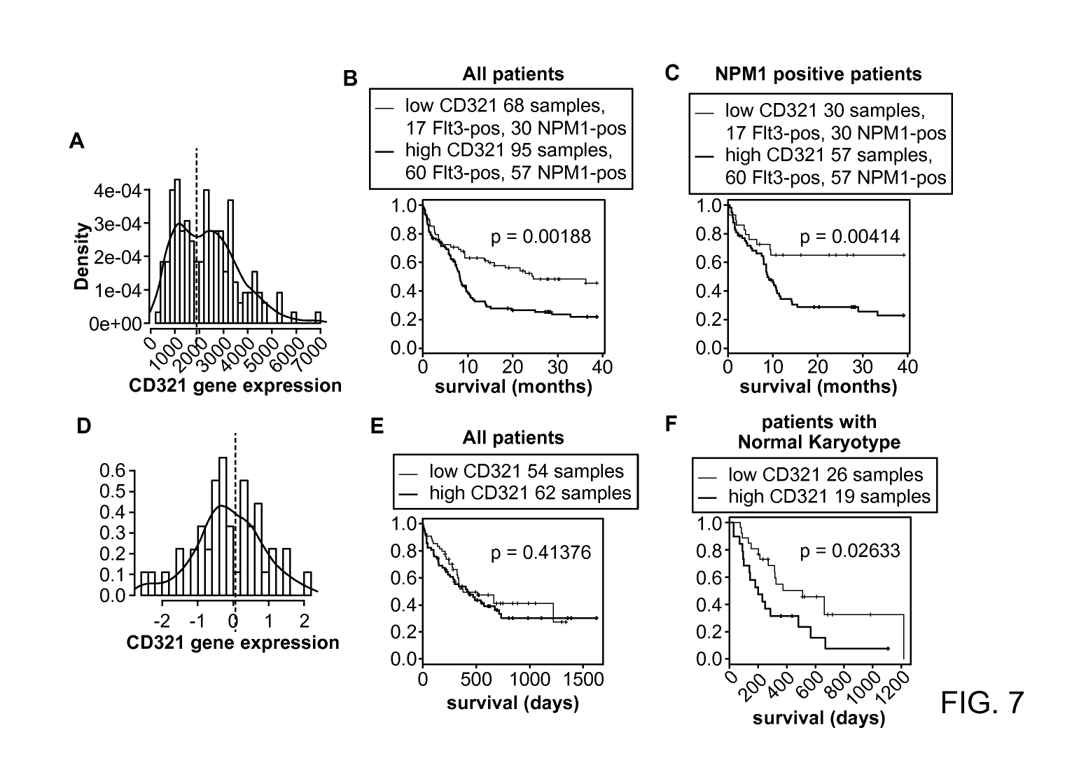

In order to ascertain whether there was a dependence of GRCs and NGRCs toward each other, conditioned medium was generated from sorted Type 1 CD321^{hi}CD36^{lo} and CD321^{lo}CD36^{hi} cells kept in culture for 24 h. When applied to freshly thawed Type 1 AML cells of NL101 only the conditioned medium from CD321^{lo}CD36^{hi} but not from CD321^{hi}CD36^{lo} cell induces significant Stat5 and moderate Stat3 phosphorylation above basal levels (FIG. 6B). Notably this response was localized to the population of CD321^{high} cells hinting at possible cross-communication between the signaling subsets.

In order to identify the active component of the CD321^{low}/CD36^{high} supernatant, a Luminex Human 51-plex bead assay was performed on supernatants produced by sorted GRCS and NGRCs samples NL101 and NL102. The data revealed that levels of several hematopoietic factors were higher in the medium from CD321loCD36hi cells than in the medium from CD321 hiCD36lo cells (FIG. 6A). The effect of individual cytokines selected based on Luminex assays was examined in three Type1 AML samples (NL101,102,010—FIG. 6C and not shown). Despite disproportionally high readings of Luminex for some of the cytokines such as IL-6, GM-CSF—which was detected at modest levels in the conditioned medium produced by CD321^{low}CD36^{high} cells—produced the highest level of response in freshly thawed AML blasts of NL101. Signaling response induced by recombinant GM-CSF (FIG. 6C) was similar to the pattern induced by the conditioned medium (FIG. 6B red arrow). In addition stronger IL-3 response was observed in the population of CD321^{high} cells (FIG. 6B red arrow). This observation is intriguing in view of recent studies indicating that that CD123 interleukin-3 receptor alpha chain is a unique marker for human AML stem cells (Jordan, C. T., et al. (2000). The interleukin-3 receptor alpha chain is a unique marker for human acute myelogenous leukemia stem cells. Leukemia 14, 1777-1784). Due to high numbers of sorted cells necessary for preparing the cultured medium for Luminex assays and limited availability of patient material these experiments were performed only for a limited number of samples.

Prognostic Significance of CD321 Expression on AML Blasts.

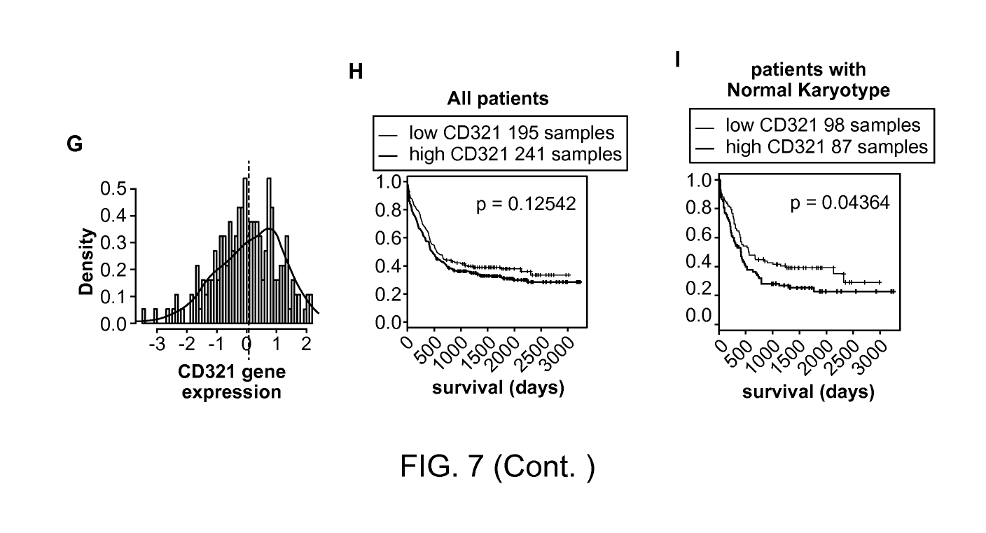

It has been reported that abundance of circulating progenitors as well as the presence of stem cell gene expression signature in microarray data from bulk AML PBMC negatively correlates with prognosis (Eppert, K., et al. (2011). Stem cell gene expression programs influence clinical outcome in human leukemia. Nature Medicine 17, 1086-1093). Considering that primitive Type 1 GRCs reside within the CD321 positive cellular compartment and in view of distinct clonogenic activity observed in CD321^{hi} cells of both AML types, the prognostic significance of CD321 was examined. Bimodal distribution of CD321 levels was observed (FIG. 7 A,D,G and data not shown) in several published AML gene expression datasets indicating the existence of two types of samples. 163 AML patients from the test cohort described in Metzeler et al., supra were split into two groups according to the level of CD321 gene expression (FIG. 7A). The threshold level of CD321 expression was chosen based on the minimum separating the two peaks of the probability density distribution. Survival analysis (FIG. 7B) revealed that patients with overall high CD321 (above threshold) have a lower overall survival rate then patients with lower (below threshold) CD321. The prognostic difference between the groups defined by the levels of CD321 was especially pronounced on the background of NPM1 positive AMLs (FIG. 7C). Notably, choosing the threshold CD321 value based on the average value observed across all patients rather then based on bimodal shape of probability density distribution resulted in noticeably worse separation of survival curves (data not shown). Separation of patients according to CD321 levels partially correlated with the presence of Flt3 mutations. Thus, 63% patients with high CD321 and 25% of patients with low CD321 carried Flt3. Yet even within Flt3-positive or -negative cohorts the CD321 levels still exhibited prognostic potential (data not shown).

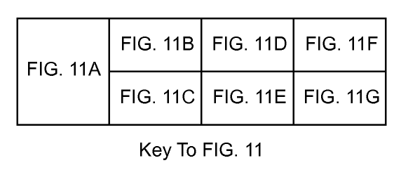

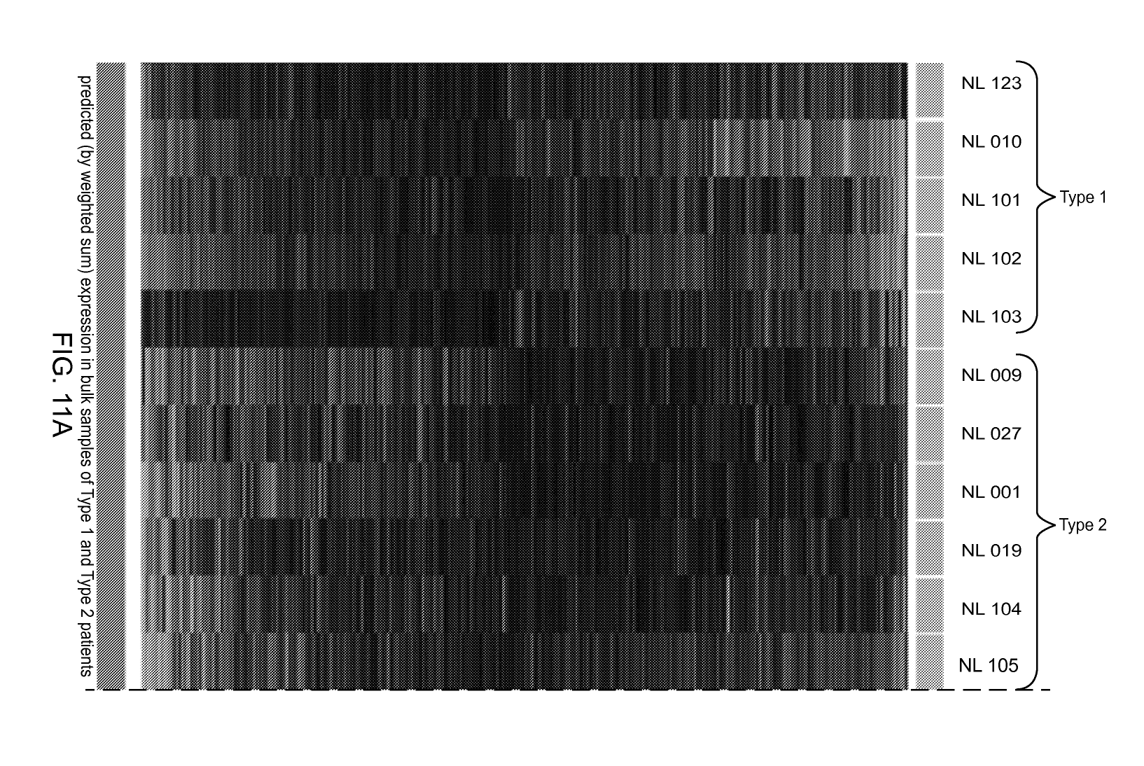

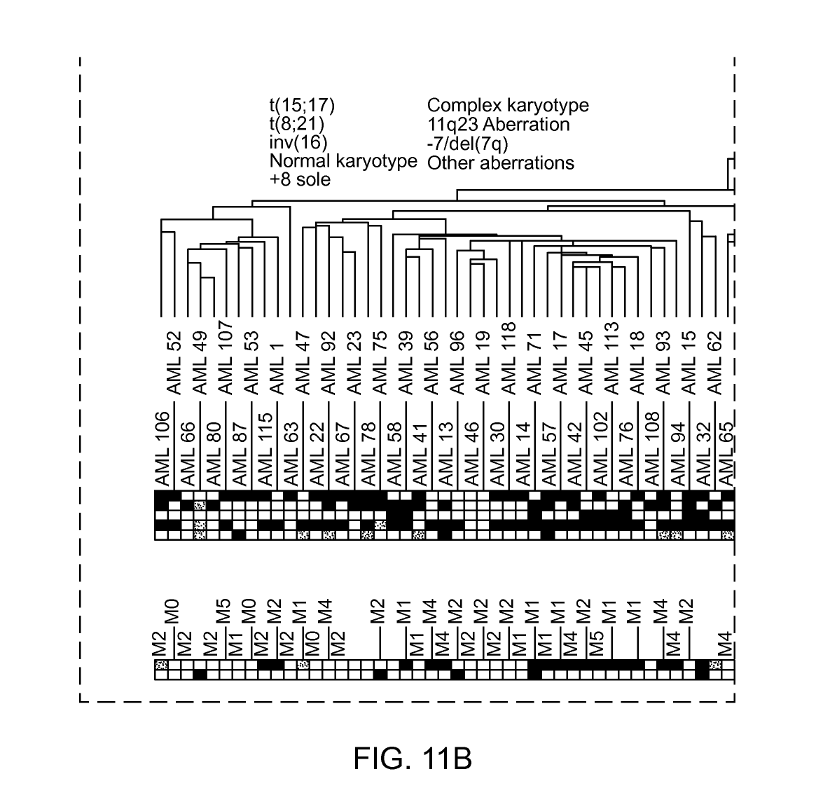

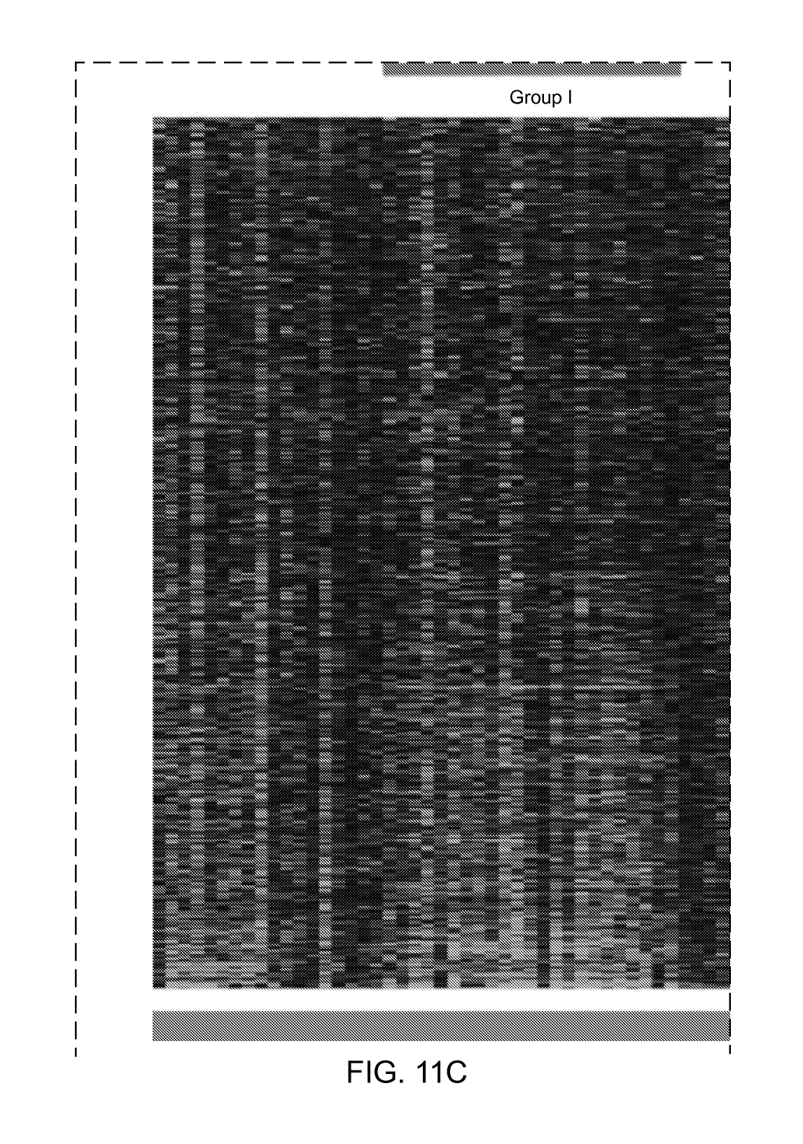

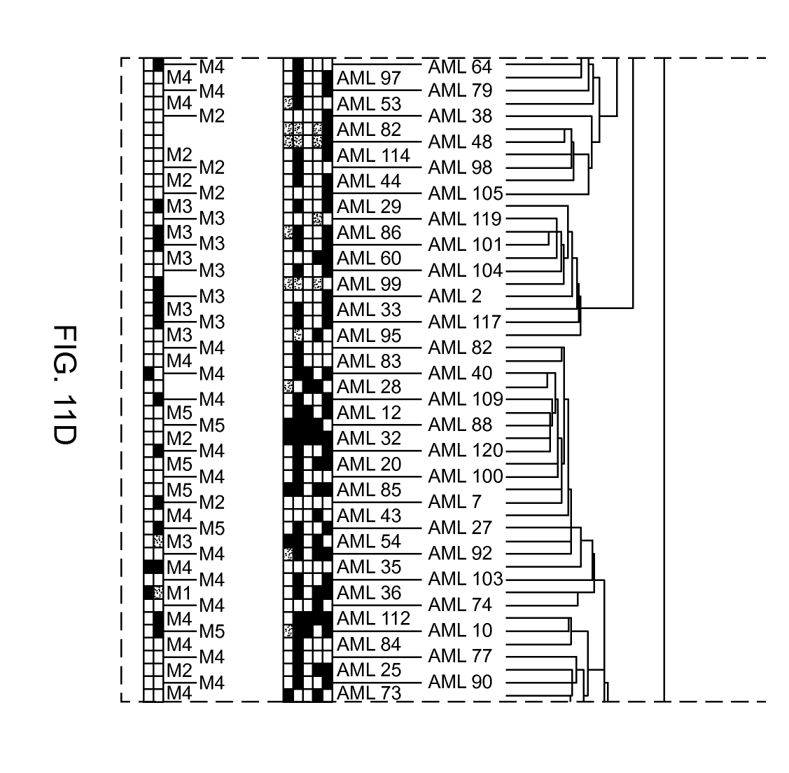

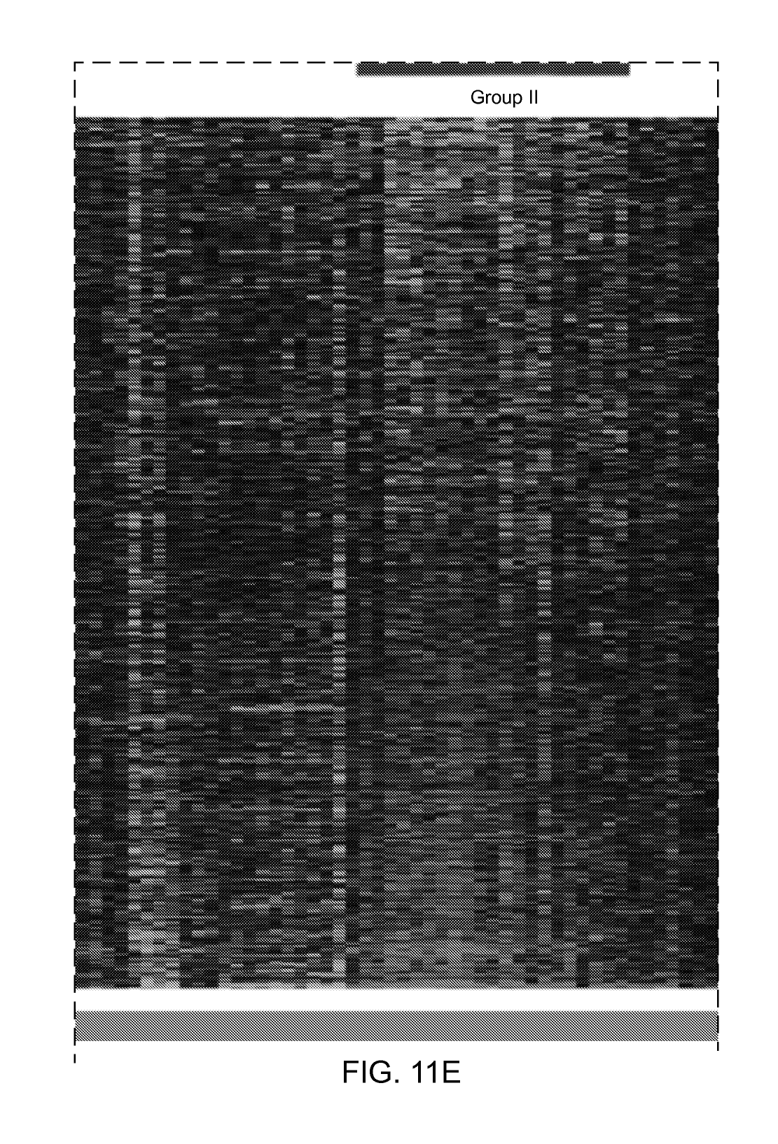

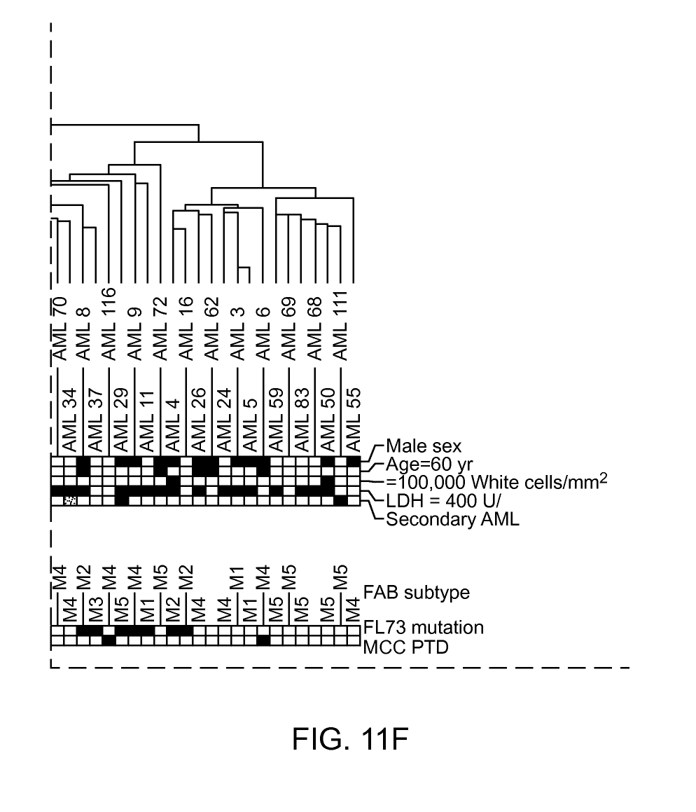

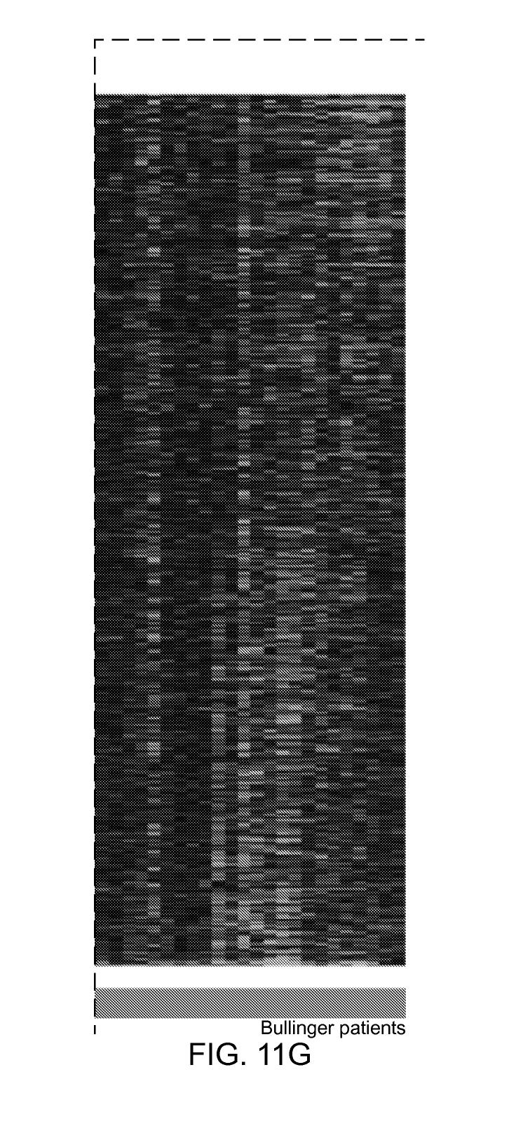

Since the data in Metzeler et al. represents only the samples from patients with normal karyotype, two more datasets covering a variety of known AML types were examined (Bullinger et al., supra, Kharas, 2010). The prognostic significance of the CD321 levels was found to be noticeably better in patients with normal karyotype (FIG. 7 D-I). To better associate the AML types defined by signaling in this study with AML classes described previously, gene expression in unsorted Type 1 and Type 2 AML samples (estimated by weighted sum of the microarray data from GRC and NGRC) was compared with two previously published AML cohorts (Bullinger et al., supra). Genes differentially expressed between the Type 1 and Type 2 samples were found to be differentially expressed between the Group 1 and Group 2 patients that were defined by the authors within a normal karyotype subset of their total data data (Bullinger et al., supra) (FIG. 11). With the exception of one patient with trisomy on chromosome 8 (NL027), all of the Type 1 and Type 2 patients analyzed in this study represent AML with normal karyotype. An independent observation of subtypes in AML with normal karyotype done in this study a) points to robustness of this sub-classification, b) reveals unique mechanisms underlying the immature gene expression signature observed in Type 2 and Group 1 patients, and c) establishes the link between the signaling response and classes as defined by gene expression in unsorted blasts.

### Discussion

Due to the inability to sense the extracellular cues or respond to them normally, cancer cells are generally locked in a “primitive” state that is prone to sustained activation of anti-apoptotic and proliferation pathways. The ability to differentiate is thought to be blocked by mutations in transcriptional regulators implicated in maturation programs. Yet despite being thought as generally “primitive”, with respect to cell maturity, many cancers and leukemia in particular manifest a variety of phenotypes that differ by the epistatic position of genetic lesion underpinning the maturation block (Largaespada, D. A. (2000). Genetic heterogeneity in acute myeloid leukemia: maximizing information flow from MuLV mutagenesis studies. Leukemia 14, 1174-1184; Heuser, M., et al. (2009). Modeling the functional heterogeneity of leukemia stem cells: role of STAT5 in leukemia stem cell self-renewal. Blood 114, 3983-3993). The activation of pro-survival pathways and of proliferation is linked to mutations that produce hyper-phosphorylated and constitutively activated intermediates of cytokine signaling pathways. Signaling patterns differ significantly between normal and cancer cells. More than 50% of AML patients manifest elevated basal levels of phosphorylated Stat5 that has been linked to activating mutations in Flt3, KIT, or JAK2 genes (Birkenkamp, K. U., et al. (2001). Regulation of constitutive STAT5 phosphorylation in acute myeloid leukemia blasts. Leukemia: official journal of the Leukemia Society of America, Leukemia Research Fund, UK 15, 1923-1931). Curiously though, there is a lack of direct correlation between the integrity of the Flt3 locus and the level of phosphorylated Stat5 in AML blasts (Pallis, M., et al. (2003). Flow cytometric measurement of phosphorylated STAT5 in AML: lack of specific association with FLT3 internal tandem duplications. Leukemia Research 27, 803-805; Schepers, H., et al. (2007). STAT5 is required for long-term maintenance of normal and leukemic human stem/progenitor cells. Blood 110, 2880-2888). We and others have recently reported that rather than basal levels, it is the abundance of cells responding to a diagnostic panel of cytokines that correlates with the Flt3 mutation status, response to chemotherapy and accordingly with survival prognosis (Irish et al., supra; Kornblau, S. M. et al., supra; Rosen et al., supra). It has been previously reported that the abundance of circulating of stem cells and progenitors as well as the detection of LSC gene expression signature is a significant factor in AML patient survival stratification (Berer et al., supra; Gentles et al., supra; Sutherland et al., supra). We sought to understand how the cells differentially responding to G-CSF relate to the cellular hierarchy of the AML proposed within the stem cell theory. For that matter, the gene expression difference between the AML cells responding and not responding to G-CSF in individual patient was evaluated. Techniques were established for isolation of intact RNA from permeabilized stained and sorted cells. Post-fixation following the initial fix and treatment with permeabilizing agents was important for successful isolation of RNA. The approach was validated on cells from normal donors and then used to study the gene expression in peripheral blasts of AML patients.

AML patients were classified into two groups according to gene expression data. In Type 1 patients, the cells that responded to G-CSF were primitive, whereas cells that did not respond to G-CSF were relatively mature. A set of surface markers including CD321, CD36, and CD11a enabled enrichment of the signaling subsets by in vivo sorting. The role of mature leukemic cells in amplification of leukemic stem cells has been previously documented (Dührsen et al., 1995). In Type 1 patients, the relatively mature cells that did not respond to G-CSF secrete cytokines that are able to induce Stat5 phosphorylation in subsets that do respond to G-CSF (FIG. 6). GM-CSF was detected in serum of most patients analyzed (data not shown). Interestingly, CD321 can serve as a ligand for CD11a/LFA-1 alpha polypeptide, which plays a central role in leukocyte intercellular adhesion and transvasation (Ostermann, G., et al. (2002). JAM-1 is a ligand of the β2 integrin LFA-1 involved in transendothelial migration of leukocytes. Nature Immunology 3, 151-158). These observations underscore cytokine feedback loops as a major factor contributing to sustainability of cancer lineage and indicate that the signaling subsets examined in this study may be the major purveyors of these interactions.

In PBMCs of Type 1 patients, the methylcellulose clonogenic activity co-enriched with the cells that responded to G-CSF within the CD321^{high} compartment. The analysis of publically available data from a large group of AML patients (Metzeler et al., supra) revealed that CD321 was inversely correlated with prognosis: cells from patients with high rates of survival expressed little CD321. This effect was even more prominent when examined in AML patients positive for the NPM1 mutation. Generally NPM1 positive AML patients comprise a group with a favorable prognosis (Thiede, C., et al. (2006). Prevalence and prognostic impact of NPM1 mutations in 1485 adult patients with acute myeloid leukemia (AML). Blood 107, 4011-4020), however our data reveals that CD321 levels can be used for further diversification of diagnosis in these patients. In agreement with previous reports suggesting a correlation of phosphor-Stat3/5-positive population with negative prognosis (Irish et al., supra), our observations on co-enrichment of immature phospho-Stat5-positive cells within the clonogenic CD321^{high} compartment and its capacity to predict AML outcome indicate that in Type 1 patients, signaling response can be used as surrogate marker of clonogenicity. In view of recent publications suggesting that IL-3 receptor chain (CD123) is a specific marker of AML LSC (Jordan et al., supra) and successful use of CD123 monoclonal antibody for targeting the LSC (Jin, L., et al. (2009). Monoclonal antibody-mediated targeting of CD123, IL-3 receptor alpha chain, eliminates human acute myeloid leukemic stem cells. Cell Stem Cell 5, 31-42), the response to IL-3 observed in the population of CD321^{high} cells votes in support of this hypothesis. CD321 is thus a useful as a diagnostic marker and a therapeutic tool for eradication of clonogenic cells.

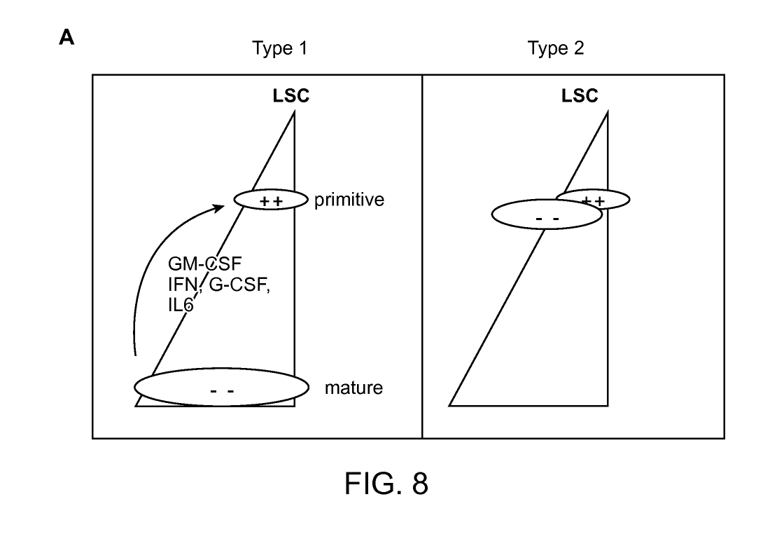

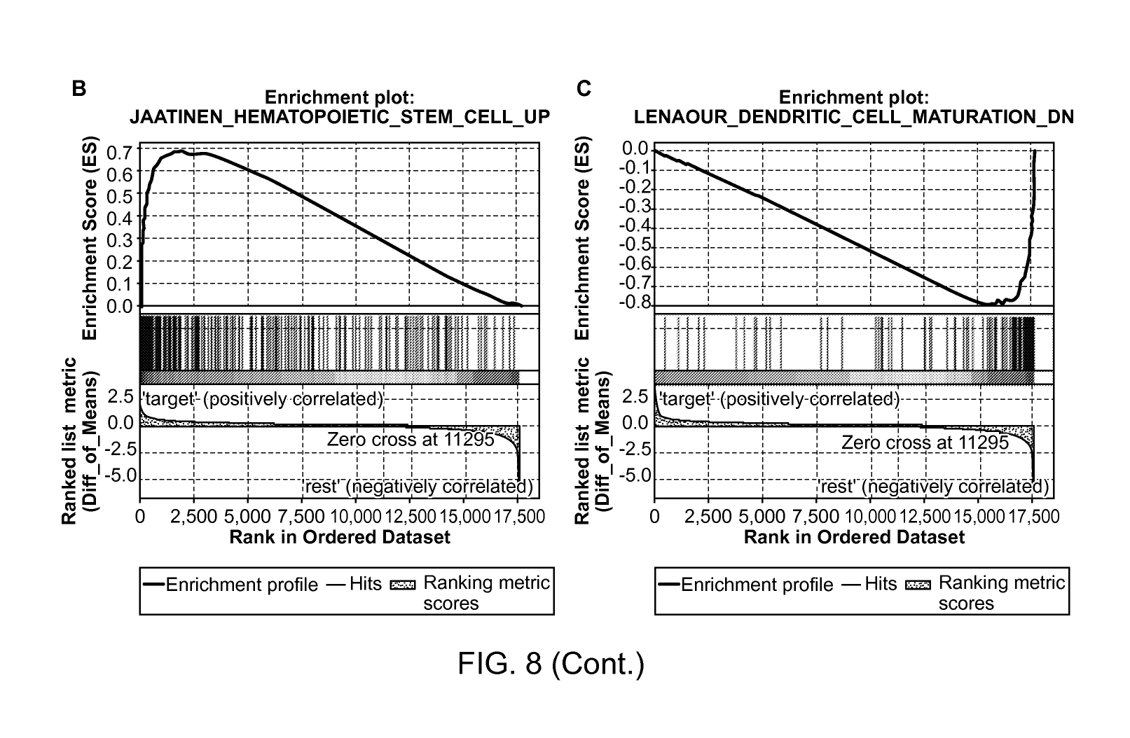

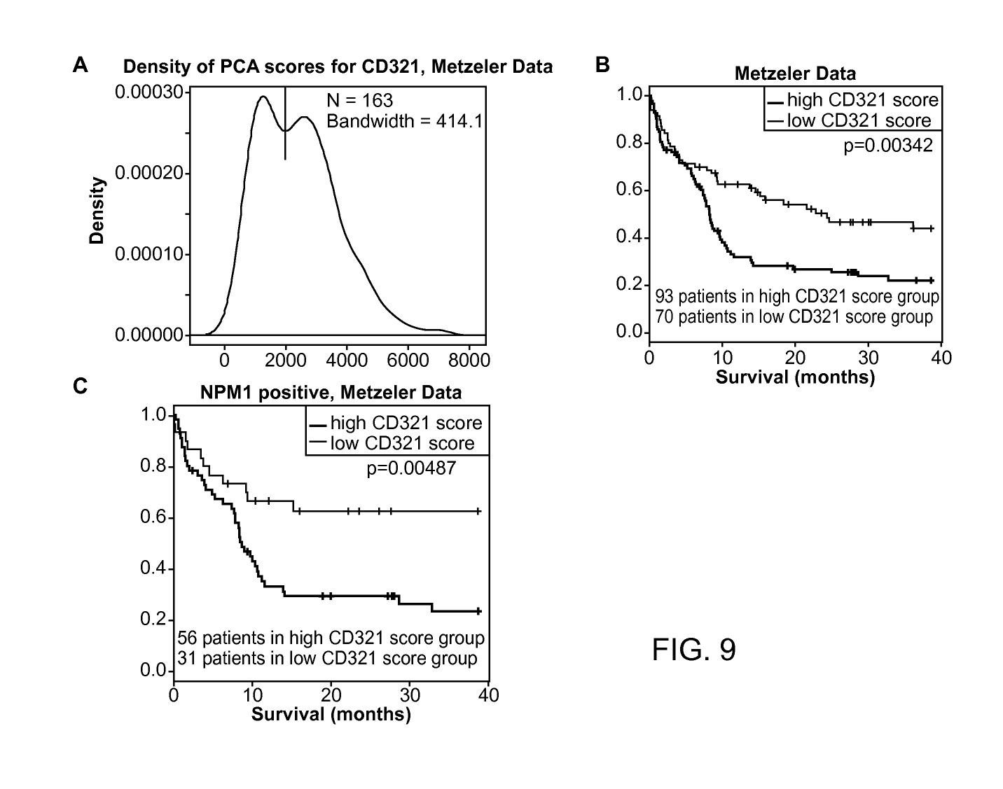

A response threshold of 13.5% effectively separated Type 1 from Type 2 patients; in Type 2 patient samples, more than 13.5% of cells respond to G-CSF with Stat phosphorylation. Expression profiling suggests that Type 2 patients have a type of AML that is significantly different from that afflicting the Type 1 patients. Gene set enrichment analysis of samples from Type 1 patients showed enrichment of genes specific to mature dendritic cells in NGRC and of stem cell specific genes in GRCs (FIG. 8 and Table 3 below). In contrast, there was no clear distinction between the NGRCs and GRCs of Type 2 patients: both signaling subsets were primitive and uniformly CD321^{high}CD36^{low}. Recent studies have underscored the fact that multiple cancer clones co-exist within the blast population of individual cancer patients both before and after relapse (Anderson et al., supra; Ding, L., et al. (2012). Clonal evolution in relapsed acute myeloid leukaemia revealed by whole-genome sequencing. Nature 481, 506-510; Notta, F., et al. (2011). Evolution of human BCR-ABL1 lymphoblastic leukaemia-initiating cells. Nature 469, 362-367). It is possible that cells manifesting different signaling responses belong to different co-existing clones within a patient; however, Copy Number Variation analysis on Affy SNP6.0 arrays performed on the subsets from one of the patients failed to detect any genetic differences between the subsets despite clear manifestation of additional rearrangements detected by same CGH procedure in cells before and after relapse in another patient (data not shown). The dynamic nature of Stat5 phosphorylation in response to G-CSF may be the explanation for the existence of responding and non-responding subsets in Type 2 patients. If this were the case, the proportion of cells responding to G-CSF in Type 2 patients may correlate with the half-life of phosphorylated Stat5. Future experiments should examine whether the half-life of phospho-Stat5 in Type 2 patients correlates with survival. Cross-dataset comparisons as well as comparative evaluation of CD321 prognostic performance indicate that Type 1 and Type 2 AMLs defined in this study are highly similar to prognostically distinct Group 1 and Group 2 of Normal Karyotype AML observed previously (Bullinger et al., supra) (based solely on unsorted gene expression data). While in that study it has not been explicitly stated that Group 1 and Group 2 represent AMLs with comparatively immature and differentiated blasts, several recent reports link the presence of HSC and LSC gene expression signatures with disease outcome (Eppert et al., supra; Gentles, supra). It is though yet unclear whether the AMLs with Normal Karyotype represent a homogeneous group where variable HSC abundance could be observed or whether unique dissimilar disease subtypes co-exist inside this class of patients. Our study supports the second model. In particular, a sharp difference between the NGRC of Type 1 and Type 2 AMLs suggest that these patients represent mechanistically distinct disease subtypes.

Table 3 (A) Top 10 genes expressed higher in cells responding to G-CSF in Type 1 patients. (B) Top 10 genes expressed higher in cells that do not respond to G-CSF in Type1 patients

TABLE 3A

| | | | Reported in |
|---|---|---|---|
| Gene | Protein function | Biological function | leukemia |
| MSI2 | RNA binding protein | Post-transcription | CML |
| | | regulation | |
| JDP2 | Transcription factor | AP-1 repressor | Tumoregensis |
| | | | or tumor |
| | | | repressor |
| HIC2 | Transcription factor | Unclear | Leukemia drug |
| | | | response |
| HOXB3 | Transcription factor | Differentiation | Leukemo- |
| | | | genesis |
| GATA2 | Transcription factor | Regulates WT-1 | AML, CML |
| | | | and MDS |
| HHEX | Transcription factor | Differentiation of | AML and CML |
| | | hematopoietic system | |
| MYB | Transcription factor | Differentiation and | AML |
| | | proliferation of | |
| | | hematopoietic | |
| | | progenitors | |
| IKZF3 | Transcription factor | Differentiation and | B-ALL |
| | | proliferation | |
| RHOH | Small GTPase | Leukocyte migration | B-CML, AML |
| | | | and hairy |
| | | | cell leukemia |
| TFDP2 | Transcription factor | Proliferation | Pediatric ALL |

TABLE 3B

| | | | Reported |
|---|---|---|---|
| Gene | Protein function | Biological function | in cancer |
| MAFB | Transcription | Lineage specific | Oncogenic |
| | factor | heamtopoiesis | or tumor |
| CCR1 | TMP | Chemokine response | suppressor |
| NOD2 | Intracellular | Immune response | |
| | receptor | | |
| CHST15 | Enzymatic | | |
| MPEG1 | unclear | | |
| FCN1 | TMP | Elastin binding of | |
| | | leukocytes | |
| LILRB2 | TMP | Inflammation and | |
| | | cytotoxicity | |
| LGALS2 | TMP | Differentiation and | |
| | | proliferation | |
| SLAMF7 | TMP | Maybe involved in NK | |
| | | cell activation | |
| CD86 | TMP | T cell activation | |

Experimentally obtained gene expression profiles were used to verify the ability of computational methods such as of linear modeling (Shen-Orr et al., 2010) to elucidate the subset-specific gene expression profiles. When applied to an unbiased (with respect to presence of Type1 and Type 2 patients) cohort, the deconvolution properly captured the average gene expression in two types of G-CSF induced signaling subsets.

The gene expression in signaling subsets induced by selected cytokines used in the 2004 study was predicted using the deconvolution algorithm (Shen-Orr et al., 2010). The analysis was performed on expression data from unsorted cells of selected AML patients (positive for the Flt3 tandem duplication) from the 2004 cohort (Irish et al., supra). Computational predictions indicate that cells that responded to GM-CSF, IL-3, or IFNγ with Stat5 and Stat3 phosphorylation occupy intermediate positions on the differentiation diagram, close to non-responding cells (although they are less mature). Cells that respond to IFNγ with Stat1 activation, like cells that respond to G-CSF with Stat5 and Stat3 activation, belong to the type proximal to leukemic stem cells, whereas cells that do not respond to IFNγ and G-CSF belong to the mature subset of the leukemic lineage. This data suggests that cytokine response profiling can be used to probe the architecture of the cancer clone.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

That which is claimed is:

1. A method for providing a diagnosis or a prognosis for an individual with leukemia, the method comprising,
measuring JAM-A in a hematologic sample from an individual, and
providing a diagnosis or prognosis based on said measuring.

2. The method according to claim 1, wherein the measuring comprises: contacting the sample with a JAM-A probe, and measuring the amount of JAM-A in the sample or the number of JAM-A+ cells in the sample.

3. The method according to claim 2, wherein the probe is an antibody.

4. The method according to claim 2, wherein the probe is a nucleic acid.

5. The method according to claim 2, wherein the method further comprises comparing the measurement to the measurement from a hematologic sample from a control.

6. The method according to claim 5, wherein the control is a sample from a healthy individual.

7. The method according to claim 5, wherein the control is a sample from an individual with leukemia.

8. The method according to claim 1, wherein the leukemia is AML.

9. The method according to claim 1, wherein the hematologic sample is a peripheral blood mononuclear cell (PBMC) sample.

10. The method according to claim 1, further comprising genotyping the patient for a NPM1 AML mutation.

11. A method for classifying a leukemia as a Type 1 or Type 2 AML, the method comprising:
measuring the number of JAM-A+ cells in a hematologic sample from an individual with a leukemia, and
classifying the leukemia as a Type 1 AML or a Type 2 AML based on the number of JAM-A+ cells.

12. The method according to claim 11, wherein the method further comprises comparing the number of JAM-A+ cells to the number of JAM-A+ cells in a control sample.

13. The method according to claim 11, wherein the hematologic sample is a peripheral blood mononuclear cell (PBMC) sample.

14. The method according to claim 11, wherein the method further comprises providing a prognosis based upon the classification, wherein a classification of Type1 AML indicates better overall survival for the AML patient and a classification of Type 2 AML indicates worse overall survival for the AML patient.

15. A method for classifying a leukemia as a Type 1 or Type 2 AML, the method comprising:
stimulating with G-CSF a population of blast cells from a hematologic sample from an individual with leukemia;
measuring the number of G-CSF-responsive cells (GRCs) and/or the number of G-CSF non-responsive cells (NGRCs) in the stimulated sample; and
classifying the leukemia as a Type 1 AML or a Type 2 AML based on the number of GRCs and/or NGRCs in the stimulated sample.

16. The method according to claim 15, wherein the method further comprises providing a prognosis based upon the classification, wherein a classification of Type1 AML indicates better overall survival for the AML patient and a classification of Type 2 AML indicates worse overall survival for the AML patient.

17. A method for depleting G-CSF-responsive cells (GRCs) in an individual with leukemia, the method comprising:
contacting the G-CSF-responsive cells (GRCs) with a JAM-A-binding agent in an amount effective to deplete the GRCs.

18. The method according to claim 17, wherein the method occurs ex vivo, and the method further comprises returning the non-GRCs to the individual.

19. The method according to claim 18, wherein the JAM-A binding agent is conjugated to a therapeutic moiety that promotes cell death in GRCs.

20. The method according to claim 18, wherein the JAM-A binding agent is an affinity reagent that is used to remove the contacted GRCs.

21. The method according to claim 17, wherein the contacting occurs in vivo, wherein the JAM-A binding agent is conjugated to a therapeutic moiety that promotes cell death in GRCs.

22. The method according to claim 21, wherein the therapeutic moiety is a cytotoxin or a polypeptide that targets the cell for ADCC or CDC-dependent death.

23. The method according to claim 21, wherein the therapeutic moiety is a small molecule or polypeptide that alters the activity of the cell.

24. The method according to claim 17, wherein the method treats the individual for leukemia.

25. The method according to claim 24, wherein the leukemia is AML.

26. A method for modulating the activity of G-CSF responsive cells (GRCs) in an individual, the method comprising:
administering to the individual an effective amount of an agent that modulates the activity of the JAM-A signaling pathway.

27. The method according to claim 26, wherein the method treats an individual for leukemia.

28. The method according to claim 26, wherein the leukemia is AML.

29. A method for screening a candidate agent for the ability to treat leukemia, the method comprising:
comparing the viability, proliferation rate, or metastatic potential of JAM-A+ cells contacted with agent with the viability, proliferation rate, or metastatic potential of JAM-A+ cells not contacted with agent,
wherein a decrease in viability, proliferation rate, or metastatic potential in the JAM-A+ cells contacted with agent indicates that the agent is able to treat leukemia.

30. The method according to claim 29, wherein the cells are contacted with a JAM-A modulator.

31. The method according to claim 29, wherein the cells are contacted with one or more cytokines that activate JAM-A+ cells.

32. The method according to claim 29, wherein the leukemia is AML.
